(19) United States
(12) Patent Application Publication
(10) Pub. No.: US 2002/0169562 A1
(43) Pub. Date: Nov. 14, 2002

(54) Defining biological states and related genes, proteins and patterns

(76) Inventors: Gregory Stephanopoulos, Chester, MA (US); Jatin Misra, Cambridge, MA (US); Daehee Hwang, Cambridge, MA (US); William A. Schmitt JR., Boston, MA (US); Ilias Alevizos, Watertown, MA (US); Saliya Sudharshana Silva, Kandy (LK); Ryan T. Gill, Boulde, CO (US)

Correspondence Address: FOLEY HOAG LLP, PATENT GROUP, 155 SEAPORT BOULEVARD, BOSTON, MA, 02110, US

(21) Appl. No.: 10/060,048

(22) Filed: Jan. 29, 2002

Related U.S. Application Data
(60) Provisional application No. 60/285,186, filed on Apr. 20, 2001.
(60) Provisional application No. 60/264,779, filed on Jan. 29, 2001.

(51) Int. Cl. G01N 33/48; C12Q 1/68; C07K 1/00; C07H 21/00

(52) U.S. Cl. 702/19; 435/6; 536/23.1; 530/350

(57) ABSTRACT

Disclosed are a variety of methods and computer systems for use in the analysis of gene and protein expression data. Also disclosed are methods for the definition of the cellular state of cells and tissues from multidimensional physiological data such as those obtained from gene expression measurements with DNA microarrays. A variety of classification methods can be applied to expression data to achieve this goal. Demonstrated is the application of several statistical tools including Wilks' lambda ratio of within-group to total variance, Fisher Discriminant Analysis, and the misclassification error rate to the identification of discriminating genes and the overall classification of expression data. Examples from several different cases demonstrate the ability of the method to produce well-separated groups in the projection space representing distinct physiological states. The method can be augmented and is useful in disease diagnosis, drug screening and bioprocessing applications.

### CROSS-REFERENCE TO RELATED APPLICATIONS

This application claims the benefit of priority under 35 U.S.C. section 119(e) to Provisional Patent Applications 60/285,186 filed Apr. 20, 2001, and 60/264,779 filed Jan. 29, 2001. These applications are hereby incorporated by reference in their entirety.

### STATEMENT REGARDING FEDERALLY SPONSORED RESEARCH

The subject invention was made in part with support from the Engineering Research Program of the Office of Basic Energy Science at the Department of Energy, Grant No. DE-FG02-94ER-14487 and DE-FG02-99ER-15015. Additional support was provided by NlH grant number 1-RO1-DK58533-01. Accordingly, the U.S. Government has certain rights in this invention.

### BACKGROUND

1. The Relationship Between Gene and Protein Expression and Cellular Processes

Cell function is the integrated outcome of numerous cellular processes and can be described by different combinations of parameters reflecting to varying extents such intracellular processes. In the simplest case, growth is used as an all-encompassing physiological descriptor. Growth (and growth rate) are usually supplemented by an array of extracellular variables in describing cell function and physiology, such as respiration rate, rate of glucose consumption, lactate accumulation, etc. This vector of physiological variables can be further augmented by derivative quantities such as the rates of glycolysis, TCA cycle activity, pentose phosphate pathway flux, etc. by invoking overall intracellular metabolite balances. These balances can then be solved for the unknown intracellular fluxes as functions of the extracellular metabolite accumulation rates. Vallino, J. J. and G. Stephanopoulos, “Metabolic Flux Distributions In Corynebacterium-Glutamicum During Growth and Lysine Overproduction.” Biotechnology and Bioengineering, 41, 633-646, (1993); Vangulik, W. M. and J. I Heijnen, “A Metabolic Network Stoichiometry Analysis Of Microbial-Growth and Product Formation.” Biotechnology and Bioengineering, 48, 681-698, (1995); Stephanopoulos, G., Metabolic Engineering, 1, 1-11 (1999).

Cell function, as described by the above variables, is the expression of a particular cellular state that can be quantified by a variety of methods probing the transcriptional, proteomic and metabolic state of a cell. Since all cellular processes originate at the transcription level, it can be argued that transcriptional profiling provides a broad descriptor of the cellular physiological state. Consequently, gene transcription measurements by various types of microarrays contain information that should be useful, in principle, in defining the physiological state of a cell. Schena, M., D. Shalon, R. W. Davis and P. 0. Brown, “Quantitative Monitoring Of Gene-Expression Patterns With a Complementary-Dna Microarray.” Science, 270, 467-470, (1995); Lockhart, D. J., H. L. Dong, M. C. Byrne, M. T. Follettle, M. V. Gallo, M. S. Chee, M. Mittmann, C. W. Wang, M. Kobayashi, H. Horton and E. L. Brown, “Expression monitoring by hybridization'to high-density oligonucleotide arrays.” Nature Biotechnology, 14, 1675-1680,(1996). However, although no one doubts the value of information residing in microarray expression data (collectively, referred to as the expression phenotype), it has not been clear how these data can be used in a definition of the physiological state of a cell.

The sheer magnitude of the expression phenotype has forced many investigators to focus on a small number of gene or protein expression measurements in attempting to define the physiological state. Concentrating on only a few genes implies that, despite its apparent complexity, cellular function is still determined by a small number of genes, a corollary that is not supported by the accumulating evidence of microarray data. Golub, T. R., D. K. Slonim, P. Tamayo, C. Huard, M. Gaasenbeek, J. P. Meslrov, H. Coller, M. L. Loh, J. R. Downing, M. A. Caligiurl, C. D. Bloomfield and E. S. Lander, “Molecular classification of cancer: Class discovery and class prediction by gene expression monitoring.” Science, 286, 531-537, (1999). Consideration of combinations of gene or protein expressions, on the other hand, involves the entire expression phenotype in the definition of cell physiology and also allows the relative importance of the expression of each gene to be determined. Analytical methods permitting this type of analysis would have applications ranging from medicine to industrial biology to basic science, extending to essentially any situation where a change in gene or protein expression may be connected to a change in cellular state.

2. Hyperproliferative Disorders

Carcinomas and other hyperproliferative disorders involving transformation of epithelial cells are amongst the most prevalent forms of cancer. Oral cavity cancer, for instance, is the sixth most common cancer in the United States. It is newly diagnosed in about 31,000 Americans each year and 350,000 people worldwide. One patient dies from oral cancer every hour in the U.S. alone.

Cancers of the mouth present in various forms. Any persistent white patch must be regarded as being suspicious. Additionally, velvety red patches- particularly those with white speckles- should be areas of concern. Finally, any non-healing ulcer (erosion) merits evaluation. More often than not, these areas are painless. The tongue is the most common site of oral cancer. Typically, the side of the tongue (farthest back in the mouth) is involved. The floor of the mouth (that area beneath the tongue) is next in order of frequency followed by the insides of the cheeks with involvement of other areas showing a lesser incidence.

Oral squamous cell carcinoma, for example, has been linked to excessive cigarette smoking and alcohol abuse, both individually and in combination. Other factors associated with oral cancer include poor dental hygiene and malfitting dentures or broken teeth that cause chronic mucosal irritation. Occupational hazards include chronic dust exposure among woodworkers, which has been associated with cancer of the nasopharynx, and exposure to nickel compounds, which increases the risk of paranasal sinus cancers.

About 90% of oral cancers are detected in only a few high-risk sites; the floor of the mouth, the ventrolateral aspect of the tongue, and the soft palate complex. Buccal and labial vestibular carcinoma should be considered in people who use smokeless tobacco.

Early, asymptomatic oral cancer appears most often as a red (erythroplastic) lesion. Squamous cell carcinoma, not diagnosed in its earliest stages appears later as a deep ulcer with smooth, indurated, rolled margins, fixed to deeper tissues. Biopsy is necessary to diagnose carcinoma.

Squamous cell carcinomas are often diagnosed early because such cancers lead to local symptoms such as pain, hoarseness, and difficulty in swallowing. In many cases, however, diagnosis is delayed because local symptoms or pain from nerve involvement does not occur until a large primary tumor develops. In such cases, regional nodal metastases may be the initial manifestation. Distant metastases rarely occur without locally advanced primary disease or nodal involvement.

Improved methods for classifying and diagnosing hyperproliferative disorders would be invaluable in the medical field. In addition, much current research is concerned with the identification of genes that are mechanistically related to the transformation of a non-hyperproliferative cell to a hyperproliferative cell.

3. Polyhydroxyalkanoic Acids

Polyhydroxyalkanoic acids (PHA) form a class of biopolymers, of which polyhydroxybutyric acid (PHB) is a member, that can be synthesized by many genera of bacteria and whose properties can vary over a large range. Doi, Y. 1990. Microbial Polyesters. VCH Publishers. Over 90 different members of the PHA class of biopolymers have been discovered each of which differs slightly in the number of carbons in the monomeric sub-unit or the structure of the pendant side chain. Steinbuchel, A., and B. Fuchtenbushc. 1998. Bacterial and other biological systems for polyester production. TIBTECH 16:419-426. PHAs are characterized by a polyester backbone and a diverse set of side-chain structures that provide considerable flexibility in PHA polymeric properties. Steinbuchel, A., and H. Valentin. 1995. Diversity of bacterial polyhydryoxyalkanoic acids. FEMS Microbiology Letters 128:219-228. Importantly, members of the PHA biopolymer family are biodegradable and are therefore of interest as an alternative to petrochemical based polymers. Biologically based processes for the production of PHAs have been established in the past 25 years with current bioproduction systems capable of PHA accumulation levels close to 80% of dry cell weight and productivities of almost 5 g/L-hr. Fidler, S., and D. Dennis. 1992. Polyhydroxyalkanoate production in recombinant Escherichia coli. FEMS Microbiology Reviews 9:231-235; Peoples, O., and A. Sinskey. 1989. Poly-beta-hydroxybutyrate biosynthesis in Alcaligenes eutrophus H16. Identification and characterization of the PHB polymerase gene (phbC). Journal of Biological Chemistry 264:15298-15393; Slater, S., T. Gallaher, and D. Dennis. 1992. Production of poly-(3-hydroxybutyrate-co-3-hydroxyvalerate) in a recombinant Escherichia coli strain. Applied Environmental Microbiology 58:1089-1094; Stal, L. 1992. Poly(hydroxyalkanoate) in cyanobacteria: an overview. FEMS Microbiology Reviews 103:169-180; Steinbuchel, A., and B. Fuchtenbushc. 1998. Bacterial and other biological systems for polyester production. TIBTECH 16:419-426; Lee, S., J. Choi, and H. Wong. 1999. Recent advances in polyhydroxyalkanoate production by bacterial fermentation: mini-review. International Journal of Biological Macromolecules 25:31-36; Liu, S., and A. Steinbuchel. 2000. A novel genetically engineered pathway for synthesis of poly(hydroxyalkanoic acids) in Escherichia coli. Appplied Environmental Microbiology 66:739-743; Park, S., W. Ahn, P. Green, and S. Lee. 2001. Biosynthesis of poly(3-hydroxybutyrate-co-3-hydroxyvalerate-co-3-hydroxyhexanoate) by metabolically engineered Escherichia coli strains. Biotechnology and Bioengineering 74:81-86. One of the main limitations to the commercialization of PHA bioprocesses, however, has been -the use of expensive carbon substrates. Choi, J., and S. Lee. 2000. Economic considerations in the production of poly(3-hydroxybutyrate-co-3-hydroxyvalerate) by bacterial fermentation. Applied Microbiology and Biotechnology 53:646-649; Lee, S., J. Choi, and H. Wong. 1999. Recent advances in polyhydroxyalkanoate production by bacterial fermentation: mini-review. International Journal of Biological Macromolecules 25:31-36.

Synechocystis is a photosynthetic cyanobacterium that is capable of utilizing carbon dioxide as a primary carbon source and that naturally accumulates PHB as a reserve for excess carbon and reducing power. Stal, L. 1992. Poly(hydroxyalkanoate) in cyanobacteria: an overview. FEMS Microbiology Reviews 103:169-180; Taroncher-Oldenburg, G., K. Nishihara, and G. Stephanopoulos. 2000. The Physiology of PHB accumulation in Synechocystis sp. PCC6803. Applied Environmental Microbiology:In Press. PHA accumulation is known to be positively affected by the presence of excess carbon and by a limitation in another essential nutrient such as nitrogen or phosphate. Lee, S., J. Choi, and H. Wong. 1999. Recent advances in polyhydroxyalkanoate production by bacterial fermentation: mini-review. International Journal of Biological Macromolecules 25:31-36; Steinbuchel, A., and B. Fuchtenbushc. 1998. Bacterial and other biological systems for polyester production. TIBTECH 16:419-426; Wu, G., Q. Wu, and Z. Shen. 2001. Accumulation of poly-β-hydroxybutyrate in cyanobacterium Synechocystis sp. PCC6803. Bioresource Technology 76:85-90. The ability of Synechocystis to grow on essentially free carbon and energy sources (CO₂ and sunlight), generated interest in exploring its possible use as a system for the inexpensive production of biopolymers. Miyake, M., K. Takase, M. Narato, E. Khatipov, J. Schnackenberg, M. Shirai, R. Kurane, and Y. Asada. 2000. Polyhydroxybutyrate production from carbon dioxide by cyanobacteria. Applied Biochemistry and Biotechnology Spring:991-1002.

### BRIEF SUMMARY

In one aspect, the invention relates to methods for use in the analysis of gene or protein expression information, where such methods involve the determination of a within group measure of variability and a total (and/or between group) measure of variability. In other aspects, the invention relates to the identification of genes or proteins, sets of genes or proteins, and patterns of gene and protein expression that are related to cellular states and/or changes in cellular states.

In one embodiment, a method for use in analyzing gene or protein expression data comprises accessing gene or protein expression data that includes expression levels of G genes or proteins in S samples, where the S samples may be classified into C classes representing cellular states (G, S and C may be essentially any number); determining a measure of the variability of expression levels of each gene or protein in the data as a whole; and determining a measure of the variability of expression levels of each gene or protein within each class of sample. In some embodiments, the invention further comprises determining a between group measure of variability by determining the difference between the total and within group measures of variability.

In certain embodiments, the method includes comparing one or more of the determined measures of variability, by, for example, calculating a ratio between the measures of variability, calculating a Wilks' lambda score, scaling one measure of variability by another, etc.

In general, the methods for analysis are amenable to any type of data set. For example, methods described herein may be applied to data relating to the expression of a single gene or protein. In other embodiments, the methods may be applied to date relating to the expression levels of two, three, five, ten, twenty, fifty, one hundred, one thousand, ten thousand or more genes or proteins. In addition, gene or protein expression levels may be measured in one or more samples, and preferably more than one sample that can be classified into two or more classes that represent cellular states (in general, the terms “biological state”, “cellular state” and “physiological state” are used interchangeably herein).

In certain embodiments, the invention relates to matrix methods of analysis using the statistical methods described above. In certain embodiments, the data is organized into a data matrix Xₖ for each class k, and wherein each data matrix is organized such that X(i,j) is the expression of gene j in sample i. In further embodiments, between group measures of variability may be represented by a matrix B, within group measures of variability may be represented by the matrix W, and total variability may be represented by T. In some embodiments, measures of variability are compared by forming the matrices W⁻¹B and or W⁻¹T.

In some embodiments, the invention relates to methods for maximizing the separation between the classes by generating a reduced dimensional space into which gene or protein expression data may be projected. For example, the separation between the classes in a reduced dimensional space may include generating an eigenvector matrix L of the matrix W⁻¹B and an eigenvalue matrix Λ of the matrix W⁻¹B, optionally wherein a column of L defines a discriminant function of the reduced dimensional space, and wherein each entry in the column indicates the contribution of each gene to the discriminant function. In another example, maximizing the separation between the classes in a reduced dimensional space may include generating a singular value decomposition of the matrix W⁻¹B, optionally according to the formula W⁻¹B=UΛL^{T} where U is a left singular vector, L is a matrix of discriminant functions, and Λ is a matrix of singular values representing the discriminant loadings in the corresponding functions.

In certain embodiments, the methods of the invention comprise calculating a discriminator vector for each sample, wherein the discriminator vector represents a position of the sample in the reduced dimensional space. Optionally, samples may be compared by comparing their vectors or positions in the reduced dimensional space, and samples of similar cellular state tend to have similar discriminator vectors. In certain embodiments, the method comprises operating the formula:

$${y}_{j} = i {L}_{j} = {\sum }_{z = 1}^{g} {i}_{z} {L}_{ij}$$

wherein yⱼ is the discriminator score of the sample i (a sample of g genes) for each column j of matrix L, and wherein the discriminator vector is a combination of each yⱼ into a vector having a dimensionality that is equal to the number of dimensions in the reduced dimensional space.

In some embodiments, the methods comprise generating discriminant loadings based on the comparison of measures of variability. In certain exemplary embodiments, the discriminant loadings may be viewed as the coefficients by which a data set for a sample is to be multiplied in order to generate a discriminator vector for that sample. Optionally, the contribution of the expression levels of a gene or protein to the discriminant loadings may be determined, and a gene or protein that contributes significantly to a discriminant loading is a gene or protein that is related to a cellular state or a change in cellular state of one or more sample. Optionally, genes or proteins may be ranked on the basis of contributions to the discriminant loading, and such contributions may be compared by comparing the F score for each gene or protein.

In certain aspects, the invention relates to methods for identifying genes or proteins or groups of genes or proteins, the expression levels of which are related to a cellular state or a change in cellular state. In one embodiment, a method comprises accessing gene or protein expression data comprising expression levels of G genes or proteins in S samples, where the S samples may be classified into C classes representing cellular states; determining a measure of the variability of expression levels of each gene or protein in the data as a whole (total) or between classes (between group); determining a measure of the variability of expression levels of each gene or protein within each class of sample (within group); and identifying a gene or protein which is related to a cellular state or a change in cellular state by identifying a gene or protein for which the within group measure of variability is less than the total or between group measure of variability with an 80% or higher degree of confidence, optionally a 90% or higher degree of confidence, optionally a 95% or higher degree of confidence and optionally a 99% or higher degree of confidence. Genes identified in this manner may, for example, be causally related to a change in one of the cellular states, or it may simply be a phenotype associated with differences between biological states.

In certain embodiments, genes or proteins (and optionally the expression levels thereof) identified according to the methods described herein may be used in a variety of applications including but not limited to: classification of samples of an unknown state (eg. diagnosis of disease states, monitoring of cell cultures etc.), screening assays to identify therapeutics that modify expression of a relevant gene or protein, engineering (genetically or otherwise) to alter the expression of an identified gene or protein, etc. As noted above, the method may include identifying a plurality of genes or proteins wherein the plurality of genes or proteins is a gene or protein group related to a cellular state or a change in a cellular state, and such sets of genes or proteins may be used similarly. In addition, the classification of genes or proteins into sets may illuminate previously unappreciated relationships between genes or proteins and may help define the function of genes and proteins of unknown function.

In certain embodiments, a set of contributing genes may be refined by generating a subgroup by omitting the gene or protein of interest from the group and testing the power of the subgroup to discriminate between two or more classes of sample. This calculation may be achieved, for example, by performing a leave one out cross-validation.

In certain aspects, the invention relates to the identification of a gene or protein expression pattern that is useful for discriminating between samples of two or more cellular states. In some embodiments, such a methods may comprise accessing gene or protein expression data comprising expression levels of G genes or proteins in S samples, where the S samples may be classified into C classes representing cellular states; determining a measure of the variability of expression levels of each gene or protein in the data as a whole (total) and/or determining a between group measure of variability; determining a measure of the variability of expression levels of each gene or protein within each class of sample (within group); generating for each gene or protein a comparison of the within group measure of variability to the total or between group measures of variability; and selecting from among the genes or proteins of a set of genes or proteins and corresponding expression levels that discriminate between two or more classes of sample with a misclassification rate less than 40%, optionally less than 30%, less than 20%, less than 15% or less than 10%. Such patterns of genes or proteins and corresponding expression levels may be useful for a variety of applications, including discriminating between samples of two or more cellular states.

In further aspects, the invention relates to methods of classifying the biological state of a sample by accessing gene or protein expression data from the sample and multiplying the data (either multiplication, vector multiplication or matrix multiplication, as appropriate) by discriminant loadings calculated from two or more control samples to generate a discriminant vector corresponding to the unclassified sample. The unclassified sample may then be compared to the two or more control samples, a the sample may be classified on the basis of mathematical similarity to one or more of the control samples.

In certain aspects, the invention relates to computer products for use in analyzing gene or protein expression data, where generally the product is disposed on a computer readable medium, and comprises instructions for causing a processor to perform any of the various analytical methods described above.

In further aspects, the invention relates to systems comprising a processor and instructions for causing a processor to perform any of the various analytical methods described above.

In yet further aspects, the invention relates to methods for use in modifying the production of a metabolite in a cell comprising accessing data comprising a representation of the expression levels of G genes or proteins in S samples, wherein the S samples may be classified into C classes representing biological states, and wherein at least two of the biological states differ in the level of the metabolite that is produced; and identifying a discriminating gene or protein, the expression levels of which are discriminatory in defining a biological state of higher metabolite production from a biological state of lower metabolite production. In general, identifying the discriminatory gene may be accomplished through a variability-based method as described above, or, optionally, through a different method such as a Pearson correlation, an entropy based method, etc. In further embodiments, the method may include identifying a discriminating pattern of gene or protein expression levels. In yet another embodiment the method may comprise selecting a cell having a desired level of metabolite production by identifying a cell having a pattern of expression levels that is mathematically similar to the discriminating pattern. In another embodiment, the method further comprises modulating the expression of the discriminating gene or protein in a cell, where such manipulation may be accomplished through a genetic change (eg. introduction of a transgene, mutation, etc.) or through a change in growth conditions (eg. nutrients, light, the presence of certain pharmaceuticals or other bioactive compounds etc.) or through other manipulations. The method of claim, further comprising evaluating the production of polyhydroxyalkanoate in the cell. Optionally, a bacterium, a cyanobacterium, a bacterium capable of photoautotrophic or chemoautotrophic growth and/or a bacterium selected from the group consisting of: Synechocystis spp., Synechococcus spp., Ralstonia eutropha, Alcaligenes latus, Azotobacter vinelandii and recombinant Escherichia coli. Preferably the cell is a bacterium of the strain Synechocystis sp. PCC6803. In certain embodiments at least one class represents a biological state selected from the group consisting of: a standard culture medium for the cell; a nitrogen-limited growth condition; a phosphorus limited growth condition; a light limited growth condition; and a growth condition where the culture medium is supplemented with a carbon source. Optionally at least one class represents a biological state selected from the group consisting of: a stationary phase culture; a lag phase culture; an exponentially growing culture; and a culture maintained at a steady state growth rate. Optionally at least one class represents a biological state selected from the group consisting of: standard BG11 medium; BG11 medium with reduced nitrate content; BG11 medium with reduced phosphate content; BG11 medium supplemented with acetate; BG11 medium with reduced nitrate content and supplemented with acetate; and BG11 medium with reduced phosphate content and supplemented with acetate.

In another aspect, the invention relates to a set of genes and a pattern of gene expression that is associated with differing levels of polyhydroxyalkanoate (PHA) production. Examples of such genes are provided in Table 1 below. In certain embodiments, the invention relates to altering the expression of one or more of these genes to improve PHA production, and in some embodiment, the altering involves the genetic makeup of the cell so as to cause the cell to have a modified expression of one of the genes. It is also understood that similar improvements in PHA production may be achieved through the manipulation of an orthologue of any of the preceding genes in the appropriate organism. A cell may be essentially any cell capable of producing PHAs, optionally a cyanobacterial cell and optionally a bacterial cell of the species Synechocystis sp., Synechococcus sp., Ralstonia eutropha, Alcaligenes latus, Azotobacter vinelandii, Anacystis nidulans or recombinant Escherichia coli. PHAs might include, but are not limited to any of the following: polyhydroxyproprionate, polyhydroxybutyrate, polyhydroxyvalerate, polyhydroxycaproate, polyhydroxyheptanoate, polyhydroxyoctanoate, polyhydroxynonanoate, polyhydroxydecanoate, polyhydroxyundecanoate, polyhydroxydodecanoate and a mixed polymer of one or more of the forgoing polymers, and, for example 3-hydroxyproprionate, 3-hydroxybutyrate, 4-hydroxybutyrate, 5-hydroxybutyrate, 3-hydroxyvalerate, 3-hydroxycaproate, 3-hydroxyheptanoate, 3-hydroxyoctanoate, 3-hydroxynonanoate, 3-hydroxydecanoate, 3-hydroxyundecanoate and 3-hydroxydodecanoate.

The invention further relates to bacteria comprising a recombinant nucleic acid construct comprising a coding sequence of a gene represented by an index number selected from the group consisting of: s110008, s110010, s110039, s110322, s110361, s110373, s110374, s110379, s110385, s110396, s110459, s110469, s110477, s110486, s110550, s110558, s110703, s110873, s111317, s111376, s111473, s111504, s111514, s111611, s111623, s111630, s111632, s111702, s111820 and s1r1822, or an orthologue of any of the preceding, and optionally two, three, four five or more of the above genes or orthologues. Optionally the bacteria is a cyanobacteria or alternatively the bacteria may be Synechocystis sp., Synechococcus sp., Ralstonia eutropha, Alcaligenes latus, Azotobacter vinelandii, Anacystis nidulans or Escherichia coli. In a further aspect, the invention provides methods for producing polyhydroxyalkanoate by culturing one of the foregoing cells. Such methods may further comprise obtaining PHA from the culture and various refinement steps. The PHA may be mixed with other plastics, pigments etc., and it may also be incorporated in consumer products for sale.

In yet another aspect, the invention relates to methods for determining whether a sample contains a hyperproliferative cell comprising: determining a level of gene expression of at least one gene in a sample, wherein the at least one gene is selected from the group consisting of Neuromedin U; Aldehyde dehydrogenase 9 (Human gamma-aminobutyraldehyde dehydrogenase E3 isozyme); Fibroblast growth factor 8; Human epidermal growth factor receptor (HER3); Translocase of outer mitochondrial membrane 34; KIAA0089; Monoamine oxidase B; Zinc finger protein 273; clone 1D2; Aldehyde dehydrogenase 10 (fatty aldehyde dehydrogenase); Carboxylesterase 2 (intestine, liver); Gro2 oncogene; Diazepam binding inhibitor; Cadherin 17; TAL1 (SCL) interrupting locus; Crystallin alpha B; 5T4 oncofetal trophoblast glycoprotein; Deoxyribonuclease I-like 3; Heat-shock protein 90-kDa; Smg GDS-associated protein; Cytochrome c oxidase subunit Vb (coxVb); Wilm Tumor-Related Protein; TYRO3 protein tyrosine kinase; FAT tumor suppressor; Creatine kinase, mitochondrial 1; Transcription factor 20; MHC class I polypeptide related sequence A; KIAA0018 gene product 1; Lectin galactoside-binding, soluble, 7 (galectin 7); Tenascin-R (restrictin, janusin); CD1A antigen, a polypeptide; Beta-Hexosaminidase, Alpha Polypeptide, Abnormal Splice Mutation; clone 1A7; KIAA0172 gene; Myxovirus (influenza) resistance 2, homolog of murine; Lysophospholipase like; Interleukin-8 receptor type B, splice variant IL8RB9; keratin 4; and Runt-related transcription factor, and wherein the level of gene expression of the at least one gene allows classification of an oral keratinocyte as hyperproliferative or non-hyperproliferative with a misclassification rate of 40% or lower; comparing the level of gene expression of said at least one gene to a first control level of gene expression of said at least one gene as measured in a hyperproliferative cell; and comparing the level of gene expression of the at least one gene to a second control level of gene expression of said at least one gene as measured in a non-hyperproliferative cell; wherein a sample contains a hyperproliferative cell if the level of gene expression of the at least one gene is more mathematically similar to the first control level of gene expression than to the second control level of gene expression. In certain embodiments, the method may comprise determining the expression levels of at least two genes, optionally at least three genes, five genes, ten genes, twenty genes, thirty genes, or at least forty genes. Furthermore, the misclassification rate may optionally be less than 30%, less than 20%, less than 15%, or less than 10%.

In a further aspect, the invention relates to methods for classifying leukemia using the list of genes provided in table 3. In one embodiment, the method comprises determining a level of gene expression of at least one gene in a sample, wherein said at least one gene is selected from the group consisting of U05259, M89957, M84371, D88270, X58529, M28170, M31523, M11722, J03473, X03934, U23852, X00437, M23323, X59871, X76223, D00749, L05148, U14603, M37271, M26692, M12886, J05243, X69398, U67171, X04145, L10373, U16954, J04132, M28826, HG4128, X87241, U50743, M13792, L47738, X95735, X17042, M23197, M84526, L09209, U46499, M27891, M16038, M63138, M55150, M22960, M62762, X61587, and U50136, and wherein the level of gene expression of said at least one gene allows classification of a leukemia as AML, B-ALL or T-ALL with a misclassification rate of 40% or lower; comparing the level of gene expression of said at least one gene to a first control level of gene expression of said at least one gene as measured in an AML cell; comparing the level of gene expression of said at least one gene to a second control level of gene expression of said at least one gene as measured in a B-ALL cell; and comparing the level of gene expression of said at least one gene to a third level of gene expression of said at least one gene as measured in a T-ALL cell; wherein the leukemia is classified as AML, B-ALL or T-ALL depending on whether the level of gene expression of the at least one gene is more mathematically similar to the first control level of gene expression; the second control level of gene expression; or the third control level of gene expression. In certain embodiments, the method may comprise determining the expression levels of at least two genes, optionally at least three genes, five genes, ten genes, twenty genes, thirty genes, or at least forty genes. Furthermore, the misclassification rate may optionally be less than 30%, less than 20%, less than 15%, or less than 10%.

In certain aspects the invention relates to a wide range of applications that any of the gene sets in tables 1, 2, and 3 may be used for. In one embodiment the invention relates to methods for identifying a candidate therapeutic agent for the treatment of a hyperproliferative disorder comprising: (a) contacting a hyperproliferative cell with a test therapeutic agent; (b) determining a level of gene expression of a gene in the cell, wherein said gene is selected from the group consisting of Neuromedin U; Aldehyde dehydrogenase 9 (Human gamma-aminobutyraldehyde dehydrogenase E3 isozyme); Fibroblast growth factor 8; Human epidermal growth factor receptor (HER3); Translocase of outer mitochondrial membrane 34; KIAA0089; Monoamine oxidase B; Urokinase plasminogen activator; Zinc finger protein 273; clone 1D2; Aldehyde dehydrogenase 10 (fatty aldehyde dehydrogenase); Carboxylesterase 2 (intestine, liver); Gro2 oncogene; Diazepam binding inhibitor; Cadherin 17; TAL1 (SCL) interrupting locus; Crystallin alpha B; 5T4 oncofetal trophoblast glycoprotein; Deoxyribonuclease I-like 3; Heat-shock protein 90-kDa; Smg GDS-associated protein; Cytochrome c oxidase subunit Vb (coxVb); Wilm Tumor-Related Protein; TYRO3 protein tyrosine kinase; FAT tumor suppressor; Creatine kinase, mitochondrial 1; Ferritin, light polypeptide; Transcription factor 20; MHC class I polypeptide related sequence A; KIAA0018 gene product 1; Lectin galactoside-binding, soluble, 7 (galectin 7); Tenascin-R (restrictin, janusin); CD1A antigen, a polypeptide; Cytochrome P4502C9 subfamily IIC (mephytoin4-hydroxylase), polypeptide 9; Phospholipase A2, group VII; Beta-Hexosaminidase, Alpha Polypeptide, Abnormal Splice Mutation; clone 1A7; KIAA0172 gene; Interleukin 8 receptor, beta; Myxovirus (influenza) resistance 2, homolog of murine; Lysophospholipase like; Interleukin-8 receptor type B, splice variant IL8RB9; keratin 4; Runt-related transcription factor; and Cathepsin L; and wherein the level of gene expression of said gene allows classification of an oral keratinocyte as hyperproliferative or non-hyperproliferative with a misclassification rate of 40% or lower; and determining whether the expression level of said gene is more mathematically similar to that of a proliferative cell or a non-hyperproliferative cell, wherein a test therapeutic agent that causes the expression level of the gene in the hyperproliferative cell to more closely resemble the expression level of the gene in a non-hyperproliferative cell is a candidate therapeutic agent. In certain embodiments, the method may comprise determining the expression levels of at least two genes, optionally at least three genes, five genes, ten genes, twenty genes, thirty genes, or at least forty genes. Furthermore, the misclassification rate may optionally be less than 30%, less than 20%, less than 15%, or less than 10%.

### BRIEF DESCRIPTION OF THE DRAWINGS

FIG. 1: depicts a comparison of three idealized gene expression distributions.

FIG. 2: a) depicts the top 200 discriminating genes rank ordered by the F statistic which provides a conservative estimate of the genes characteristic of the cancerous state; b) depicts cross-validation results of classification through the FDA analysis to determine discriminatory genes.

FIG. 3: depicts the FDA method of a 2-D projection (along axes FV1 and FV2) of the expression data of three genes such that the separation of the three sample classes is maximized.

FIG. 4: depicts the projection of the expression phenotypes of cultures of Synechocystis sp. PCC 6803 to a FDA-defined space.

FIG. 5: depicts a) a schematic diagram for the leave one out cross-validation (LOOCV) algorithm; and b) a schematic diagram for the power analysis algorithm for determination of the minimum sample size.

FIG. 6: depicts the determination of minimum sample size for two-class (ALL, AML) distinction, selection of discriminatory genes with the estimated sample sizes of two classes, and FDA projection.

FIG. 7: depicts the determination of minimum sample size for the three-class (B-ALL, T-ALL, AML) distinction, selection of discriminatory genes with the estimated sample sizes of three classes, and FDA projection.

FIG. 8: depicts the physiological state domains defined by a series of decision boundaries for the projected values in the reduced CV plane.

FIG. 9: depicts an FDA projection of 27 yeast deletion mutant expression phenotype experiments grouped b the functionality of the eliminated gene.

FIG. 10: depicts the FDA results using 45 genes. The projected value (FDA scores) into CV were calculated using linear combination of individual gene expressions, CV=v₁g₁+v₂g₂+ . . . +V₄₅g₄₅. a) depicts the top 45 discriminatory genes, b) 2000^{th}-2045^{th} genes, c) 4000^{th}-4045^{th} genes, d) 6000^{th}-6045^{th} genes. The overlap and variation in the groups increases when genes are chosen poorly.

FIG. 11: depicts an FDA projection of expression data obtained from patients with B-ALL, T-ALL, and AML.

FIG. 12: depicts an FDA projection of the expression phenotypes comprising 7070 genes measured in samples obtained from healthy individuals (5 samples) and patients with oral epithelium cancer (5 samples).

FIG. 13: depicts a full-genome DNA micro-array for Synechocystis sp. PCC6803.

FIG. 14: depicts the results for the FDA of DNA micro-array results for all samples; a) the resulting projection values, CV1 and CV2, are displayed along with PHB accumulation level; b) 2-D representation of a) where PHB accumulation level has been removed; c) correlation between CV2 and PHB accumulation values was significant across the 23 conditions evaluated.

FIG. 15: depicts plots of transcript accumulation levels for a) a representative mix of the top 30 discriminating genes, b) phosphate related genes, and c) ntrogen related genes.

FIG. 16: depicts transcript accumulation levels for PHB biosynthetic genes as described in reaction (A). PHB accumulation levels for the same conditions. PhaEC transcript accumulation level very closely followed PHB acumulation levels. Both phaAB and phaEC are bicistronic and good agreement between there values was observed.

### DETAILED DESCRIPTION

1. Definitions

“Amplification of polynucleotides” utilizes methods such as the polymerase chain reaction (PCR), ligation amplification (or ligase chain reaction, LCR) and amplification methods based on the use of Q-beta replicase. These methods are well known and widely practiced in the art. Reagents and hardware for conducting PCR are commercially available. Primers useful to amplify sequences from HPE genes are preferably complementary to, and hybridize specifically to sequences in the HPE coding sequences, introns, or in flanking regions of the mRNA. HPE

“Analyte polynucleotide” and “analyte strand” refer to a single- or double-stranded polynucleotide which is suspected of containing a target sequence, e.g., an HPE seequence, and which may be present in a variety of types of samples, including biological samples.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, or other purified preparations of antibodies and recombinant antibodies.

A “between group measure of variability”, denoted “B” in some embodiments, includes measures of the difference between the total variability (sometimes referred to as “T”) and the within group variability (sometimes referred to as “W”). For example, B may be calculated as T-W, but other mathematical methods for calculating the difference may be employed.

A “biological sample” refers to a sample of tissue or fluid suspected of containing an analyte polynucleotide or polypeptide from an individual including, but not limited to, e.g., plasma, serum, spinal fluid, lymph fluid, the external sections of the skin, respiratory, intestinal, and genitourinary tracts, tears, saliva, blood cells, tumors, organs, tissue and samples of in vitro cell culture constituents. A “sample” refers generally to any material suspected of containing an analyte polynucleotide or polypeptide of interest, including but not limited to samples from a cell culture, samples from an animal or plant, etc.

The term “carcinoma” refers to a malignant new growth made up of epithelial cells tending to infiltrate surrounding tissues and to give rise to metastases. Exemplary carcinomas include: “basal cell carcinoma”, which is an epithelial tumor of the skin that, while seldom metastasizing, has potentialities for local invasion and destruction; “squamous cell carcinoma”, which refers to carcinomas arising from squamous epithelium and having cuboid cells; “carcinosarcoma”, which include malignant tumors composed of carcinomatous and sarcomatous tissues; “adenocystic carcinoma”, carcinoma marked by cylinders or bands of hyaline or mucinous stroma separated or surrounded by nests or cords of small epithelial cells, occurring in the mammary and salivary glands, and mucous glands of the respiratory tract; “epidermoid carcinoma”, which refers to cancerous cells which tend to differentiate in the same way as those of the epidermis; i.e., they tend to form prickle cells and undergo cornification; “nasopharyngeal carcinoma”, which refers to a malignant tumor arising in the epithelial lining of the space behind the nose; and “renal cell carcinoma”, which pertains to carcinoma of the renal parenchyma composed of tubular cells in varying arrangements. Another carcinomatous epithelial growth is “papillomas”, which refers to benign tumors derived from epithelium and having a papillomavirus as a causative agent; and “epidermoidomas”, which refers to a cerebral or meningeal tumor formed by inclusion of ectodermal elements at the time of closure of the neural groove.

The terms “complementary” or “complementarity”, as used herein, refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between the single stranded molecules. The degree of complementarity between nucleic acid strands has significant effects on the efficiency and strength of hybridization between nucleic acid strands. This is of particular importance in amplification reactions, which depend upon binding between nucleic acids strands and in the design and use of PNA molecules.

The term “correlates with expression of a polynucleotide”, as used herein, indicates that the detection of the presence of ribonucleic acid that is similar to one of HPE genes by northern analysis is indicative of the presence of mRNA encoding an HPE gene product in a sample and thereby correlates with expression of the transcript from the polynucleotide encoding the protein.

The term “discriminant function” refers to the function that describes the projection of a data set into the dimensional space of a classification system. The term “discriminant function” or “DF” as used herein is interchangeable with the term “canonical variable” or “CV”.

The term “discriminant loadings” refers to the set of values derived from a set of control data that may be applied to a data set from an unclassified sample in order to project the unclassified sample into the dimensional space of the classification system. The term “discriminant loadings” as used herein is interchangeable with the term “canonical coefficients” and is intended to have an identical meaning.

The term “diagnostic array of HPE gene probes” refers to a set, e.g., at least a minimal set, of HPE genes that will produce statistically significant discrimination between normal and transformed epithelial cells and/or metastatic and non-metastatic epithelial tumor cells. For instance, the diagnostic array of HPE gene probes may include at least a sufficient number of sequences such that, by an error classification model, the probe set achieves a correct classification rate between normal and transformed, or metastatic and non-metastatic, of at least 75 percent, more preferably 80, 85 or even 90 percent. The probe set may be a set of nucleic acid probes that includes at least a sufficient number of probes for the HPE genes that, by Fisher discriminant analysis, the probe set defines a set of canonical coefficients that will produce statistically significant discrimination between normal and transformed epithelial cells or metastatic and non-metastatic epithelial cells. In certain preferred embodiments, the probe set hybridizes to at least 5 HPE genes shown in Table 1, and more preferably at least 10, 20, 30 or 40 HPE genes shown in Table 1.

The term “diagnostic array of HPE binding agents” refers to a set of binding agents, such as antibodies (monoclonal, recombinant, single chain, etc.) which bind to HPE gene products and provide a statistically significant discrimination between normal and transformed or metastatic and non-metastatic epithelial tissues. In certain preferred embodiments, the antibody set includes antibodies for at least 5 HPE proteins shown in Table 1, and more preferably at least 10, 20, 30 or 40 HPE proteins shown in Table 1. In certain preferred embodiments, the antibody array is for detecting secreted HPE proteins.

In either case, the probe set can be provided free in an antibody solution or immobilized on a solid support. For instance, the probe set can be divided up and individual members presented in microlitre wells. In other embodiments, the probe or antibody sets can be spatially arrayed on a glass or other chip format.

The term “discriminant function analysis” refers to the method of finding a transform which gives the maximum ratio of difference between a pair of group multivariate means to the multivariate variance within the two groups. Accordingly, it involved delineation based upon maximizing between group variance while minimizing within group variance.

The phrase “discriminating gene or protein” is used to describe a gene or protein, the expression level of which is useful for determining the classification of a sample.

The terms “epithelia”, “epithelial” and “epithelium” refer to the cellular covering of internal and external body surfaces (cutaneous, mucous and serous), including the glands and other structures derived therefrom, e.g., corneal, esophegeal, epidermal, and hair follicle epithelial cells. Other exemplary epithlelial tissue includes: olfactory epithelium, which is the pseudostratified epithelium lining the olfactory region of the nasal cavity, and containing the receptors for the sense of smell; glandular epithelium, which refers to epithelium composed of secreting cells; squamous epithelium, which refers to epithelium composed of flattened plate-like cells. The term epithelium can also refer to transitional epithelium, which that characteristically found lining hollow organs that are subject to great mechanical change due to contraction and distention, e.g. tissue which represents a transition between stratified squamous and columnar epithelium.

A disease, disorder, or condition “associated with” or “characterized by” an aberrant expression of HPE genes refers to a disease, disorder, or condition in a subject which is caused by, contributed to by, or causative of an aberrant level of expression of a nucleic acid.

The term “Fisher Discriminant Analysis” or “FDA” is used interchangeably herein with the term “Canonical Discriminant Analysis”.

As used herein, the phrase “gene or protein expression information” includes any information pertaining to the amount of gene transcript or protein present in a sample, as well as information about the rate at which genes or proteins are produced or are accumulating or being degraded (eg. reporter gene data, data from nuclear runoff experiments, pulse-chase data etc.). Certain kinds of data might be viewed as relating to both gene and protein expression. For example, protein levels in a cell are reflective of the level of protein as well as the level of transcription, and such data is intended to be included by the phrase “gene or protein expression information”. Such information may be given in the form of amounts per cell, amounts relative to a control gene or protein, in unitless measures, etc.; the term “information” is not to be limited to any particular means of representation and is intended to mean any representation that provides relevant information. The term “expression levels” refers to a quantity reflected in or derivable from the gene or protein expression data, whether the data is directed to gene transcript accumulation or protein accumulation or protein synthesis rates, etc.

The “growth state” of a cell refers to the rate of proliferation of the cell and the state of differentiation of the cell.

The term “hybridization”, as used herein, refers to any process by which a strand of nucleic acid binds with a complementary strand through base pairing.

As used herein, “immortalized cells” refers to cells which have been altered via chemical and/or recombinant means such that the cells have the ability to grow through an indefinite number of divisions in culture.

The term “keratosis” refers to proliferative skin disorder characterized by hyperplasia of the horny layer of the epidermis. Exemplary keratotic disorders include keratosis follicularis, keratosis palmaris et plantaris, keratosis pharyngea, keratosis pilaris, and actinic keratosis.

The term “mathematically similar” is intended to include any of the various quantitative methods for determining the similarity between sets of data. Such methods might include calculations of Euclidean distance or correlation coefficients. In addition, methods involving determining a measure of variability as described herein are methods of determining mathematical similarity. The term “mathematical similarity” is also intended to include measures of mathematical dissimilarity when the two measures contain essentially the same information.

A “matrix”, as used in reference to mathematical methods, includes any representation of information that is amenable to the methods of linear algebra, whether or not the information is represented as a set of columns and rows.

A “measure of variability” is any measure of variation in data, including but not limited to range, standard deviation, kurtosis and variance, as well as any simple transformation of the foregoing, such as multiplication, division, inverse, root, exponent, log, etc.

A “metabolite” as used herein includes anything produced by a cell, whether it is a natural product of the cell or whether the cell has been manipulated to cause production of the metabolite.

“Microarray” refers to an array of distinct polynucleotides or oligonucleotides synthesized on a substrate, such as paper, nylon or other type of membrane, filter, chip, glass slide, or any other suitable solid support.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

An “orthologue” of a gene is the equivalent of that gene in another species. An ortholog is conserved at the sequence level (usually greater than 50% identity, sometimes 60%, 70%, or even 80% or greater) and is also conserved at the functional level.

A “patient” or “subject” to be treated by the subject method can mean either a human or non-human animal.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. Various alignment algorithms and/or programs may be used, including FASTA, BLAST or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

As used herein, “phenotype” refers to the entire physical, biochemical, and physiological makeup of a cell, e.g., having any one trait or any group of traits.

A “polyhydroxyalkanoic acid” is intended to include any member of that polymer class, regardless of ionization state, side groups, branching, mixing of subunit types, etc. As used herein, “polyhydroxyalkanoic acid” is used interchangeably with “polyhyroxyalkanoate”. Similarly, “polyhydroxybutyric acid”, as an example of a polyhydroxyalkanoic acid, is used interchangeably with “polyhydroxybutyrate”, and the same applies to other members of the class.

The terms “protein”, “polypeptide” and “peptide” are used interchangeably herein.

As used herein, “proliferating” and “proliferation” refer to cells undergoing mitosis.

Throughout this application, the term “proliferative skin disorder” refers to any disease/disorder of the skin marked by unwanted or aberrant proliferation of cutaneous tissue. These conditions are typically characterized by epidermal cell proliferation or incomplete cell differentiation, and include, for example, X-linked ichthyosis, psoriasis, atopic dermatitis, allergic contact dermatitis, epidermolytic hyperkeratosis, and seborrheic dermatitis. For example, epidermodysplasia is a form of faulty development of the epidermis. Another example is “epidermolysis”, which refers to a loosened state of the epidermis with formation of blebs and bullae either spontaneously or at the site of trauma.

As used herein, the term “psoriasis” refers to a hyperproliferative skin disorder which alters the skin's regulatory mechanisms. In particular, lesions are formed which involve primary and secondary alterations in epidermal proliferation, inflammatory responses of the skin, and an expression of regulatory molecules such as lymphokines and inflammatory factors. Psoriatic skin is morphologically characterized by an increased turnover of epidermal cells, thickened epidermis, abnormal keratinization, inflammatory cell infiltrates into the dermis layer and polymorphonuclear leukocyte infiltration into the epidermis layer resulting in an increase in the basal cell cycle. Additionally, hyperkeratotic and parakeratotic cells are present.

The term “substantially homologous”, when used in connection with amino acid sequences, refers to sequences which are substantially identical to or similar in sequence, giving rise to a homology in conformation and thus to similar biological activity. The term is not intended to imply a common evolution of the sequences.

As used herein, “transformed cells” refers to cells which have spontaneously converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control.

2. Overview

There are several research issues that may be addressed with gene and protein expression data, each requiring a particular set of bioinformatic tools. Commonly asked questions include: (a) Of the large number of genes probed, which ones are particularly relevant to a disease or, in general, a cellular state of interest, by virtue of their ability to characterize a particular cellular state as such; (b) Is there a specific pattern of gene expression that marks the occurrence of a particular physiological state; and (c) Can such patterns be used to diagnose the physiological state of cell and tissue samples?. Although some answers to the above questions can be obtained by simple visual inspection of a sample's expression levels relative to those of the control, statistical significance is increased by applying rigorous analysis in identifying discriminatory genes and their characteristic patterns.

In one aspect, the present invention relates to novel methods for analyzing gene and protein expression data. In one embodiment, the invention provides methods for identifying, in data from a sample of a classified cellular state, genes or proteins that are relevant to the cellular state and/or to differences between two or more cellular states. In some embodiments, genes or proteins identified in this manner may form the basis for new research programs directed towards understanding the role of a gene or protein in a particular cellular state. In other embodiments, the expression of genes or proteins identified in this way may be used to classify a sample, inlcuding, for example, diagnosing a biopsy for the presence of a disease state. In yet further embodiments, the expression of relevant genes and proteins may be manipulated in an effort to create a desired cellular state, such as, for example, returning hyperproliferative cells to a normal state or engineering bacteria to enhance production of a desirable compound.

Novel methods for analyzing gene and protein expression data provided herein may also be used, in certain embodiments, to generate classes of genes or proteins that are related to certain cellular states. This information may highlight previously unappreciated relationships between genes, and may also provide functional information about genes with no previously known function. This aspect of the invention is particularly important in view of the exponentially increasing wealth of sequence data. In some embodiments, the operation of analytical methods described herein defines a gene or protein expression pattern or patterns that are diagnostic of a certain cellular state. Such patterns provide substantial power for the classification of samples and other purposes described above with respect to single genes.

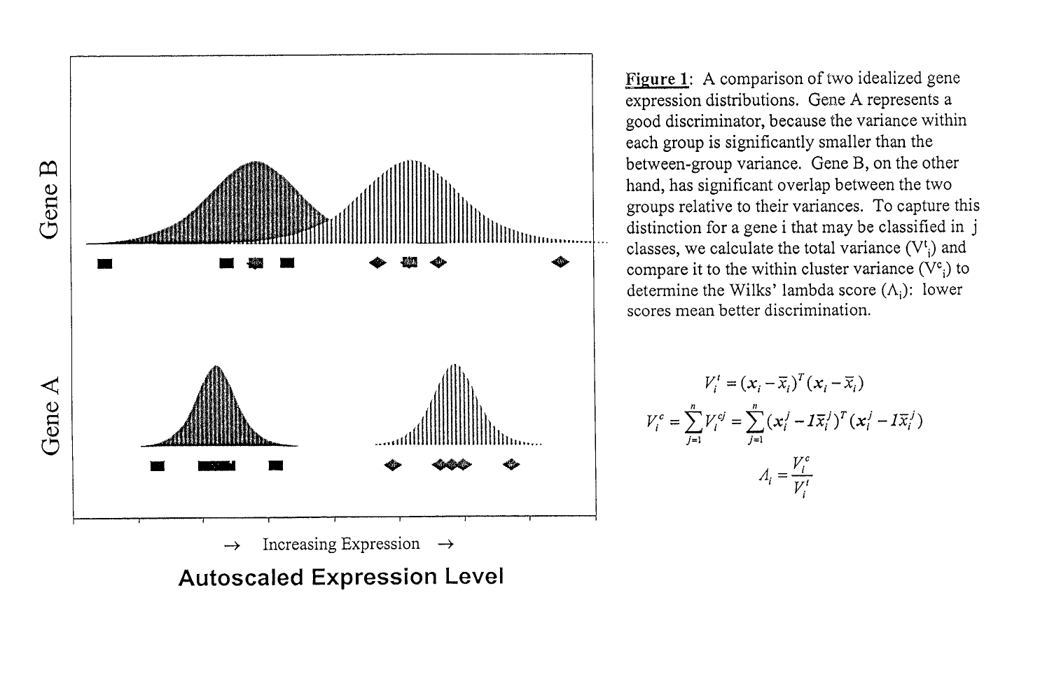

In general, it is expected that only a subset of the total number of genes or proteins whose expression is measured will be of consequence in distinguishing a physiological state of interest. This is shown schematically in FIG. 1 depicting the expression distribution of two genes A and B in ten samples obtained from two different types (or classes) of tissues or cells, such as, for example, normal and diseased or different classes of disease. Clearly, while the expression of gene A is sufficiently distinct in the two types, the significant overlap in the expression of gene B for the two classes of samples reduces its value in differentiating one class of tissue or cell from another.

Other methods for analyzing gene expression data have tended to be based on parametric distributions. When a criterion for selection of discriminatory variables is based on a parametric distribution (e.g. a t-test), the two assumptions of normality and equal variance in each class should be met to ensure the optimal performance of the test. Thomas et al. (2001) have compared various criteria that can be used for selection of discriminatory genes in two classes and showed how a violation of these assumptions can affect the identity of discriminatory genes identified by those techniques. A standard Q-Q plot comparing data to a normal distribution can be performed to check the expression level of a gene across the samples. Additionally, a hypothesis test (Ho: s₁=s₂; H₁: s₁₁≠s2) or Bartlett's test in the multivariate case can be preformed to see whether each class has the same variance. Johnson, R. A. & Wichern, D. W. Applied Multivariate Statistical Analysis. (Prentice Hall, N.J., 1992). There exists a body of literature discussing how badly these assumptions can be violated for practical applications and how violations can affect the robustness of the test (e.g. increased false positive error in the t-test), depending upon sample sizes in classes. Holloway, L. N., & Dunn, O. The robustness of Hotelling's T². Journal of the American Statistical Association 62, 124-136 (1967). Johnson and Wichern (1992) suggested that any discrepancy where one variance is four times larger than the other (s₁=4s₂, or vice versa) could pose serious classification problems. Several methods, such as a modified t-test using effective degrees of freedom, have been proposed to achieve the optimal test performance in the case that the data violate the assumption of equal variance. Welch, B. L. The generalization of Student's problem when several populations are involved. Biometrika 34, 28-35, (1947).

In some embodiments, the present invention relates to methods of analysis that are not based on parametric distributions. For example, certain methods described herein allow analysis of gene and protein expression data through the analysis of a measure of variability.

As presented below, the analytical methods described herein are applicable to problems as diverse as the diagnosis of hyperproliferative cells, the classification of neoplasms, and the production of useful compounds by bacteria. Accordingly, in certain aspects, the invention provides genes, genes groups and patterns that are related to hyperproliferative states of epithelial cells (See Table 1), the distinction between forms of leukemias (See Table 2) In addition to identifying disease states such as oral cancer, use of projections such as those defined by Fisher Discriminant Analysis (FDA) in defining cellular physiological states from gene or protein expression measurements can also be used to systematically probe environmental conditions and the effects these conditions have on the cell's physiology. As described in the examples below, full genome DNA micro-arrays may now be used to profile transcriptional alterations in Synechocystis cells that have accumulated different levels of the biopolymer polyhydroxybutyrate (PHB) under varying nutritional conditions will help to develop metabolic engineering approaches to improve PHB accumulation. In certain embodiments, the invention provides genes, gene groups and patterns of gene expression that are related to the production of polyhydroxyalkanoates (See Table 3).

In some embodiments, such information may be used to classify other samples. For example, a sample from a subject may be used to generate gene and/or protein expression data. Such data may then be compared against the genes or patterns described herein to assess the hyperproliferative state of cells in the sample. Similarly, a culture of bacteria may be sampled to help assess the likely polyhydroxyalknaote production. In making this type of comparison, it is not necessary to employ the novel analytical methods described herein. Such comparisons may be accomplished by any of the various methods that are, in view of this specification, known in the art.

In its various aspects and embodiments, the invention includes providing a test cell population, such as from a tissue biopsy. Expression of one, some, or all of the genes identified herein is detected, if present, and, preferably, measured. Using the information provided by the database entries for the known genes, or the information provided herein for the previously unknown genes, the expression of the such sequences can be detected, if present, and measured using techniques well known to one of ordinary skill in the art. For example, sequences within public sequence database entries for the HPE sequences or within the novel sequences disclosed herein can be used to construct probes for detecting HPE RNA sequences in, for example, northern blot hybridization analyses, RT-PCR analyses, etc. Alternatively, the sequences can be used to construct primers for specifically amplifying the HPE sequences in, for example, amplification-based detection methods such as reverse transcription-based polymerase chain reaction (PCR).

The expression level(s) of one or more of the identified gene in the test cell population may then be compared to expression levels of the sequences in one or more cells from a reference cell population. A reference cell population includes one or more cells for which the compared parameter or condition is known. The composition of the reference cell population will determine whether the comparison of gene expression profile indicates the presence or absence of the measured parameter or condition.

An alteration of the expression in the test cell population, as compared to the reference cell population, indicates that the measured parameter or condition in the test cell population is different than that of the reference cell. The absence of the alteration of expression in the test cell population, as compared to the reference cell population, indicates that the measured parameter or condition in the test cell population is the same as that of the reference cell. As an example, if the reference cell population contains noncancerous cells, a similar gene expression profile in the test cell population indicates that the test cells are also non-cancerous, whereas a different profile indicates that the test cells are cancerous. Likewise, if the reference cell population is made up of cancerous cells, a similar expression profile in the test cell population indicates that the test cell population also includes cancerous cells, and a different expression profile indicates that test cells are noncancerous.

As described below, one embodiment provides 45 genes that are discovered to be strongly correlated with epithelial cancer, and particularly oral tumor malignancy. The elevated expression of three of these genes was further confirmed by real-time quantitative PCR of the original samples as well as samples from five new pairs of cases. Of the 45 genes identified, 6 have been previously implicated in the disease, and 2 are uncharacterized clones. The present invention provides the ability to analyze changes in the levels of the transcripts and/or protein products for multiple different genes in oral or other epithelial tissue.

The method includes obtaining a biopsy, which is optionally fractionated by cryostat sectioning to enrich tumor cells, e.g., to at least 80% of the total cell population. The DNA or RNA is then extracted, amplified, and analyzed with a DNA chip to determine the presence of absence of the marker nucleic acid sequences.

In some embodiments, the test cell population is compared to multiple reference cell populations. Each of the multiple reference cell populations might differ in the known parameter. For example, a test cell population may be compared to a reference cell population containing normal epithelial cells, as well as other reference cell populations known to contain metastatic cancerous cells.

The test cell population may be known to contain or be suspected of containing a neoplasm. In some embodiments, the test cell will be included in a cell sample known to contain or suspected of containing transformed or hyper-proliferative epithelial cells, such as cells from an epithelial carcinoma. Preferably, cells in the reference cell population are derived from a tissue type that is as similar to the test cell population as possible. For example, the reference cell population may be derived from similar epithelial tissue, e.g., oral, breast, gastrointestinal, etc. In some embodiments, the reference cell is derived from a region proximal to the region of origin of the test cell population.

In some embodiments, the reference cell population is derived from a plurality of cells. The reference cell population can be a database of expression patterns from previously tested cells for which one of the assayed parameters or conditions is known.

In various embodiments, the expression of all, or at least a diagnostic array of the sequences represented in Tables 1, 2 or 3 are measured.

TABLE 1
List of 45 discriminatory genes of oral epithelium cancer.

| | | Up/Down | | | | |
|---|---|---|---|---|---|---|
| Accession | | Regulated in | Oral Cancer | Chromosome | | |
| Number | Gene Name | Cancer | Association | Location | Function | Significance |
| X76029 | Neuromedin U | Down in cancer | | 4q12 | | unexpected |
| | | | | | | finding |
| U34252 | Aldehyde | Down in cancer | 1q22-q23 | xenobiotic | | |
| | dehydrogenase 9 | | | metabolism | | |
| | (Human gamma- | | | | | |
| | aminobutyraldehyde | | | | | |
| | dehydrogenase E3 | | | | | |
| | isozyme) | | | | | |
| U47011 | Fibroblast growth | Down in cancer | | 10q24 | oncogene | opposite result |
| | factor 8 | | | | | than expected |
| M34309 | Human epidermal | Down in cancer | | 12q13 | | opposite result |
| | growth factor | | | | | than expected |
| | receptor (HER3) | | | | | |
| U58970 | Translocase of | Up in cancer | | 20 | | mitochondrial |
| | outer mitochondrial | | | | | protein import |
| | membrane 34 | | | | | |
| D42047 | KIAA0089 | Down in cancer | | 3 | Related to mouse | |
| | | | | | glycerophosphate | |
| | | | | | dehydrogenase | |
| M69177 | Monoamine oxidase | Down in cancer | | Xp11.4-p11.3 | | |
| | B | | | | | |
| X02419 | Urokinase | Up in cancer | + | 10q24 | Biomarker | Invasion |
| | plasminogen | | | | | pathway |
| | activator | | | | | |
| X78932 | Zinc finger protein | Down in cancer | | N/A | Transcription | |
| | 273 | | | | factor | |
| Z78289 | clone 1D2 | Down in cancer | | N/A | | |
| U46689 | Aldehyde | Down in cancer | | 17p11.2 | Xenobiotic | |
| | dehydrogenase 10 | | | | metabolism | |
| | (fatty aldehyde | | | | | |
| | dehydrogenase) | | | | | |
| Y09616 | Carboxylesterase 2 | Down in cancer | | 16 | xenobiotic | |
| | (intestine, liver) | | | | metabolism | |
| M57731 | Gro2 oncogene | Up in cancer | | 4q21 | 90% identical to | |
| | | | | | Gro1 | |
| M14200 | Diazepam binding | Down in cancer | | 2q12-q21 | | |
| | inhibitor | | | | | |
| U07969 | Cadherin 17 | Down in cancer | | 8q22.2-q22.3 | Cadherin family | In a |
| | | | | | | chromosomal |
| | | | | | | location where |
| | | | | | | LOH is present |
| M74558 | TAL1 (SCL) | Up in cancer | | 1q32 | SCL interrupting | leukemia |
| | interrupting locus | | | | locus | associated |
| | | | | | | gene |
| S45630 | Crystallin alpha B | Down in cancer | | 11q22.3-q23.1 | molecular | small heat |
| | | | | | chaperone | shock protein |
| | | | | | activity | |
| Z29083 | 5T4 oncofetal | Up in cancer | | 6 | Metastasis | contributes to |
| | trophoblast | | | | | the process of |
| | glycoprotein | | | | | placentation or |
| | | | | | | metastasis by |
| | | | | | | modulating cell |
| | | | | | | adhesion, |
| | | | | | | shape and |
| | | | | | | motility |
| U56814 | Deoxyribonuclease | Down in cancer | | 3p21.1-3p14.3 | apoptosis related | |
| | 1-like 3 | | | | | |
| X15183 | Heat-shock protein | Up in cancer | | 1q21.2-q22 | | |
| | 90-kDa | | | | | |
| U59919 | Smg GDS- | Up in cancer | | 1 | phosphorylated | signal |
| | associated protein | | | | by v-src | transduction |
| | | | | | | pathway |
| M19961 | Cytochrome c | Down in cancer | | 2cen-q13 | | |
| | oxidase subunit Vb | | | | | |
| | (coxVb) | | | | | |
| HG3549- | Wilm Tumor- | Down in cancer | | N/A | | |
| HT3751 | Related Protein | | | | | |
| U18934 | TYRO3 protein | Down in cancer | | 15q15.1-q21.1 | | |
| | tyrosine kinase | | | | | |
| X87241 | FAT tumor | Up in cancer | | 4q34-q35 | tumor suppressor | opposite results |
| | suppressor | | | | | |
| J04469 | Creatine kinase, | Down in cancer | | 15q15 | | |
| | mitochondrial 1 | | | | | |
| M11147 | Ferritin, light | Up in cancer | + | 19q13.3-q13.4 | biomarker | |
| | polypeptide | | | | | |
| U19345 | Transcription factor | Down in cancer | | 22q13.2-q13.3 | Metastatic | controls |
| | 20 | | | | pathway | stromelysin |
| | | | | | | expression |
| L14848 | MHC class I | Down in cancer | | 6p21.3 | | |
| | polypeptide related | | | | | |
| | sequence A | | | | | |
| D13643 | KIAA0018 gene | Down in cancer | | 1 | | |
| | product 1 | | | | | |
| U06643 | Lectin galactoside- | Down in cancer | | 19 | role in cell-cell | |
| | binding, soluble, 7 | | | | and/or cell-matrix | |
| | (galectin 7) | | | | interactions | |
| | | | | | necessary for | |
| | | | | | normal growth | |
| | | | | | control | |
| X98085 | Tenascin-R | Down in cancer | | 1q24 | contains EGF-like | |
| | (restrictin, janusin) | | | | repeats | |
| M28825 | CD1A antigen, a | Down in cancer | | 1q22-q23 | | |
| | polypeptide | | | | | |
| M61855 | Cytochrome | Down in cancer | + | 10q24 | xenobiotic | |
| | P4502C9 subfamily | | | | metabolism | |
| | IIC (mephytoin4- | | | | | |
| | hydroxylase), | | | | | |
| | polypeptide 9 | | | | | |
| U24577 | Phospholipase A2, | Up in cancer | + | 6p21.2-p12 | | |
| | group VII | | | | | |
| HG2992- | Beta- | Up in cancer | | N/A | | |
| HT5186 | Hexosaminidase, | | | | | |
| | Alpha Polypeptide, | | | | | |
| | Abnormal Splice | | | | | |
| | Mutation | | | | | |
| Z78285 | clone 1A7 | Up in cancer | | N/A | | |
| D79994 | KIAA0172 gene | Down in cancer | | 9 | | |
| L19593 | Interleukin 8 | Down in cancer | + | 2q35 | | |
| | receptor, beta | | | | | |
| M30818 | Myxovirus | Up in cancer | | 21q22.3 | | |
| | (influenza) resistance | | | | | |
| | 2, homolog of murine | | | | | |
| U67963 | Lysophospholipase | Down in cancer | | 3 | | |
| | like | | | | | |
| U11877 | Interleukin-8 | Down in cancer | | | | |
| | receptor type B, | | | | | |
| | splice variant | | | | | |
| | IL8RB9 | | | | | |
| X07695 | keratin 4 | Down in cancer | | 12q13 | | |
| D43968 | Runt-related | Up in cancer | | 21q22.3 | transcription | |
| | transcription factor | | | | factor | |
| X12451 | Cathepsin L | Up in cancer | + | 9q21-q22 | Metastasis | |

TABLE 2
List of 48 exemplary discriminatory genes of ALL and AML leukemia.

| Gene ID | Gene Description |
|---|---|
| U05259* | MB-1 gene |
| M89957 | IGB Immunoglobulin-associated beta (B29) |
| M84371 | IGB Immunoglobulin-associated beta (B29) |
| D88270 | GB DEF = (lambda) DNA for immunoglobin light chain |
| X58529 | IGHM Immunoglobulin mu |
| M28170 | CD19 CD19 antigen |
| M31523* | TCF3 Transcription factor 3 (E2A immunoglobulin enhancer |
| | binding factors E12/E47) |
| M11722 | Terminal transferase mRNA |
| JO3473 | ADPRT ADP-ribosyltransferase (NAD+; poly (ADP-ribose) |
| | polymerase) |
| X03934 | GB DEF = T-cell antigen receptor gene T3-delta |
| U23852 | GB DEF = T-lymphocyte specific protein tyrosine kinase |
| | p56lck (lck) abberant mRNA |
| X00437 | TCRB T-cell receptor, beta cluster |
| M23323 | T-CELL SURFACE GLYCOPROTEIN CD3 EPSILON |
| | CHAIN PRECURSOR |
| X59871 | TCF7 Transcription factor 7 (T-cell specific) |
| X76223 | GB DEF = MAL gene exon 4 |
| D00749 | T-CELL ANTIGEN CD7 PRECURSOR |
| L05148 | Protein tyrosine kinase related mRNA sequence |
| U14603 | Protein tyrosine phosphatase PTPCAAX2 (hPTPCAAX2) |
| | mRNA |
| M37271 | T-CELL ANTIGEN CD7 PRECURSOR |
| M26692 | GB DEF = Lymphocyte-specific protein tyrosine kinase |
| | (LCK) gene, exon 1, and downstream promoter region |
| M12886 | TCRB T-cell receptor, beta cluster |
| J05243 | SPTAN1 Spectrin, alpha, non-erythrocytic 1 (alpha-fodrin) |
| X69398 | CD47 CD47 antigen (Rh-related antigen, integrin-associated |
| | signal transducer) |
| U67171 | GB DEF = Selenoprotein W (selW) mRNA |
| X04145 | CD3G CD3G antigen, gamma polypeptide (TiT3 complex) |
| L10373 | MXS1 Membrane component, X chromosome, surface |
| | marker 1 |
| U16954 | (AF1q) mRNA |
| J04132 | CD3Z CD3Z antigen, zeta polypeptide (TiT3 complex) |
| M28826 | CD1B CD1b antigen (thymocyte antigen) |
| HG4128 | Anion Exchanger 3, Cardiac Isoform |
| X87241 | HFat protein |
| U50743 | Na, K-ATPase gamma subunit mRNA |
| M13792* | ADA Adenosine deaminase |
| L47738* | Inducible protein mRNA |
| X95735* | Zyxin |
| X17042* | PRG1 Proteoglycan 1, secretory granule |
| M23197* | CD33 CD33 antigen (differentiation antigen) |
| M84526* | DF D component of complement (adipsin) |
| L09209 | APLP2 Amyloid beta (A4) precursor-like protein 2 |
| U46499 | GLUTATHIONE S-TRANSFERASE, MICROSOMAL |
| M27891* | CST3 Cystatin C (amyloid angiopathy and cerebral |
| | hemorrhage) |
| M16038* | LYN V-yes-1 Yamaguchi sarcoma viral related oncogene |
| | homolog |
| M63138* | CTSD Cathepsin D (lysosomal aspartyl protease) |
| M55150* | FAH Fumarylacetoacetate |
| M22960 | PPGB Protective protein for beta-galactosidase |
| | (galactosialidosis) |
| M62762* | ATP6C Vacuolar H+ ATPase proton channel subunit |
| X61587 | ARHG Ras homolog gene family, member G (rho G) |
| U50136* | Leukotriene C4 synthase (LTC4S) gene |

*Genes previously reported. Golub, T. R., D. K. Slonim, P. Tamayo, C. Huard, M. Gaasenbeek, J. P. Meslrov, H. Coller, M. L. Loh, J. R. Downing, M. A. Caligiurl, C. D. Bloomfield and E. S. Lander, “Molecular classification of cancer: Class discovery and class prediction by gene expression monitoring.” Science, 286, 531-537, (1999).

TABLE 3
The preferred thirty exemplary discriminatory genes as determined
by Fisher discriminatory analysis (FDA). Unique ID and Function-Gene
Category are identical to what is contained at Cyanobase. Condition
corresponds to the media growth conditions in which the particular gene
was most significantly altered compared to all of the conditions studied.

| Gene | Unique ID | Function-Gene Category | Condition |
|---|---|---|---|
| Not yet named | sll0008 | None | PA |
| Not yet named | sll0010 | None | N |
| CheY | sll0039 | Chemotaxis protein Y | NA |
| HypF | sll0322 | Transcriptional regulator | BG |
| Not yet named | sll0361 | None | BG |
| ProA | sll0373 | Amino Acid Biosynthesis gG | N |
| Not yet named | sll0374 | Branched chain AA transporter, | N |
| | | braG, livF, livG | |
| Not yet named | sll0379 | Cell envelope, surface | BGA |
| | | polysaccharides | |
| Not yet named | sll0385 | Transport and binding proteins | BG |
| Not yet named | sll0396 | Regulatory components of | BG |
| | | sensory transduction | |
| UvrB | sll0459 | DNA modification and repair, | BG |
| | | cell stress | |
| PrsA | sll0469 | Ribose-phosphate | BG |
| | | pyrophosphokinase | |
| Not yet named | sll0477 | None | BG |
| Not yet named | sll0486 | None | PA |
| Not yet named | sll0550 | Hypothetical flavoprotein | N |
| Not yet named | sll0558 | None | N |
| Not yet named | sll0703 | None | NA |
| NapC | sll0873 | Carboxynorspermidine | NA |
| | | decarboxylase | |
| PetF | sll1317 | Apocytochrome f, | BG |
| | | Photosynthesis | |
| Not yet named | sll1376 | None | BG |
| Not yet named | sll1473 | None | BG |
| Not yet named | sll1504 | None | BGA |
| hsp17 | sll1514 | Heat shock chaperone, | P |
| | | Cell stress | |
| Not yet named | sll1611 | None | NA |
| Not yet named | sll1623 | Transport and binding proteins | NA |
| Not yet named | sll1630 | None | NA |
| Not yet named | sll1632 | None | NA |
| Not yet named | sll1702 | Hypothetical | NA |
| TruA | sll1820 | Pseudouridine synthase I, | BG |
| | | Translation | |
| Nth | slr1822 | Endonuclease m, DNA repair | NA |
| | | and modification | |

If desired, expression of these sequences can be measured along with other sequences whose expression is known to be altered according to one of the herein described parameters or conditions. By “altered’ is meant that the expression of one or more nucleic acid sequences is either increased or decreased as compared to the expression levels in the reference cell population. Alternatively, the expression profile of the test cell population may be the same as that of the reference cell population.

The subject invention provides a method of determining whether a cell sample obtained from a subject possesses an abnormal amount of marker polypeptide which comprises (a) obtaining a cell sample from the subject, (b) quantitatively determining the amount of the marker polypeptide in the sample so obtained, and (c) comparing the amount of the marker polypeptide so determined with a known standard, so as to thereby determine whether the cell sample obtained from the subject possesses an abnormal amount of the marker polypeptide.

Moreover, the represent invention provides a means for understanding the molecular mechanisms underlying transformation of epithelia, as well as for providing reagents and kits for diagnostic and prognostic applications.

3. Gene Expression Data

In general, gene expression data may be gathered in any way that, in view of this specification, is available to one of skill in the art. Although many methods provided herein are powerful tools for the analysis of data obtained by highly parallel data collection systems, many such methods are equally useful for the analysis of data gathered by more traditional methods.

Many gene expression detection methodologies employ a polynucleotide probe which forms a stable hybrid with that of the target gene. If it is expected that the probes will be perfectly complementary to the target sequence, stringent conditions will often provide superior results. Lesser hybridization stringency may be used if some mismatching is expected, for example, if variants are expected with the result that the probe will not be completely complementary. Conditions may be chosen which rule out nonspecific/adventitious bindings, that is, which minimize noise.

Probes for gene sequences may be derived from the sequences of the genomic gene locus or a cDNA or RNA product thereof. The probes may be of any suitable length, which span all or a portion of the RNA sequence, and which allow specific hybridization to transcript (or complementary derivatives thereof, such as cDNAs). If the target sequence contains a sequence identical to that of the probe, the probes may be short, e.g., in the range of about 8-30 base pairs, since the hybrid will be relatively stable under even stringent conditions. If some degree of mismatch is expected with the probe, i.e., if it is suspected that the probe will hybridize to a variant region, a longer probe may be employed which hybridizes to the target sequence with the requisite specificity.

The probes may be an isolated polynucleotide attached to a label or reporter molecule and may be used to isolate other polynucleotide sequences, having sequence similarity by standard methods. For techniques for preparing and labeling probes see, e.g., Sambrook et al., supra, or Ausubel et al., supra. Alternatively, polynucleotides may be synthesized or selected by use of the redundancy in the genetic code. Various codon substitutions may be introduced, e.g., by silent changes (thereby producing various restriction sites) or to optimize expression for a particular system. Mutations may be introduced to modify the properties of the polypeptide, perhaps to change ligand-binding affinities, interchain affinities, or the polypeptide degradation or turnover rate.

Portions of the polynucleotide sequence having at least about eight nucleotides, usually at least about 15 nucleotides, and usually fewer than about 1 kb, usually fewer than about 0.5 kb, from a polynucleotide sequence encoding a target gene are preferred as probes, although probes of other sizes may be used as desired.

Probes comprising synthetic oligonucleotides or other polynucleotides of the present invention may be derived from naturally occurring or recombinant single- or double-stranded polynucleotides, or be chemically synthesized. Probes may also be labeled by nick translation, Klenow fill reaction, or other methods that, in view of this specification, are known in the art.

Commonly, gene expression data is obtained by employing an array of probes that hybridize to several, and even thousands or more different transcripts. Such arrays are often classified as microarrays or macroarrays, and this classification depends on the size of each position on the array. Herein, the term “microarray” is used to refer to arrays wherein the probe dinsity is greater than about 100 different probes per cm³.

In one embodiment, the present invention also provides a method wherein nucleic acid probes are immobilized on or in a solid or semisolid support in an organized array. Oligonucleotides can be bound to a support by a variety of processes, including lithography, and where the support is solid, it is common in the art to refer to such an array as a “chip”, although this parlance is not intended to indicate that the support is silicon or has any useful conductive properties. For example a chip can hold more than 250,000 oligonucleotides (GeneChip, Affymetrix). These nucleic acid probes comprise a nucleotide sequence at least about 12 nucleotides in length, preferably at least about 15 nucleotides, more preferably at least about 25 nucleotides, and most preferably at least about 40 nucleotides, and up to all or nearly all of a sequence which is complementary to a portion of the coding sequence of one or more target genes, such as a gene selected from any of Tables 1, 2, and 3.

In one embodiment, the nucleic acid probes are spotted onto a substrate in a two-dimensional matrix or array. Samples of nucleic acids can be labeled and then hybridized to the probes. Double-stranded nucleic acids, comprising the labeled or tagged sample nucleic acids bound to probe nucleic acids, can be detected once the unbound portion of the sample is washed away.

The probe nucleic acids can be spotted on substrates including glass, nitrocellulose, etc. The probes can be bound to the substrate by either covalent bonds or by non-specific interactions, such as hydrophobic interactions. The sample nucleic acids can be labeled using radioactive labels, fluorophores, chromophores, etc. The sample nucleic acids may also be tagged with a tag that interacts with a separate label. For example, the sample nucleic acids may be generated so as to incorporate a specific and uniform tag nucleic acid sequence. Such tagged nucleic acids may be detected by contacting them with a labeled molecule that includes a tag-binding element, such as a complementary sequence. Such tagging systems provide amplification to the signal.

In other embodiments, the sample nucleic acid is not labeled. In this case, hybridization can be determined, e.g., by plasmon resonance, as described, e.g., in Thiel et al. (1997) Anal. Chem. 69:4948.

In one embodiment, a plurality (e.g., 2, 3, 4, 5 or more) of sets of sample nucleic acids are labeled and used in one hybridization reaction (“multiplex” analysis). For example, one set of nucleic acids may correspond to RNA from one cell and another set of nucleic acids may correspond to RNA from another cell. The plurality of sets of nucleic acids can be labeled with different labels, e.g., different fluorescent labels which have distinct emission spectra so that they can be distinguished. The sets can then be mixed and hybridized simultaneously to one microarray.

For example, the two different cells can be a diseased cell of a patient having a disease and a counterpart normal cell. Alternatively, the two different cells can be a diseased cell of a patient having a disease and a diseased cell of a patient suspected of having the disease. In another embodiment, one biological sample is exposed to a drug and another biological sample of the same type is not exposed to the drug. The cDNA derived from each of the two cell types are differently labeled so that they can be distinguished. In one embodiment, for example, cDNA from a diseased cell is synthesized using a fluorescein-labeled dNTP, and cDNA from a second cell, i.e., the normal cell, is synthesized using a rhodamine-labeled dNTP. When the two cDNAs are mixed and hybridized to the microarray, the relative intensity of signal from each cDNA set is determined for each site on the array, and any relative difference in abundance of a particular mRNA detected.

In the example described above, the cDNA from the diseased cell will fluoresce green when the fluorophore is stimulated and the cDNA from the cell of a subject suspected of having disease will fluoresce red. As a result, if the two cells are essentially the same, the particular mRNA will be equally prevalent in both cells and, upon reverse transcription, red-labeled and green-labeled cDNA will be equally prevalent. When hybridized to the microarray, the binding site(s) for that species of RNA will emit wavelengths characteristic of both fluorophores (and appear brown in combination). In contrast, if the two cells are different, the ratio of green to red fluorescence will be different.

The use of a two-color fluorescence labeling and detection scheme to define alterations in gene expression has been described, e.g., in Shena et al., 1995, Quantitative monitoring of gene expression patterns with a complementary DNA microarray, Science 270:467-470. An advantage of using cDNA labeled with two different fluorophores is that a direct and internally controlled comparison of the mRNA levels corresponding to each arrayed gene in two cell states can be made, and variations due to minor differences in experimental conditions (e.g, hybridization conditions) will not affect subsequent analyses.

Typically, the arrays used in the present invention will have a site density of greater than 100 different probes per cm². Preferably, the arrays will have a site density of greater than 500/cm², more preferably greater than about 1000/cm², and most preferably, greater than about 10,000/cm². Preferably, the arrays will have more than 100 different probes on a single substrate, more preferably greater than about 1000 different probes still more preferably, greater than about 10,000 different probes and most preferably, greater than 100,000 different probes on a single substrate.

In certain embodiments, the invention provides specialized probe sets and arrays comprising such probe sets. Specialized probe sets comprise probes designed to hybridize to transcripts and complements thereof of a limited number of genes known to be related to a biological state of interest. A specialized array comprises a specialized probe set affixed to a solid or semisolid support. Exemplary specialized probe sets comprise probes for the detection of two or more genes selected from Tables 1, 2, or 3, and often three, four, five, ten, twenty, thirty, forty or fifty or more such genes. Specialized probe sets may, for advantages of cost and convenience, not have a full complement of probes designed to detect over 90% of the known genes in the target organism(s). Instead, a specialized probe set may contain probes corresponding to fewer than 80% of the known genes, fewer than 60%, fewer than 50% or fewer than 25% of the known genes of the target organism(s). Specialized probe sets may comprise probes corresponding to fewer than 10,000 genes, fewer than 5,000 genes, fewer than 2000 genes, fewer than 1000 genes, fewer than 500 genes, fewer than 100 genes, fewer than 50 genes, fewer than 30 genes or fewer than 20 genes.

Microarrays can be prepared by methods known in the art, as described below, or they can be custom made by companies, e.g., Affymetrix (Santa Clara, Calif.).

Two types of microarrays are most commonly used. These two types are referred to as “synthesis” and “delivery.” In the synthesis type, a microarray is prepared in a step-wise fashion by the in situ synthesis of nucleic acids from nucleotides. With each round of synthesis, nucleotides are added to growing chains until the desired length is achieved. In the delivery type of microarray, preprepared nucleic acids are deposited onto known locations using a variety of delivery technologies. Numerous articles describe the different microarray technologies, e.g., Shena et al. (1998) Tibtech 16: 301; Duggan et al. (1999) Nat. Genet. 21:10; Bowtell et al. (1999) Nat. Genet. 21: 25.

“Delivery” microarrays can be prepared by mechanical microspotting. According to these methods, small quantities of nucleic acids are printed onto solid surfaces. Microspotted arrays prepared by many manufacturers contain as many as 10,000 groups of probes in an area of about 3.6 cm². Other “delivery” approaches include ink-jetting technologies, which utilize piezoelectric and other forms of propulsion to transfer nucleic acids from miniature nozzles to solid surfaces. Inkjet technologies are available through several centers including Incyte Pharmaceuticals (Palo Alto, Calif.) and Protogene (Palo Alto, Calif.). This technology may provide a density of 10,000 spots per cm². See also, Hughes et al. (2001) Nat. Biotechn. 19:342.

Arrays preferably include control and reference probes. Control probes are nucleic acids which serve to indicate that the hybridization was effective. For example, arrays for detection of human transcripts often contain sets of probes for several prokaryotic genes, e.g., bioB, bioC and bioD from biotin synthesis of E. coli and cre from P1 bacteriophage. Hybridization to these arrays is conducted in the presence of a mixture of these genes or portions thereof to confirm that the hybridization was effective. Control nucleic acids included with the target nucleic acids can also be mRNA synthesized from cDNA clones by in vitro transcription. Other control genes that are often included in arrays are polyA controls, such as dap, lys, phe, thr, and trp.

Reference probes allow the normalization of results from one experiment to another, and to compare multiple experiments on a quantitative level. Reference probes are typically chosen to correspond to genes that are expressed at a relatively constant level across different cell types and/or across different culture conditions. Exemplary reference nucleic acids include housekeeping genes of known expression levels, e.g., GAPDH, hexokinase and actin.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases.

Arrays may also contain probes that hybridize to more than one allele or one or more splice variant of a gene. For example the array can contain one probe that recognizes allele 1 and another probe that recognizes allele 2 of a particular gene.

Exemplary techniques for constructing arrays and methods of using these arrays are described in EP No. 0 799 897; PCT No. WO 97/29212; PCT No. WO 97/27317; EP No. 0 785 280; PCT No. WO 97/02357; U.S. Pat. No. 5,593,839; U.S. Pat. No. 5,578,832; EP No. 0 728 520; U.S. Pat. No. 5,599,695; EP No. 0 721 016; U.S. Pat. No. 5,556,752; PCT No. WO 95/22058; U.S. Pat. No. 5,631,734; U.S. Pat. No. 6,083,697; and U.S. Pat. No. 6,051,380.

When using commercially available microarrays, adequate hybridization conditions are provided by the manufacturer. When using non-commercial microarrays, adequate hybridization conditions can be determined based on the following hybridization guidelines, as well as on the hybridization conditions described in the numerous published articles on the use of microarrays.

Nucleic acid hybridization and wash conditions are optimally chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. When a certain degree of mismatching between probe and target is anticipated, the hybridization conditions may be relaxed.

The length of the probe and GC content will determine the Tm of the hybrid, and thus the hybridization conditions necessary for obtaining specific hybridization of the probe to the template nucleic acid. These factors are well known to a person of skill in the art, and can also be tested in assays. An extensive guide to the hybridization of nucleic acids is found in Tijssen (1993), “Laboratory Techniques in biochemistry and molecular biology-hybridization with nucleic acid probes.” Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Highly stringent conditions are selected to be equal to the Tm point for a particular probe. Sometimes the term “Td” is used to define the temperature at which at least half of the probe dissociates from a perfectly matched target nucleic acid. In any case, a variety of estimation techniques for estimating the Tm or Td are available, and generally described in Tijssen, supra. Typically, G-C base pairs in a duplex are estimated to contribute about 3° C. to the Tm, while A-T base pairs are estimated to contribute about 2° C., up to a theoretical maximum of about 80-100° C. However, more sophisticated models of Tm and Td are available and appropriate in which G-C stacking interactions, solvent effects, the desired assay temperature and the like are taken into account. For example, probes can be designed to have a dissociation temperature (Td) of approximately 60° C., using the formula: Td=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the annealing of the probe to the template DNA.

The stability difference between a perfectly matched duplex and a mismatched duplex, particularly if the mismatch is only a single base, can be quite small, corresponding to a difference in Tm between the two of as little as 0.5 degrees. See Tibanyenda, N. et al., Eur. J. Biochem. 139:19 (1984) and Ebel, S. et al., Biochem. 31:12083 (1992). More importantly, it is understood that as the length of the homology region increases, the effect of a single base mismatch on overall duplex stability decreases.

Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part I chapter 2 “Overview of principles of hybridization and the strategy of nucleic acid probe assays”, Elsevier, New York provide a basic guide to nucleic acid hybridization.

Background signal may be reduced by the use of a detergent (e.g, C-TAB) or a blocking reagent (e.g., sperm DNA, cot-1 DNA, etc.) during the hybridization to reduce non-specific binding. The use of blocking agents in hybridization is well known to those of skill in the art (see, e.g., Chapter 8 in Laboratory Techniques in Biochemistry and Molecular Biology, Vol. 24: Hybridization With Nucleic Acid Probes, P. Tijssen, ed. Elsevier, N.Y., (1993)). The method may or may not further comprise a non-bound label removal step prior to the detection step, depending on the particular label employed on the target nucleic acid. One means of removing the non-bound labeled target is to perform the well known technique of washing, where a variety of wash solutions and protocols for their use in removing non-bound label are known to those of skill in the art and may be used.

The above steps result in the production of hybridization patterns of labeled target nucleic acid on the array surface. The resultant hybridization patterns may be visualized or detected in a variety of ways, with the particular manner of detection being chosen based on the particular label of the target nucleic acid, where representative detection means include scintillation counting, autoradiography, fluorescence measurement, colorimetric measurement, light emission measurement, light scattering, and the like.

One method of detection includes an array scanner that is commercially available from Affymetrix (Santa Clara, Calif.), e.g., the 417™ Arrayer, the 418™ Array Scanner, or the Agilent GeneArray™ Scanner. This scanner is controlled from the system computer with a Windows® interface and easy-to-use software tools. The output is a 16-bit.tif file that can be directly imported into or directly read by a variety of software applications. Preferred scanning devices are described in, e.g., U.S. Pat. Nos. 5,143,854 and 5,424,186.

When fluorescently labeled probes are used, the fluorescence emissions at each site of a transcript array can be detected by scanning confocal laser microscopy. In one embodiment, a separate scan, using the appropriate excitation line, is carried out for each fluorophore used. Alternatively, a laser can be used that allows simultaneous specimen illumination at wavelengths specific to more than one fluorophore and emissions from more than one fluorophore can be analyzed simultaneously. Fluorescence laser scanning devices are described in Schena et al., 1996, Genome Res. 6:639-645 and in other references cited herein. Alternatively, the fiber-optic bundle described by Ferguson et al., 1996, Nature Biotech. 14:1681-1684, may be used to monitor mRNA abundance levels.

Following the data gathering operation, the data will typically be reported to a data analysis system. To facilitate data analysis, the data obtained by the reader from the device will typically be analyzed using a digital computer. Typically, the computer will be appropriately programmed for receipt and storage of the data from the device, as well as for analysis and reporting of the data gathered, e.g., subtraction of the background, deconvolution multi-color images, flagging or removing artifacts, verifying that controls have performed properly, normalizing the signals, interpreting fluorescence data to determine the amount of hybridized target, normalization of background and single base mismatch hybridizations, and the like. Various analysis methods that may be employed in such a data analysis system, or by a separate computer are described herein.

A desirable system for analyzing data is a general and flexible system for the visualization, manipulation, and analysis of gene expression data. Such a system preferably includes a graphical user interface for browsing and navigating through the expression data, allowing a user to selectively view and highlight the genes of interest. The system also preferably includes sort and search functions and is preferably available for general users with PC, Mac or Unix workstations. Also preferably included in the system are clustering algorithms that are qualitatively more efficient than existing ones. The accuracy of such algorithms is preferably hierarchically adjustable so that the level of detail of clustering can be systematically refined as desired.

While the above discussion focuses on the use of arrays for the collection of gene expression data, such data may also be obtained through a variety of other methods, that, in view of this specification, are known to one of skill in the art.

A method for high throughput analysis of gene expression is the serial analysis of gene expression (SAGE) technique, first described in Velculescu et al. (1995) Science 270, 484-487. Among the advantages of SAGE is that it has the potential to provide detection of all genes expressed in a given cell type, whether previously identified as genes or not, provides quantitative information about the relative expression of such genes, permits ready comparison of gene expression of genes in two cells, and yields sequence information that can be used to identify the detected genes. Thus far, SAGE methodology has proved itself to reliably detect expression of regulated and nonregulated genes in a variety of cell types (Velculescu et al. (1997) Cell 88, 243-251; Zhang et al. (1997) Science 276, 1268-1272 and Velculescu et al. (1999) Nat. Genet. 23, 387-388.

For example, gene expression data may be gathered by RT-PCR. mRNA obtained from a sample is reverse transcribed into a first cDNA strand and subjected to PCR. House keeping genes, or other genes whose expression is fairly constant can be used as internal controls and controls across experiments. Following the PCR reaction, the amplified products can be separated by electrophoresis and detected. Taqman™ fluorescent probes, or other detectable probes that become detectable in the presence of amplified product may also be used to quantitate PCR products. By using quantitative PCR, the level of amplified product will correlate with the level of RNA that was present in the sample. The amplified samples can also be separated on a agarose or polyacrylamide gel, transferred onto a filter, and the filter hybridized with a probe specific for the gene of interest. Numerous samples can be analyzed simultaneously by conducting parallel PCR amplification, e.g., by multiplex PCR.

Transcript levels may also be determined by dotblot analysis and related methods (see, e.g., G. A. Beltz et al., in Methods in Enzymology, Vol. 100, Part B, R. Wu, L. Grossmam, K. Moldave, Eds., Academic Press, New York, Chapter 19, pp. 266-308, 1985). In one embodiment, a specified amount of RNA extracted from cells is blotted (i.e., non-covalently bound) onto a filter, and the filter is hybridized with a probe of the gene of interest. Numerous RNA samples can be analyzed simultaneously, since a blot can comprise multiple spots of RNA. Hybridization is detected using a method that depends on the type of label of the probe. In another dotblot method, one or more probes of one or more genes characteristic of disease D are attached to a membrane, and the membrane is incubated with labeled nucleic acids obtained from and optionally derived from RNA of a cell or tissue of a subject. Such a dotblot is essentially an array comprising fewer probes than a microarray.

Another format, the so-called “sandwich” hybridization, involves covalently attaching oligonucleotide probes to a solid support and using them to capture and detect multiple nucleic acid targets (see, e.g., M. Ranki et al., Gene, 21, pp. 77-85, 1983; A. M. Palva, T. M. Ranki, and H. E. Soderlund, in UK Patent Application GB 2156074A, Oct. 2, 1985; T. M. Ranki and H. E. Soderlund in U.S. Pat. No. 4,563,419, Jan. 7, 1986; A. D. B. Malcolm and J. A. Langdale, in PCT WO 86/03782, Jul. 3, 1986; Y. Stabinsky, in U.S. Pat. No. 4,751,177, Jan. 14, 1988; T. H. Adams et al., in PCT WO 90/01564, Feb. 22, 1990; R. B. Wallace et al. 6 Nucleic Acid Res. 11, p. 3543, 1979; and B. J. Connor et al., 80 Proc. Natl. Acad. Sci. USA pp. 278-282, 1983). Multiplex versions of these formats are called “reverse dot blots.”

mRNA levels can also be determined by Northern blots. Specific amounts of RNA are separated by gel electrophoresis and transferred onto a filter which is then hybridized with a probe corresponding to the gene of interest.

The level of expression of one or more genes in a cell may be determined by in situ hybridization. In one embodiment, a tissue sample is obtained from a subject, the tissue sample is sliced, and in situ hybridization is performed according to methods known in the art, to determine the level of expression of the genes of interest. Gene expression may also be monitored by use of a reporter gene (eg. lacZ, cat, GUS, gfp, etc.) linked to the relevant promoter.

Techniques for producing and probing nucleic acids are further described, for example, in Sambrook et al., “Molecular Cloning: A Laboratory Manual” (New York, Cold Spring Harbor Laboratory, 1989).

4. Protein Expression Data

In general, protein expression data may be gathered in any way that, in view of this specification, is available to one of skill in the art. Although many analytical methods provided herein are powerful tools for the analysis of protein data obtained by highly parallel data collection systems, many such methods are equally useful for the analysis of data gathered by more traditional methods.

Immunoassays are commonly used to quantitate the levels of proteins in samples, and many other immunoassay techniques are known in the art. The invention is not limited to a particular assay procedure, and therefore is intended to include both homogeneous and heterogeneous procedures. Exemplary immunoassays which can be conducted according to the invention include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

In yet another embodiment, the invention contemplates using a panel of antibodies which are generated against the marker polypeptides of this invention, which polypeptides are encoded in Table 1. Such a panel of antibodies may be used as a reliable diagnostic probe for hyperproliferative disorders.

Where tissue samples are employed, immunohistochemical staining may be used to determine the number of cells having the marker polypeptide phenotype. For such staining, a multiblock of tissue is taken from the biopsy or other tissue sample and subjected to proteolytic hydrolysis, employing such agents as protease K or pepsin. In certain embodiments, it may be desirable to isolate a nuclear fraction from the sample cells and detect the level of the marker polypeptide in the nuclear fraction.

The tissue samples are fixed by treatment with a reagent such as formalin, glutaraldehyde, methanol, or the like. The samples are then incubated with an antibody, preferably a monoclonal antibody, with binding specificity for the marker polypeptides. This antibody may be conjugated to a label for subsequent detection of binding. Samples are incubated for a time sufficient for formation of the immuno-complexes. Binding of the antibody is then detected by virtue of a label conjugated to this antibody. Where the antibody is unlabeled, a second labeled antibody may be employed, e.g., which is specific for the isotype of the anti-marker polypeptide antibody. Examples of labels which may be employed include radionuclides, fluorescers, chemiluminescers, enzymes and the like.

Where enzymes are employed, the substrate for the enzyme may be added to the samples to provide a colored or fluorescent product. Examples of suitable enzymes for use in conjugates include horseradish peroxidase, alkaline phosphatase, malate dehydrogenase and the like. Where not commercially available, such antibody:enzyme conjugates are readily produced by techniques known to those skilled in the art.

Protein levels may be detected by a variety of gel based methods. For example, proteins may be resolved by gel electrophoresis, preferably two-dimensional electrophoresis comprising a first dimension based on pI and a second dimension of denaturing PAGE. Proteins resolved by electrophoresis may be labeled beforehand by metabolic labeling, such as with radioactive sulfur, carbon, nitrogen and/or hydrogen labels. If phosphorylation levels are of interest, proteins may be metabolically labeled with a phosphorus isotope. Radioactively labeled proteins may be detected by autoradiography, or by use of a commercially available system such as the PhosphorImager™ available from Molecular Dynamics (Amersham). Proteins may also be detected with a variety of stains, including but not limited to, Coomassie Blue, Ponceau S, silver staining, amido black, SYPRO dyes, etc. Proteins may also be excised from gels and subjected to mass spectroscopic analysis for identification. Gel electrophoresis may be preceded by a variety of fractionation steps to generate various subfractionated pools of proteins. Such fractionation steps may include, but are not limited to, ammonium sulfate precipitation, ion exchange chromatography, reverse phase chromatography, hydrophobic interaction chromatography, hydroxylapatite chromatography and any of a variety of affinity chromatography methods.

Proteins expression levels may also be measured through the use of a protein array. For example, one type of protein array comprises an array of antibodies of known specificity to particular proteins. Antibodies may be affixed to a support by, for example the natural interaction of antibodies with supports such as PVDF and nitrocellulose, or, as another example, by interaction with a support that is covalently associated with protein A (see for example U.S. Pat. No. 6,197,599), which binds tightly to the constant region of IgG antibodies. Antibodies may be spotted onto supports using technology similar to that described above for spotting nucleic acid probes onto supports. In another example, an array is prepared by coating a surface with a self-assembling monolayer that generates a matrix of positions where protein capture agents can be bound, and protein capture agents range from antibodies (and variants thereof) to aptamers, phage coat proteins, combinatorially derived RNAs, etc. (U.S. Pat. No. 6,329,209). Proteins bound to such arrays may be detected by a variety of methods known in the art. For example, proteins may be metabolically labeled in the sample with, for example, a radioactive label. Detection may then be accomplished using devices as described above. Proteins may also be labeled after being isolated from the sample, with, for example, a cross-linkable fluorescent agent. In one example, proteins are desorbed from the array by laser and subjected to mass spectroscopy for identification (U.S. Pat. No. 6,225,047). In another variation, the array may be designed for detection by surface plasmon resonance. In this case, binding is detected b changes in the surface plasmon resonance of the support (see, for example, Brockman and Fernandez, American Laboratory (June, 2001) p.37).

5. Methods Based on Measures of Variability

In certain aspects, the invention relates to methods of analyzing gene and protein expression data comprising examining a measure of variability. The preceding sections describe a variety of approaches that may be employed to collect gene and protein expression data. The manner by which such data is collected, stored and accessed is insignificant with respect to the novel methods for analysis of such data that are described herein. It is understood that one of skill in the art may employ any method for gathering gene and protein data and that such methods are likely to change rapidly in the future, and further that the methods for analysis described herein will be useful in the analysis of all such data.

In some embodiments, the invention relates to identifying a gene or protein that is relevant to the cellular state of a particular class of sample, and in some embodiments, the gene or protein may be causative or mechanistically involved in a difference in cellular states between two or more classes of sample.

In general, gene or protein expression data that is suitable for identifying a gene or protein that is relevant to the cellular state of a particular class of sample is accessed. Such data will typically represent gene or protein expression levels of one or more genes (G genes) in one or more samples (S samples) that may be classified into classes (C classes) representing different cellular states. Classes representing different cellular states may be defined by any relevant information about the sample. Depending on the cellular states of interest, the same samples may be reclassified for different analyses. For example, a set of samples representing bacteria grown in low and high phosphate conditions and then sampled in exponential phase and stationary phase may be sorted in different ways. If desired, the samples may be grouped into classes representing cellular states of cells grown in low and high phosphate conditions. Alternatively, the samples may be grouped into classed representing cellular states of cells in exponential or stationary phases. Additionally, samples may be classified into four classes for analysis: low phosphate, exponential phase; high phosphate, exponential phase; low phosphate, stationary phase; and high phosphate, stationary phase. Accordingly, as illustrated by this example, classification is not rigid and may be determined by one of skill in the art depending on the question of interest.

In certain embodiments, genes or proteins which are relevant to the differences between classes that represent cellular states are defined as those that have discriminatory power between the classes. Genes or proteins that have discriminatory power between distinct classes of samples may be selected based on the relative magnitudes of between- and within-group measures of variability. Thus, genes whose expression distribution has high between-group variability (the groups are well-separated) and small within-group variability (the samples inside each group are relatively similar) are deemed to be discriminatory for the sample classes. The between-group variability (Bᵢ) of the expression of a certain gene i is proportional to the sum of the differences between group means of expression levels. The within-group variability of the expression of gene i (Wᵢ) is the sum of group variability of the expression levels of the gene in a single class. Measures of variability include, but are not limited to, variance, standard deviation, range and kurtosis, as well as simple transformations of each of the foregoing. In certain embodiments, a comparison of “within group” and “between group” variability is accomplished by testing the null hypothesis that these two forms of variability are not different. When the null hypothesis is disproved with a confidence of at least 0.8, alternatively at least 0.85 or 0.9, preferably 0.95 and possibly even 0.99, then it may be concluded that the gene is related to the differences between the cellular states. The null hypothesis may be tested by using a variety of comparative statistical tests that, in view of this specification, may be selected by one of skill in the art. Such a test may involve, for example, a t-test, an F-test, or a gamma test.

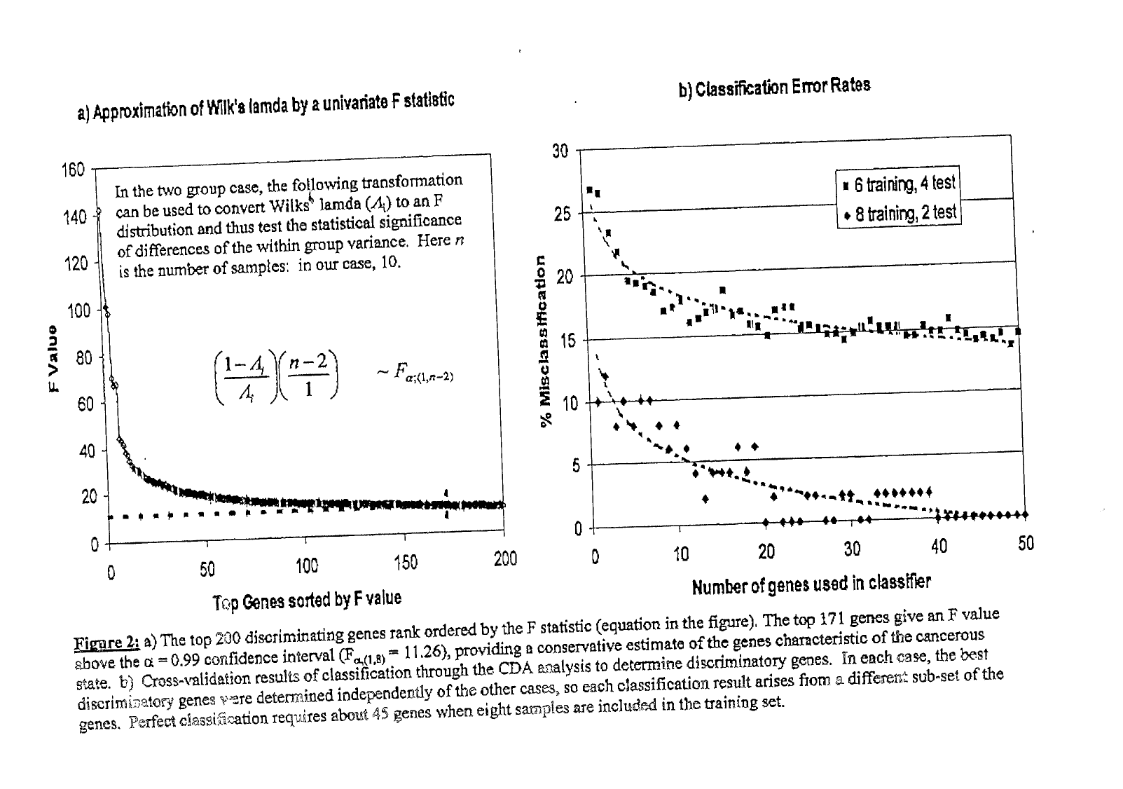

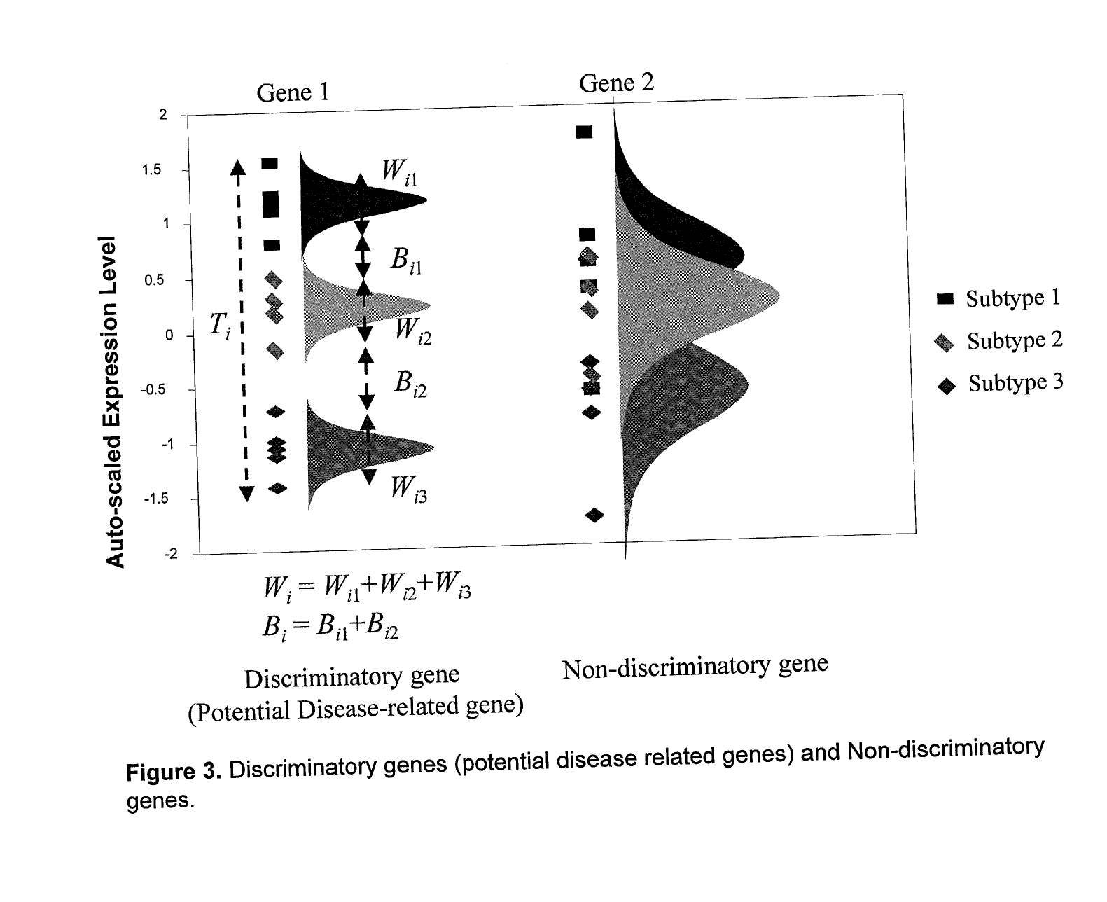

In certain embodiments, as shown in FIG. 2, the ratio of the “within group variance” to the “total variance” (also known as the “Wilks' lambda score”) can be used as a metric of each gene's class differentiating potential. Wilks' lambda scores may be transformed into a univariate F statistic that allows one to identify discriminatory genes with a specified statistical significance. By this approach, genes are identified whose between-group-variance is significantly larger, under the level of significance (a=0.1, 0.05, 0.03, or 0.01 or higher), than the within-group-variance.

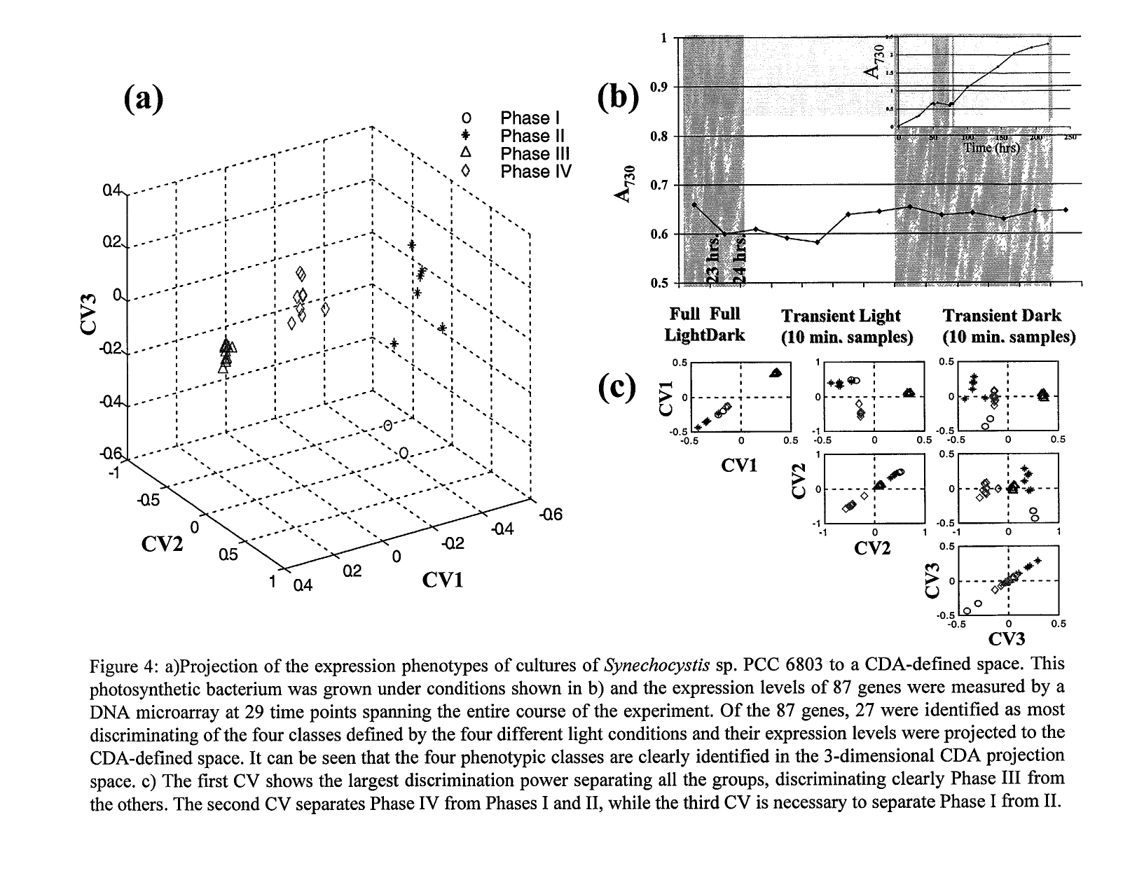

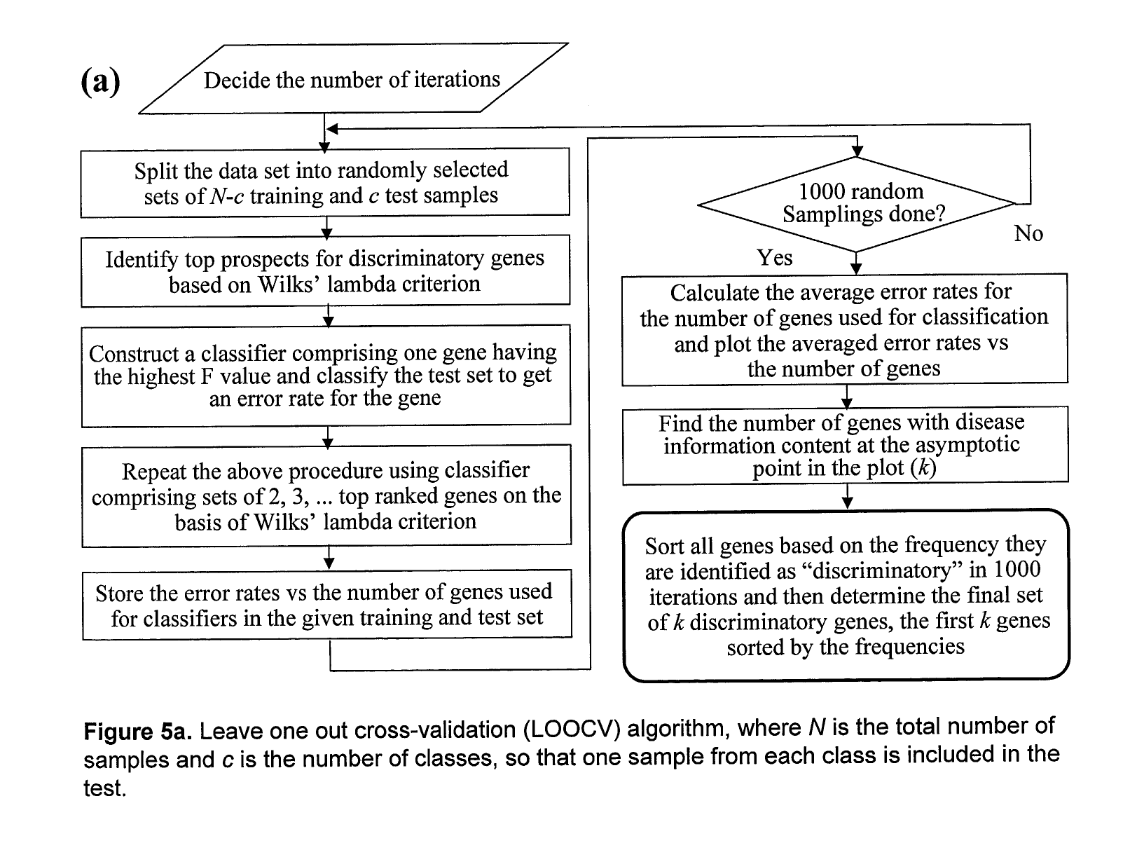

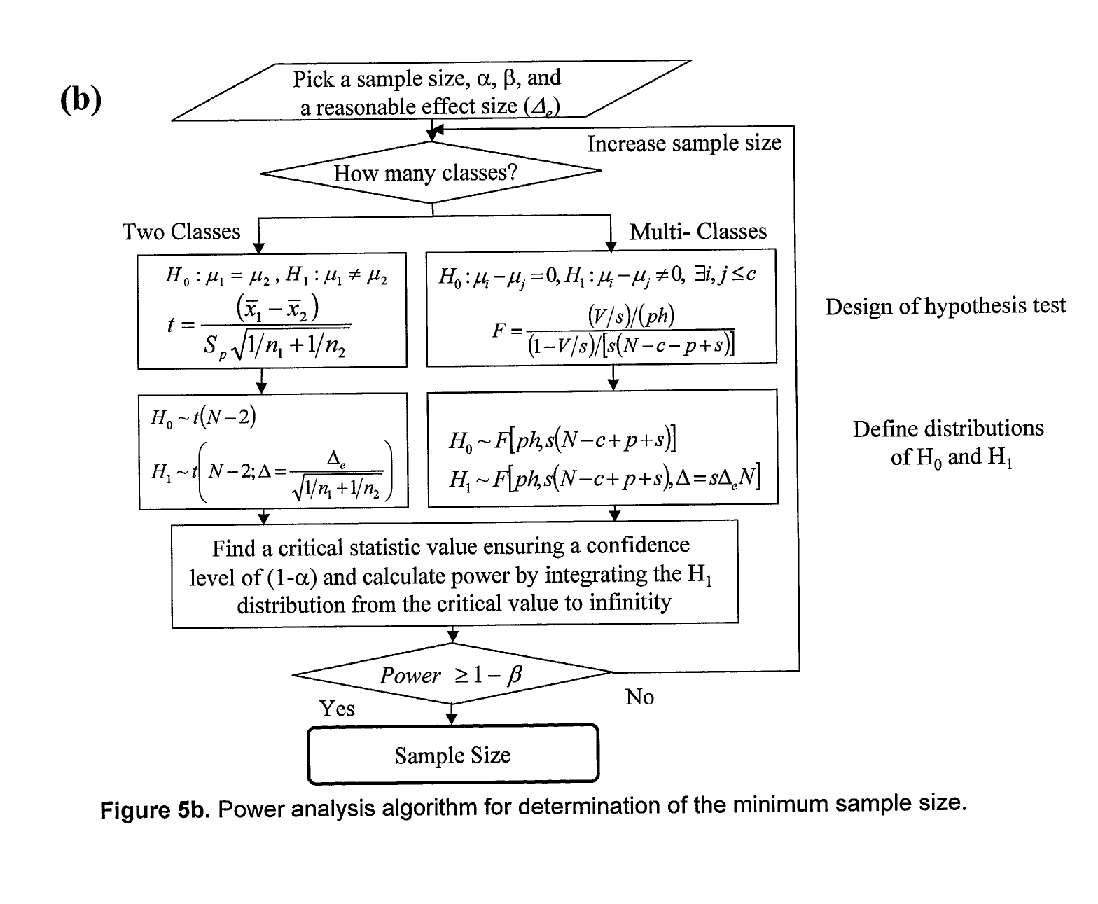

A gene or protein that is identified in this manner may be reflective of the difference in cellular state or may in some way cause or maintain the differences in cellular state. In either case, such genes or proteins are useful for a range of purposes including but not limited to diagnostic tests to distinguish samples of unknown classification and manipulation of cellular state by manipulation of the expression or function of the identified gene or protein.

Where the number of genes, G, is large, it may be advantageous to use matrix methods for the analysis of data. For a data matrix D comprising G columns of gene expression data measured in a total of S number of samples (rows) that can be a priori classified in C classes (or groups), an analytical method termed FDA defines a linear projection to a lower dimensional space such that the mean differences among the C classes are maximized. The FDA method is described schematically in FIG. 4 depicting a 2-D projection (along discriminant axes) of the expression data of three genes such that the separation of the three sample classes is maximized. Each coordinate in the FDA space is defined as a linear combination of the actual gene expression measurements and is obtained by spectral decomposition of the “between group variance”. The coefficient multiplying each gene expression provides a measure of that gene's importance in defining the projection. The variables defining the new projection space are termed Fisher variables (FVs) and the coefficients multiplying the gene expression data discriminant loadings (also termed discriminant axes). It has been reported for similar problems that factor rotation of the discriminant loadings under Varimax criterion can be performed to extract some physical significance attributed to the loadings. There is a maximum of (C-1) discriminant loadings for C classes, so the number of classes dictates the number of dimensions that may be considered. If some discriminant loadings do not provide significant discrimination when tested by Bartlett's V statistics, they can be eliminated from discriminant analysis, allowing one to focus on important discriminant loadings.

Fisher Discriminant Analysis (FDA) is a classification method that operates by determining a set of dimensions where the separation between the given classes is maximized. The dimensions generated by FDA are known as the Discriminant Functions (DFs), and they are linear combinations of the primary variables, or dimensions. In the case of gene or protein expression data, the number of dimensions in the original data space is the number of genes, G, whose expression was monitored. Each DF is uncorrelated with the others, an aspect that increases the interpretability of the visualization.

A. Generation of Discriminant Axes/Linear Composites

In certain embodiments, matrix data is transformed so as to reveal sets of gene or protein expression levels that are useful in distinguishing different classes of sample. For example, FDA defines a projection from the original to a reduced space that maximizes the ratio of the variance-between-groups to the variance-within-groups. This is mathematically equivalent to maximizing the mean separation between the various groups or classes in the reduced dimensional space. If there are C classes in the data, the within-group-variance W and the between-group-variance B are respectively defined as:

$$W = {\sum }_{k = 1}^{c} {( {X}_{k} - 1 {\overline{x}}_{k} )}^{T} ( {X}_{k} - 1 {\overline{x}}_{k} ) \qquad ( 1 )$$

B=T−W=(X−1\overline{x})^{T}(X−1\overline{x})−W (2)

where T is the total variation. Xₖ and X are data matrices for samples in class k and the entire expression set, respectively. These matrices are organized such that X(i,j) is the expression of gene j in sample i. \overline{x}ₖ is the group mean (1×g) for class k, while \overline{x} is the mean for all the data. It can be proved that the separation between pre-defined groups in a reduced dimensional space is maximized when the space is defined by the eigenvectors of the matrix W⁻¹B (Dillon & Goldstein, 1984). Mathematically, the eigenvalue decomposition of the matrix is given by:

W⁻¹BL=LΛ (3)

The eigenvector matrix (L) defines the dimensions of the reduced space. Each column of L defines an axis or Discriminant Function (DF) of the FDA space. The diagonal entries of the eigenvalue matrix (Λ) represent the discriminant powers of each corresponding DF. The entries in L contain the discriminant weight for each gene. The discriminant weight determines the contribution of each gene in defining the DF. Finally, the projections of the individual samples onto each DF, or the discriminant score, is calculated by:

$${y}_{j} = x {L}_{j} = {\sum }_{i = 1}^{g} {x}_{i} {L}_{ij} \qquad ( 4 )$$

where yⱼ is the discriminant score of the actual sample x on the jth DF. The individual discriminant scores for a sample on each DF can be combined in a vector y, whose dimensionality is the number of dimensions in the FDA space.

In order to get robust classification from an eigenvalue decomposition, the variance-covariance structure is preferably similar in all the various classes. In cases where this assumption is not valid, we have found that the singular value decomposition of W⁻¹B produces better discriminant axes than the eigenvalue decomposition of W⁻¹B, and thus the axes more effectively capture the between-group variance. For those cases, the singular value decomposition was applied to find the axes and to calculate the discriminant scores:

W⁻¹B=UΛL^{T} (5)

where U is the left singular vector, L is the matrix of discriminant axes, or the DFs, and Λ is the matrix of singular values representing the discriminant powers along the corresponding axes. The calculation of the discriminant scores remains the same as before.

B. Contributions of Individual Genes (Predictor Variables) to Discrimination Among Classes

In certain embodiments, when the discrminant axes have been defined, it may be useful to determine the contribution of one or more genes to the discriminating function. For example, once the discriminating FDA projection is obtained, the discriminant weights are examined to determine the importance of each gene in the resulting classification. This approach is particularly useful for data sets where the variables (genes) are not strongly correlated with each other. In cases where the genes (or predictor variables) are significantly correlated, contributions of predictor variables to discrimination among classes may preferably be determined using discriminant loadings (L*), rather than using the discriminant weights (L):

L*=RD^{½}L (6)

where R is the correlation matrix of the data matrix and D^{½} is the diagonal matrix of standard deviations for predictor variables (genes) (Dillon and Goldstein 1984). The above equation calculates the correlation of a gene to a DF. This calculation is not impeded by the inter-correlation among genes. Hence, if two strongly correlated genes are both important for defining a DF, they will have similar loadings.

Each individual gene that is identified as contributing to the discrimination between classes may be useful for various purposes described herein. In addition, the collective group of genes and their expression levels define a pattern of gene expression that is useful for classifying samples of unknown classification. Patterns may include all of the relevant genes or the patterns may be subcombination of the contributing genes. Exemplary methods of selecting subcombination patterns are described below, but it is understood that a variety of methods are, in view of this specification available for selecting such patterns are known in the art.

C. Pre-Selection of Discriminating Genes

When a large number of variables (here, gene or protein expression levels) are employed, the risk of obtaining a poor FDA classification increases due to the increased likelihood of noisy variables. Therefore, it may, in some embodiments, be preferable to execute some form of gene selection methodology prior to classification, to screen out noisy and non-discriminating genes. In one embodiment, Wilks' lambda may be employed, defined as the ratio of the determinant of the between-group variance matrix W to the determinant of the total variance matrix T for each gene, to obtain an initial set of discriminatory genes (Dillon and Goldstein 1984). Wilks' lambda can be transformed into an F-distribution, which allows the selection of discriminatory genes with an appropriate confidence level. A preferred confidence level is 0.8, while particularly preferred confidence levels are 0.85, 0.9, 0.95 and most preferably 0.99.

In certain embodiments, the set of genes may be further refined by retaining only those genes that yield low misclassification rates in a leave-one-out-cross-validation procedure. For example, in the construction of an FDA classifier, one sample from each class may be removed from the analysis, and the classifier built on the remaining samples using the given set of genes. The classifier is then used to predict the class of the withheld samples. This procedure is repeated for all the samples for the given set of genes, and the final cumulative error is recorded. Then, the gene with the lowest value of Wilks' lambda is removed, and the cross-validation procedure repeated to obtain a new cumulative error. In certain embodiments, the set of genes that provides a minimum cumulative error may be chosen for use in future classifications. In other embodiments, a set of genes may be selected that provides a misclassification rate of a pre-set tolerable amount. In certain embodiments, the misclassification rate is less than 40%, optionally less than 30%, less than 20%, less than 15%, less than 10% or less than 5%.

For the purposes of clarity and to illustrate additional embodiments, the following description of certain refinement methodologies is provided. In certain embodiments, a stronger classification criterion may be obtained by using an error classification rate. In these methods, a subset of the available samples (the training set) is used to identify the discriminating genes as well as to define a sample classification model. The classification model is subsequently tested against the samples that were not included in the training set (the test set) and the misclassification rate is calculated for all possible membership configurations of the training and test sets. This procedures is initiated with a classifier that is preferably based on a single (most discriminating) gene and is repeated as more genes (in order of discriminating power based on, for exmaple, their F value) are added to the classifier. The misclassification rate would be expected to decrease as more and more genes are added to the classifier, making it more robust. This is exactly what is observed with the expression data of oral epithelium cancer, as shown in FIG. 4b (see also Example 2 below). Clearly, 40-45 genes are sufficient to accurately predict the class of the samples in the test set and, as such, they are deemed most discriminatory of the oral epithelium cancerous state.

The misclassification rate is a function of both the sample population size and the number of genes considered. Even with only three samples describing each of the two states (that is, reserving 2 of the 5 samples to test the classifier developed using the other 3), correct classification is achieved over 85% of the time if a sufficient number of genes are considered. Four samples from each group (leave one out case) were sufficient to achieve perfect classification for all permutations of the training and testing sets when at least 45 genes are considered. These results show that accurate classification can be achieved even with only a few samples if a sufficient number of genes are included in the classifier. Furthermore, FIG. 4b shows that consideration of one or two genes as “markers” for disease is an insufficient measure of physiological state. The procedures of cross-validation is discussed further in FIG. 4b.

D. An Exemplary Description of Analysis of Gene or Protein Expression Data

Another exemplary embodiment for the analysis of gene or protein expression data is provided below. The between-group variance (Bᵢ) of the expression of a certain gene (or protein, although the term “gene” will be used throughout this exemplary embodiment) i is proportional to the sum of the differences between group means of expression levels. The within-group variance of the expression of gene i (Wᵢ) is the sum of group variances of the expression levels of the gene in a single class. With the total variance of expression levels of gene i, Tᵢ=(Xᵢ−1\overline{x}ᵢ)^{T}(xᵢ−1\overline{x}₁), the within- and the between-group variances are defined respectively as follows.

$${W}_{i} = {\sum }_{j = 1}^{c} {W}_{i}^{j} = {\sum }_{j = 1}^{c} {( {x}_{i}^{j} - 1 {\overline{x}}_{i}^{j} )}^{T} ( {x}_{i}^{j} - 1 {\overline{x}}_{i}^{j} ) \qquad ( 7 )$$

Bᵢ=Tᵢ−Wᵢ (8)

The vector, xᵢ (Nx1), contains the expression level of gene i in N samples and \overline{x}ᵢ is the mean expression of gene i in all N samples. The superscript j represents class j among the c classes. For the two genes shown schematically in FIG. 1, gene 1 has a large between-group variance and a small within-group variance while gene 2 has a small between-group variance (overlapping distributions across the classes) and a large within-group variance. For gene 1, the large ratio of the between-group variance to within-group variance indicates a gene with a discriminatory expression pattern.

The above procedure is implemented through a statistical test based on Wilks' lambda (Λᵢ) that allows one to establish a formal boundary between discriminatory genes and non-discriminatory genes:

$${Λ}_{i} = \frac{{W}_{i}}{{T}_{i}} \qquad ( 9 )$$

In order to compare the Wilks' lambda (Λᵢ) score to a distribution with known parameters, it is transformed to the F distribution as follows (Dillon & Goldstein, 1984; SAS 1989):

$${F}_{i} = \frac{( 1 - {Λ}_{i} )}{{Λ}_{i}} \frac{( N - c )}{( c - 1 )} \sim {F}_{a ( c - 1 , N - c )} \qquad ( 10 )$$

where N is the total number of samples and c is the number of classes. In this form, discriminatory genes are selected by applying a statistical cutoff determined from the F distribution using some level of significance (in this case α=0.01). Note that a high F value signifies a more discriminatory gene relative to one with a low F value. This is dependent on the level of significance (false positive errors) that is desired from the F test. If the level of significance is fixed, the number of classes and samples will determine the threshold. Thus, a small significance level leads to a stringent test (low false positive, but relatively high false negative), because false positive are inversely related to false negative in a given number of samples. Values of F are functions of the degrees of freedom, so a “low” value of F in one case =may be “high” in another case.

Fisher Discriminant Analysis (FDA) (Johnson & Wichern, 1992; Dillon & Goldstein, 1984; Xiong et al., 2000) is a linear method of dimensionality reduction from the expression space comprising all selected discriminatory genes to just a few dimensions where the separation of sample classes is maximized. FDA (Alter, 2000; Holter et al., 2000) in the linear reduction of data (Johnson & Wichern, 1992). An important difference between FDA and a technique termed PCA is that the discriminant axes of the FDA space are selected such as to maximize class separation in the reduced FDA space, instead of variability as in the case of PCA. The discriminant axes of FDA, termed as discriminant loadings (L), maximizing the separation of sample classes in their projection space can be shown to be equivalent to the eigenvectors of W⁻¹B, the ratio of between-group variance (B) to within-group variance (W). Since these directions capture maximal between-group variance and minimal within-group variance, the sample classes are maximally separated in this projection space. The eigenvalues are calculated as follows:

W⁻¹BL=LΛ (11)

$$where B = T - W , W = {\sum }_{j = 1}^{c} {( {X}_{j} - 1 {\overline{x}}_{j}^{T} )}^{T} ( {X}_{j} - 1 {\overline{x}}_{j}^{T} ) , \\ and$$

$$T = {( X - 1 {\overline{x}}^{T} )}^{T} ( X - 1 {\overline{x}}^{T} ) .$$

The eigenvalues (Λ) indicate the discrimination power for the corresponding discriminant axes. For expression data sets having a lager number of genes than samples, the number of discriminant axes in an FDA projection is one fewer than the number of classes considered. Discriminant loadings, which are measures of how each gene's expression impacts the projected value, allow us to understand what genes are important for discrimination of sample classes and how they act together to give a clear separation in the discrimination space.

A FDA classifier can then be developed in the projection space. A new sample is projected into the FDA space using the discriminant loadings (L). Then, a classification rule can be built in the FDA space such that the new sample will be assigned to the predefined class whose mean is closest to the projection of the new sample (Johnson & Wichern, 1992): a new sample (x) will be allocated to class j if

∥ŷ−\overline{y}ⱼ∥²=∥(\hat{x}−\overline{x}ⱼ)L∥²≦∥(\hat{x}−\overline{x}ₖ)L∥² for all k≠j

where ŷ is a projection of the new sample into the discriminant loadings (L).

In certain embodiments, and as described above, it may be desirable to narrow the list of genes to be incorporated into a discriminatory pattern. The list may be reduced on the basis of the misclassification (error) rates based on a modified version of leave one out cross-validation (LOOCV). The first step in this exemplary iterative procedure includes randomly dividing the data set being considered into c test samples (i.e. one test sample for each class) and N-c training samples. The training samples are used to generate an initial set of discriminatory genes using, for example, Wilks' lambda criterion. Using the gene with highest F value, a FDA classifier is constructed and the error rate calculated for the c test samples (see next section). A second classifier is then constructed using the top two discriminating genes, which is again applied to the test samples. The number of genes included in the classifier is thus sequentially increased to form more complex classifiers until all selected genes have been included. At each step, the number of misclassified samples is determined for calculation of the misclassification error rate (see next paragraph). A new division of the samples into training and test sets is then considered, and the procedure is repeated.

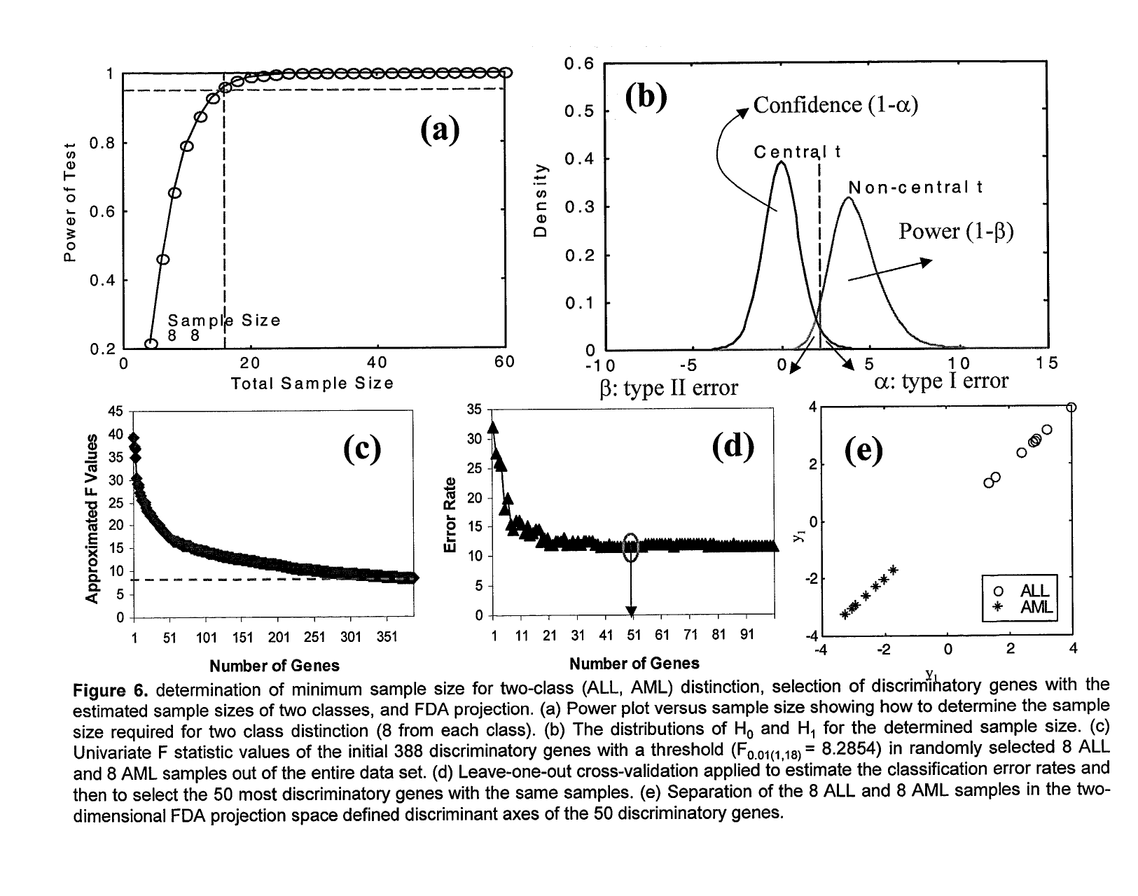

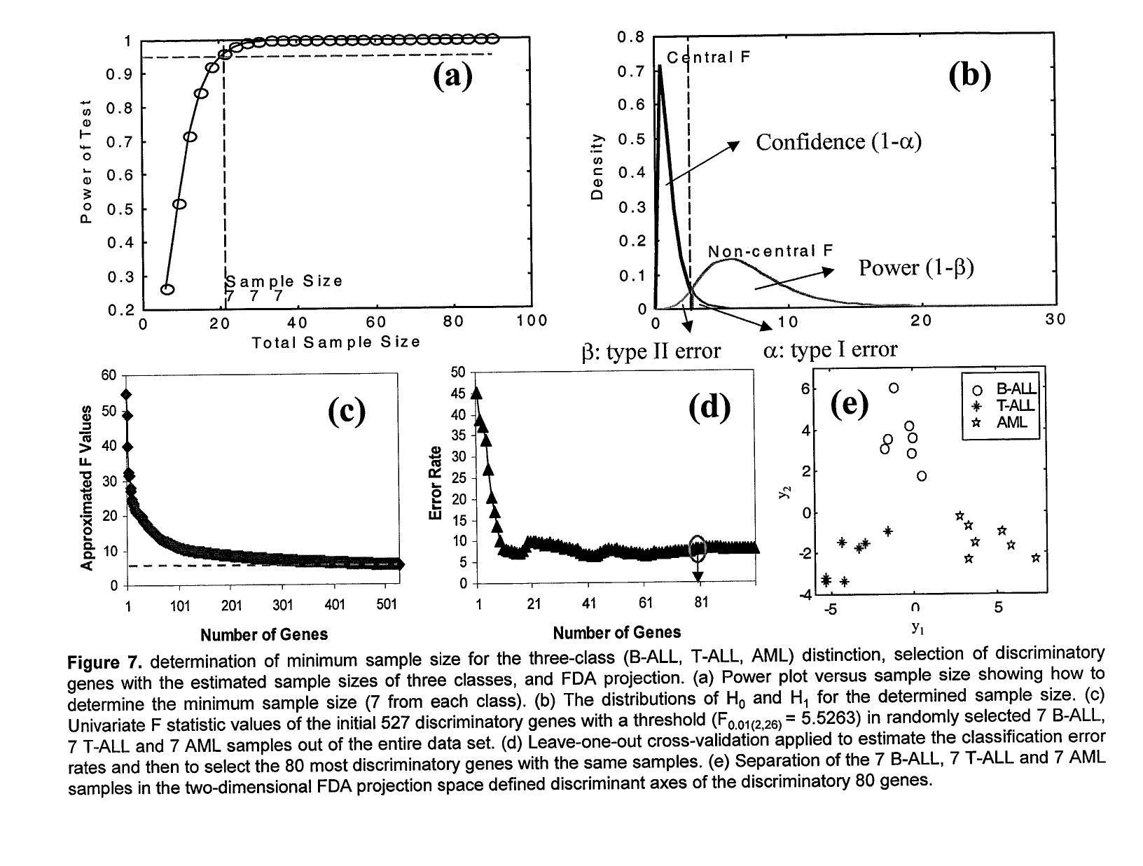

For the estimation of error rates, the LOOCV procedure is repeated at least 100 times, and preferably at least 1000 times, each time using a different, randomly selected set of N-c training and c test samples in the data set being considered. If we denote by mₚ the number of misclassified samples in the cross-validations for a given number of discriminatory genes (p) used in the classifiers, the estimated error rate is given by e(p)=mₚ/(c×1000). Then, the error rates from the cross-validation iterations can be computed as function of the number of discriminatory genes considered. Of all discriminatory genes identified by Wilks' lambda, those yielding a misclassification rate below a predetermined threshold (or at the asymptote of the graph) are preferably retained (see FIG. 6a and FIGS. 7d and 8d), although different subcombinations may be constructed.

E. Further Explanation of FDA

In general, although FDA comprises a spectral (or eigenvalue) decomposition method it is quite different from PCA. FDA seeks new canonical projection variables where the “between group variance” scaled by the “within group variance” is maximized. PCA instead maximizes the covariance matrix. Hence, the new canonical variables define directions along which the projected values exhibit the largest separation of points from different classes (groups). The canonical directions are obtained by multiplying (i.e. projecting) the original expression data matrix by a matrix V that is obtained by solving:

$${Max}_{v} \frac{{v}^{T} {W}^{- 1} B v}{{v}^{T} v}$$

where B is the “between group variance” (a measure of the interclass empty space) defined by B=T−W, with

$$\begin{matrix}T = {\sum }_{k = 1}^{c} ( X - \overline{x} {1}^{T} ) {( X - \overline{{x1}^{T}} )}^{T} & being the total variance and \\ T = {\sum }_{k = 1}^{c} ( {X}_{k} - \overline{{x}_{k}} {1}^{T} ) {( {X}_{k} - \overline{{x}_{k} {1}^{T}} )}^{T} & \begin{matrix}being the “ within \\ group variance ”\end{matrix}\end{matrix}$$

The solution to this maximization problem is obtained by eigenvalue decomposition (ED), yielding the above matrix V.

W⁻¹B=V ΛV^{T}

If the objective criterion is changed into “between group kurtosis”, (W¹B)^{T}W¹B, instead of W¹B, the solution of the maximization formulation will be singular value decomposition (SVD).

W⁻¹B=U ΛV^{T}

Sometimes, SVD presents a better separation than ED, when there are directions which have multiple distribution modes where each mode is associated with a distinct group.

Although FDA tries its best to separate groups from one another, if there are subgroups in a particular physiological group, FDA will produce the separated subgroups for that physiological group. In such a case, an optional additional analysis may be performed to examine if the subgroups belong to the same class or not. For that purpose, the statistical tests to check differences between subgroups include, but are not limited to, Hotelling's T² or Wilks' Lambda.

F. Certain Variations in the Statistical Analysis

Although in the original derivation of FDA no distributional assumptions were imposed, it has been shown (Johnson & Wichern, 1992) that FDA is an optimal classification procedure in the sense of the smallest misclassification error rates under two assumptions: 1) multivariate normality of the p discriminatory genes, and 2) equal p×p covariance matrices for each of the c classes. Violation of the assumptions affects several aspects of FDA. For instance, unequal covariance matrices significantly affect the appropriate form of the FDA classification rule, so in certain embodiments a quadratic classification rule may be used as an optimal rule rather than the classification rule in Eq. 12 for the analysis of gene and protein expression data. Agreement between the quadratic rule and the linear one in Eq. 12 will decline as the sample sizes decreases, the differences in class covariance matrices increase, the class means become closer, or the number of discriminatory genes increases. In certain embodiments, the effect of violations of the assumptions may be decreased by employing LOOCV coupled with a FDA classifier in the discriminatory gene selection process. In certain embodiments, the Fisher classification rule is equivalent to the classification rule based on minimum total probability of misclassification, when the prior probabilities are equal to 1/c (i.e. the probability of the sample belonging to any one class is equal to any other).

Certain embodiments of the inventive analytical methods described herein employ linear combinations of individual genes as variables in the classifier instead of the individual genes themselves. In embodiments where the cellular state-related genes used for the classifier are chosen based on Wilks' lambda criterion and LOOCV, the number of selected genes is usually still large (50 or more depending on the situation). If all individual genes are considered independently in constructing a classifier (ie., a Bayesian classifier), and new samples are classified using the sum of all gene contributions to the classifier, the classifier may be useful, but in some embodiments, it may not capture the interaction of the genes and may be biased to redundant characteristics. In addition, the parameters in the classifier may be subject to statistical variations of the individual genes. In embodiments where all the genes are considered together as seen in multiple discriminant analysis (MDS), it may be difficult to estimate the model parameters due to the large number of cellular state-related genes and singularity in the data. In embodiments employing the linear combinations of individual genes obtained from FDA, the important discriminating characteristics are often captured at the outset because the algorithm seeks the relevant directions (weights) for separation of classes. Thus, the number of variables used for the classifier is generally reduced to several FDA projection variables (the number of classes—1), while capturing in a large degree the discriminating characteristics in data. This reduction in variables may be achieved with significant accuracy cost in discrimination.

In certain embodiments, the use of FDA in analysis of gene and protein expression also reduces the amount of noise obscuring the information content of the data. Signals that nearly appear to be random noise will be filtered out during the process of obtaining the weights for the linear combinations.

Optionally, the interactions and relative contributions of the individual genes or proteins to the classification can be interpreted from the discriminant weights in the linear combinations, improving the understanding the discriminant features in the data. As a result, the FDA classifier using linear combinations as variables can provide the preferable aspects in classification, including robustness in performance, non-complexity in modeling and improvement in interpretation.

G. Certain Application of the Foregoing Analytical Methods

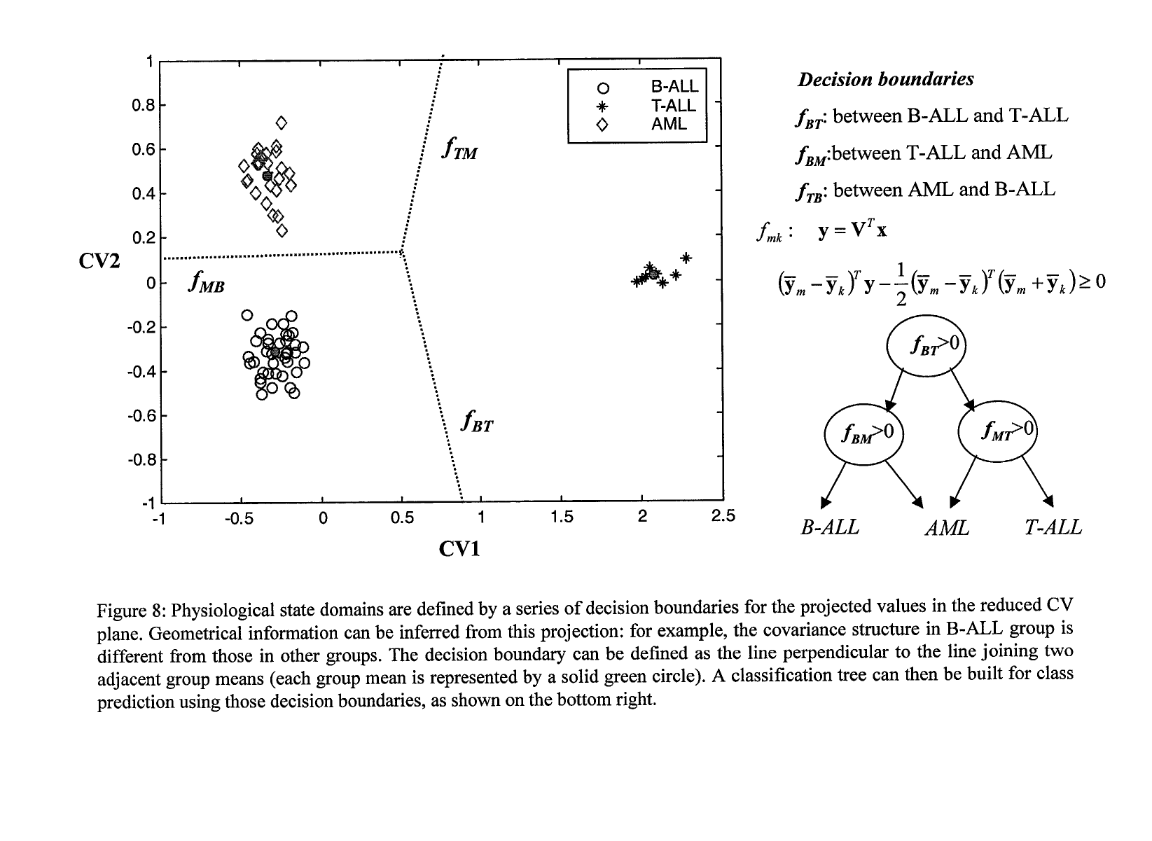

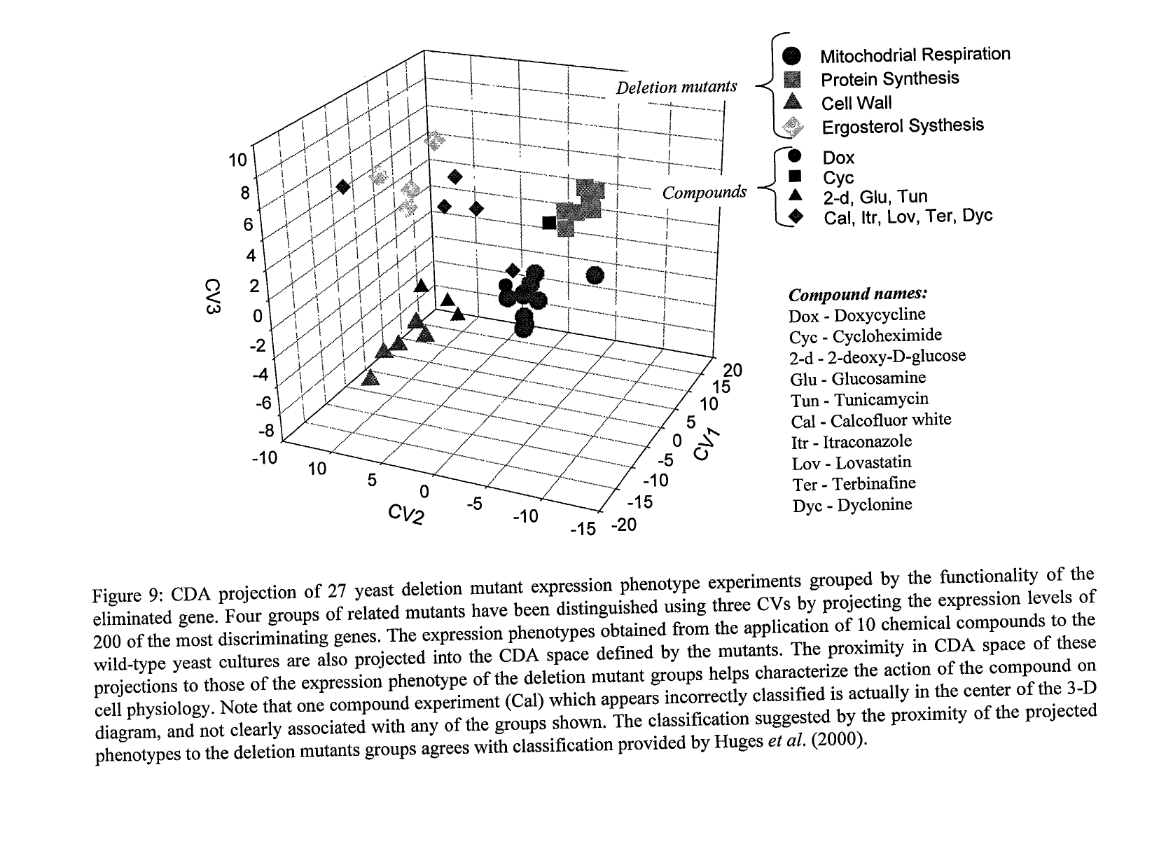

The operation of any of the preceding analytical approaches, in part or in full and optionally in combination with other analytical techniques, provides a powerful methodology for organizing and distilling meaning from gene and protein expression data. In certain embodiments, the variability-based methods provide a systematic method of integrating the information content of the large volumes of data in an expression phenotype. Furthermore, the generation of a reduced dimensional space and the projection of data into this space allows the differentiation of samples from distinct cellular states. As a result, the physiological states can be defined in the reduced dimensional space through a series of equality and inequality constraints for the projection variables FV (see FIG. 8). In further embodiments, projections, by virtue of their ability to group samples from similar physiological states, they are an integral part of classifiers that diagnose the state of a cell or tissue from the measurement of the expression phenotype (FIG. 9). In certain embodiments, such discriminating capability may be applied to situations of medical diagnosis and biotechnological applications. For example, and as decribed in greater detail below, candidate drugs can be screened or bioreactor controls can be pursued such as to bring about a desired change in the cellular state that, in essence, reverses the expression phenotype to that of a normal tissue or establishes a desirable pattern of gene expression that corresponds to high productivity. In some embodiments, the described projections facilitate the implementation of such applications by providing specific means by which the effect of the sum total of genes can be assessed. In many embodiments, the magnitude of discriminant loadings and/or standardized FDA loadings of the expression level of the various genes allow the ranking of the relative importance of each gene in defining the expression phenotype and physiological state. In general, the FDA method employs an a priori classification of samples. Although this may be straightforward in certain cases, in general, this is not a trivial matter. For example, samples may be classified as malignant without any note as to the type of specific cancer involved, or, in production systems, a state of low productivity may reflect more than one-expression phenotypes. Although such heterogeneous samples. will generally produce less well-defined states in their FDA projections, one can take further steps to identify possible subdivisions within a particular physiological class.

In operation, the methods and components for receiving gene or protein expression data, the methods and components for analyzing the gene expression data, and the methods and components for presenting information may involve a programmed computer with the respective functionalities described herein, implemented in hardware or hardware and software; a logic circuit or other component of a programmed computer that performs the operations specifically identified herein, dictated by a computer program; or a computer memory encoded with executable instructions representing a computer program that can cause a computer to function in the particular fashion described herein.

Those skilled in the art will understand that the systems and methods of the present invention may be applied to a variety of systems, including IBM-compatible personal computers running MS-DOS or Microsoft Windows.

The computer may have internal components linked to external components. The internal components may include a processor element interconnected with a main memory. The computer system can be an Intel Pentium®-based processor of 200 MHz or greater clock rate and with 32 MB or more of main memory. The external component may comprise a mass storage, which can be one or more hard disks (which are typically packaged together with the processor and memory). Such hard disks are typically of 1 GB or greater storage capacity. Other external components include a user interface device, which can be a monitor, together with an inputing device, which can be a “mouse”, or other graphic input devices, and/or a keyboard. A printing device can also be attached to the computer.

Typically, the computer system is also linked to a network link, which can be part of an Ethernet link to other local computer systems, remote computer systems, or wide area communication networks, such as the Internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on a mass storage. A software component represents the operating system, which is responsible for managing the computer system and its network interconnections. This operating system can be, for example, of the Microsoft Windows' family, such as Windows 95, Windows 98, or Windows NT. A software component represents common languages and functions conveniently present on this system to assist programs implementing the methods specific to this invention. Many high or low level computer languages can be used to program the analytic methods of this invention. Instructions can be interpreted during run-time or compiled. Preferred languages include C/C++, and JAVA®. Most preferably, the methods of this invention are programmed in mathematical software packages which allow symbolic entry of equations and high-level specification of processing, including algorithms to be used, thereby freeing a user of the need to procedurally program individual equations or algorithms. Such packages include Matlab from Mathworks (Natick, Mass.), Mathematica from Wolfram Research (Champaign, Ill.), or S-Plus from Math Soft (Cambridge, Mass.). Accordingly, a software component represents the analytic methods of this invention as programmed in a procedural language or symbolic package. In a preferred embodiment, the computer system also contains or accesses a database comprising values representing levels of expression of one or more genes or proteins characteristic of a sample. In certain embodiments, the computer system contains or accesses a database comprising a set of discriminator vectors that may be used for the classification of a sample into a class representing a biological state on the basis of gene or protein expression data.

In an exemplary implementation, to practice the methods of the present invention, a user first access data representing gene or protein expression levels in one or more samples. These data can be directly entered by the user from a monitor and keyboard, or from other computer systems linked by a network connection, or on removable storage media such as a CD-ROM or floppy disk or through the network. Next the user causes execution of expression profile analysis software which performs the steps of analyzing gene or protein expression levels. Such software may employ an analysis method that involves determining a measure of variability in any of the various ways described herein. Such software may provide various layers of analysis. For example, in certain embodiments, the software may analyze the data to answer one or more of the following questions: (a) Of the gene(s) and/or protein(s) probed, which ones are particularly relevant to a disease or, in general, a cellular state of interest, by virtue of their ability to characterize a particular cellular state as such; (b) Is there a specific pattern of gene and/or protein expression that marks the occurrence of a particular physiological state; and (c) Can such patterns be used to diagnose the physiological state of cell and tissue samples. In certain embodiments, the software may compare data representing gene and/or protein expression in a sample of unknown cellular state to gene and/or protein expression data from samples of a known cellular state in order to classify the sample of unknown cellular state. Such comparison may be done using the novel methods described herein that employ a measure of variability, or comparisons may, in some cases, be performed using a variety of methods that, in view of this specification, are known in the art, some of which are described below.

6. Other Methods for Analysis of Gene and Protein Data

In certain aspects, the invention provides one or more genes that are related to a particular cellular state or change in cellular state. For example, the invention provides genes of Table 1 that are related to a hyperproliferative state in epithelial cells, genes of Table 2 that are related to different classes of leukemias and genes of Table 3 that are related to polyhydroxyalkanoate production. In some embodiments the invention also provides discriminatory patterns comprising one or more of the above genes or proteins encoded therein. In certain embodiments a different sample may be compared to the genes and patterns described herein, for the purpose of, for example, classifying the sample or evaluating a manipulation of the sample. Such comparisons may employ the variability-based statistics described above, or comparisons may be performed using any of the various statistical methods that, in view of this specification, may be selected by one of skill in the art.

A variety of statistical methods are available to assess the degree of relatedness in expression patterns of different genes. Generally, such statistical methods may be broken into two related portions: metrics for determining the relatedness of the expression pattern of one or more gene, and clustering methods, for organizing and classifying expression data based on a suitable metric (Sherlock, 2000, Curr. Opin. Immunol. 12:201-205; Butte et al., 2000, Pacific Symposium on Biocomputing, Hawaii, World Scientific, p.418-29).

In one embodiment, Pearson correlation may be used as a metric. In brief, for a given gene, each data point of gene expression level defines a vector describing the deviation of the gene expression from the overall mean of gene expression level for that gene across all conditions. Each gene's expression pattern can then be viewed as a series of positive and negative vectors. A Pearson correlation coefficient can then be calculated by comparing the vectors of each gene to each other. Pearson correlation coefficients account for the direction of the vectors, but not the magnitudes.

In another embodiment, Euclidean distance measurements may be used as a metric. In these methods, vectors are calculated for each gene in each condition and compared on the basis of the absolute distance in multidimensional space between the points described by the vectors for the gene.

In a further embodiment, the relatedness of gene expression patterns may be determined by entropic calculations (Butte et al. 2000). Entropy is calculated for each gene's expression pattern. The calculated entropy for two genes is then compared to determine the mutual information. Mutual information is calculated by subtracting the entropy of the joint gene expression patterns from the entropy for calculated for each gene individually. The more different two gene expression patterns are, the higher the joint entropy will be and the lower the calculated mutual information. Therefore, high mutual information indicates a non-random relatedness between the two expression patterns.

The different metrics for relatedness may be used in various ways to identify clusters of genes. In one embodiment, comprehensive pairwise comparisons of entropic measurements will identify clusters of genes with particularly high mutual information. A statistical significance for mutual information may be obtained by randomly permuting the expression measurements 30 times and determining the highest mutual information measurement obtained from such random associations. All clusters with a mutual information higher than can be obtained randomly after 30 permutations are statistically significant.

In another embodiment, agglomerative clustering methods may be used to identify gene clusters. In one embodiment, Pearson correlation coefficients or Euclidean metrics are determined for each gene and then used as a basis for forming a dendrogram. In one example, genes were scanned for pairs of genes with the closest correlation coefficient. These genes are then placed on two branches of a dendrogram connected by a node, with the distance between the depth of the branches proportional to the degree of correlation. This process continues, progressively adding branches to the tree. Ultimately a tree is formed in which genes connected by short branches represent clusters, while genes connected by longer branches represent genes that are not clustered together. The points in multidimensional space by Euclidean metrics may also be used to generate dendrograms.

In yet another embodiment, divisive clustering methods may be used. For example, vectors are assigned to each gene's expression pattern, and two random vectors are generated. Each gene is then assigned to one of the two random vectors on the basis of probability of matching that vector. The random vectors are iteratively recalculated to generate two centroids that split the genes into two groups. This split forms the major branch at the bottom of a dendrogram. Each group is then further split in the same manner, ultimately yielding a fully branched dendrogram.

In a further embodiment, self-organizing maps (SOM) may be used to generate clusters. In general, the gene expression patterns are plotted in n-dimensional space, using a metric such as the Euclidean metrics described above. A grid of centroids is then placed onto the n-dimensional space and the centroids are allowed to migrate towards clusters of points, representing clusters of gene expression. Finally the centroids represent a gene expression pattern that is a sort of average of a gene cluster. In certain embodiments, SOM may be used to generate centroids, and the genes clustered at each centroid may be further represented by a dendrogram. An exemplary method is described in Tamayo et al., 1999, PNAS 96:2907-12. Once centroids are formed, correlation are evaluated by, for example, one of the methods described supra.

8. Exemplary Applications

A. Diagnosing a Neoplasm

The invention further provides a method for diagnosing a neoplasm, e.g., leukemia or an epithelial carcinoma. A neoplasm is diagnosed by examining the expression of at least a discriminatory HPE nucleic acid sequences level of HPE proteins from a test cell population that contain a suspected tumor. The population of test cells may contain the primary tumor, e.g., epithelial tissue, or, alternatively, may contain cells into which the primary tumor has disseminated, e.g., saliva, feces, blood or lymphatic fluid.

The expression of one or more of the HPE sequences is measured in the test cell and compared to the expression of the sequences in the reference cell population. The reference cell population preferably contains at least one cell whose neoplastic state is known. For example, the epithelial cancer stage of the reference cell population is preferably known. If the reference cell contains no neoplastic cells, than a similarity in HPE sequence expression between the test cell population and the reference cell populations indicates that the test cell population likewise does not contain any neoplastic cells. On the other hand, a difference in expression of HPE sequence between the test and reference cell population indicates that the test cell population contains a neoplastic cell.

Conversely, when the reference cell population contains at least one neoplastic cell, a similarity in HPE expression pattern indicates that the test cell population also includes a neoplastic cell. Alternatively, a differential expression pattern indicates that the test cell population contains non-neoplastic cells.

B. Identifying and Categorizing Epithelial Cancer Stage

In addition to providing a means for detecting cancerous epithelia cells, the invention provides a method of categorizing the stage of epithelial cancer in a subject. By “categorizing epithelial cancer stage” is meant the determination of the metastatic stage of the epithelial cancer. In other words, determining whether a subject's epithelial cancer is metastatic as opposed to non-metastatic, or aggressive versus non-aggressive, or it may refer to the receptivity or refractiveness of the cancer to a particular therapeutic regimen.

The method includes providing a cell from the subject and detecting the expression level of one or more of the nucleic acid sequences in Table 1 in the cell. The expression of the nucleic acid sequences is then compared to the level of expression in a reference cell population. In general, test cell expression profiles that are different from a normal and similar to a tumor. Non-metastatic epithelial cancer population are indicative of a metastatic epithelial cancer test cell population. In other embodiments, the reference cell population comprises metastatic epithelial cancer cells. In such an embodiment, a test cell expression profile similar to the reference cell would be indicative of metastatic epithelial cancer whereas a different expression pattern is indicative of nonmetastatic epithelial cancer.

If desired, relative expression levels within the test and reference cell populations can be normalized by reference to the expression level of a nucleic acid sequence that does not vary according to epithelial cancer stage in a subject.

C. Assessing the Efficacy of a Treatment of a Neoplasm in a Subject

The differentially expressed HPE sequences identified herein also allow the course of treatment of a neoplasm, such as leukemia or an epithelial carcinoma, to be monitored. In this method, a test cell population is provided from a subject who is undergoing treatment for a neoplasm. If desired, the test cell population can be taken from the subject at various times before, during, and after treatment. The expression of a discriminatory set of HPE genes in the test cell population is then measured and compared to a reference cell population which includes cells whose neoplastic, i.e., as epithelial carcinomas, stage is known. Preferably, the reference cells have not been exposed to the treatment.

If the reference cell population contains no neoplastic cells, a similarity in expression between the test and reference cell populations indicates that the treatment is efficacious. However, a difference in expression patterns indicates that the treatment is not efficacious.

By “efficacious” is meant that the treatment leads to a decrease in size or metastatic potential of a neoplasm in a subject, or a shift in a tumor stage to a less advanced stage. When the treatment is applied prophylactically, “efficacious” means that the treatment retards or prevents the formation of the neoplasm in a subject.

When the reference cell population contains neoplastic cells, a similar expression pattern indicates that the treatment is not efficacious, whereas a dissimilar expression pattern indicates that the treatment is efficacious.

Efficacy can be determined in association with any method for treating a particular neoplasm.

D. Identifying a Therapeutic Agent Individualized for Treating a Neoplasm

Genetic differences in individual subjects can result in different abilities to metabolize various drugs. An agent that is metabolized in a subject to act as an anti-neoplastic agent can manifest itself by inducing a change in gene expression pattern in the subject's cells from the pattern characteristic of the non-neoplastic state. Thus, the differentially expressed HPE sequences disclosed herein allow for a putative therapeutic or prophylactic anti-neoplastic agent to be tested in a cell population to determine if the agent is a suitable anti-neoplastic agent in the subject.

According to this method of the invention, a test cell population from the subject is exposed to a therapeutic agent. The expression of one or more HPE sequences is then measured.

In some embodiments, the test cell population contains the primary tumor, e.g. an epithelial carcinoma, or a bodily fluid, such as blood or lymph, into which the tumor cell has disseminated. In other embodiments, the agent is first mixed with a cell extract, for example, a liver cell extract, which contains enzymes that metabolize drugs into an active form. The activated form of the drug is then mixed with the test cell population so that gene expression can be measured. Preferably, the cell population is contacted ex vivo with the agent or its activated form.

By “individualized” is meant that the particular therapeutic agent selected takes the differences in genetic makeup of individuals into account by insuring that the selected agent is therapeutic in a particular subject.

Expression of the HPE sequences in the test cell population is then compared with the expression patterns in the reference cell population. Again, the reference cell population contains at least one cell whose neoplastic, i.e., epithelial carcinoma, stage is known. If the reference cell is non-cancerous, similar gene expression patterns indicate that the agent is suitable for treating the neoplasm in that subject. If the patterns are different, then the particular agent is not suitable for treating the neoplasm in a particular subject.

On the other hand, if the reference cell is cancerous, similar sequence expression patterns indicate that the agent is not suitable for the treatment of that subject. Conversely, differential HPE expression indicates that the agent is suitable for the treatment of that subject.

The test agent may be any compound or composition. In some embodiments, the agent may be a compound or composition known to be an anti-cancer agent. In other embodiments, the agent may be a compound or composition not previously known to be an anti-cancer agent.

E. Screening Assays for Identifying a Candidate Therapeutic Agent for Treating or Preventing a Neoplasm

The differentially expressed HPE sequences disclosed herein can also be used to identify candidate therapeutic agents for treating a neoplasm, for example, leukemia or an epithelial carcinoma. This method is based on the screening of a candidate therapeutic agent to determine if it converts an expression profile of HPE genes that is characteristic of a cancerous state to a pattern indicative of a noncancerous state.

In this method, a test cell population is exposed to a test agent or a combination of test agents, either sequentially or simultaneously. The expression of one or more HPE sequence is measured. Next, the expression of the HPE sequences in the test cell population is compared to the expression level of the HPE sequences in a reference cell population that has not been exposed to the test agent.

An appropriate test agent candidate will increase the expression of HPE sequences that are down regulated in cancerous cells and/or will decrease the expression of those HPE sequences that are up regulated in cancerous cells.

In some embodiments, the reference cell population includes cancerous cells. When such a reference cell population is used, an alteration in expression of the nucleic acid sequences in the presence of the test agent from the expression pattern of the reference cell population in the absence of the reagent indicates that the agent is a candidate therapeutic agent for the treatment of a neoplasm.

The test agent or agents used in this method can be a compound(s) not previously described or can be a previously known compound that has not been shown to be an antineoplastic agent.

An agent that is effective in stimulating the expression of underexpressed genes, or in suppressing the expression of overexpressed genes can be further tested for its ability to prevent tumor growth. Such an agent is also potentially useful for the treatment of tumors. Further analysis of the clinical usefulness of a given compound can be performed using standard methods of evaluating toxicity and clinical effectiveness of anti-cancer agents.

F. Categorizing a Neoplasm

Comparison of HPE expression patterns in test cell populations and reference cell populations can be used to categorize neoplasms in a subject. For example, such a comparison can be used to categorize leukemia or an epithelial carcinoma in a subject.

This method includes providing a test cell population containing at least one neoplastic cell from a subject and measuring the expression of one or more HPE sequences in this test cell. The expression of the nucleic acid sequences in the test cell population is compared to the expression of the nucleic acid sequences in a reference cell population comprising at least one cell whose neoplastic state and category is known. A similarity in expression patterns indicates that the cancerous cell in the test cell population has the same neoplastic category as does the reference cell population.

By “category” is meant the neoplastic state of a given neoplasm. In other words, whether the neoplasm is metastatic or nonmetastatic. In the case of metastatic neoplasms, “categorizing a neoplasm” can mean determining the extent of the metastasis.

G. Assessing the Prognosis of a Subject with a Neoplasm

Also provided is a method of assessing the prognosis of a subject having a neoplasm, such as leukemia or an epithelial carcinoma, by comparing the expression of one or more HPE sequences in a test cell population, which contains at least one cancerous cell, to the expression of the sequences in a reference cell population. By comparing the gene expression profiles of one or more HPE sequences, the prognosis of the subject can be assessed.

In alternative embodiments, the reference cell population includes primarily noncancerous or cancerous cells. When the reference cell contains primarily noncancerous cells, an increase in the expression of an HPE sequence that is overexpressed in the metastatic cancer state, or a decrease in the expression of an HPE sequence that is underexpressed in the metastatic state, suggests a less favorable prognosis.

When the reference cell population contains primarily cancerous cells, a decrease in the expression of a HPE sequence that is overexpressed in the metastatic state, or an increase in the expression of a HPE sequence that is underexpressed in the metastatic state, suggests a favorable prognosis.

H. Treating Metastatic Cancer

Also provided is a method of treating metastatic cancer, for example, metastatic epithelial carcinomas, in a patent suffering from or at risk for developing metastatic cancer. By “at risk for developing” is meant that the subject's prognosis is less favorable and that the subject has an increased likelihood of developing metastatic cancer. This method involves the administration of an agent that modulates the expression of one or more HPE sequences to a subject in need of treatment. Administration can be prophylactic or therapeutic.

In one embodiment, this method comprises administering to a subject, an agent that increases the expression of one or more nucleic acid sequences selected from the group consisting of the HPE genes which are down-regulated in Table 1. These HPE sequences are underexpressed in the transformed cells as compared to the normal cells. The subject is treated with an effective amount of a compound that increases the amount the underexpressed nucleic acid sequences in the subject. Administration can be systemic or local, e.g., in the immediate vicinity of the subject's cancerous cells. This agent could be, for example, the polypeptide product of the underexpressed gene or a biologically active fragment thereof, a nucleic acid encoding the underexpressed gene and having expression control elements permitting expression in the carcinoma cells, or an agent which increases the endogenous level of expression of the gene.

In another embodiment, this method comprises administering an agent that decreases the expression of one or more nucleic acid sequences selected from the group consisting of HPE genes which are up-regulated in Table 1. These HPE sequences are “overexpressed” in the cancerous state. Again, the subject is treated with an effective amount of a compound that decreases the amount of the overexpressed nucleic acid sequences in the subject. As discussed above, administration can be systemic or local. Expression can be inhibited in any of several ways known in the art. For example, expression can be inhibited by administering to the subject a nucleic acid that inhibits, or antagonizes, the expression of the overexpressed gene or genes. In one embodiment, an antisense oligonucleotide can be administered to disrupt expression of the endogenous gene or genes. The use of dominant negative mutants of the HPE gene product(s) are also specifically contemplated.

In an alternative embodiment, the patient may be treated with one or more agents which decrease the expression of those HPE sequences that are overexpressed in the transformed state, alone, or in combination with one or more agents which increase the expression of those HPE sequences that are underexpressed in the transformed state.

Administration of a prophylactic agent can occur prior to the manifestation of symptoms characteristic of aberrant gene expression, such that a disease or disorder is prevented or, alternatively, delayed in its progression. Depending on the type of aberrant expression detected, the agent can be used for treating the subject. The appropriate agent can be determined based on screening assays described herein. Determination of an effective amount of a compound is within the ordinary skill of one in this art.

I. Metabolic Engineering

In one aspect, the present invention allows for determining the relationship between gene and protein expression levels and a the output of a metabolic pathway of a cell. Optionally, methods described herein may be used to relate growth conditions (eg. environmental factors), gene or protein expression and the output of a metabolic pathway. In certain embodiments, the method involves supplementing a variable known to play a role in a cell's metabolic pathway such as the nutrient level in a particular medium, measuring gene and/or protein expression levels, and measuring the output of a metabolic pathway. Variability-based methods described throughout the present invention may be used to analyze such gene or protein expression data. Microbes may be manipulated genetically to, for example, increase expression of one or more genes or proteins associated with a desired level of output from a metabolic pathway, and in this manner, the methods described herein may be used to engineer the production of desirable products. The method may be used to enhance the production level of desired products in the medical, specialty chemicals, materials, fuels and environmental areas. As the capabilities of enzymes and cells continue to expand, it is important to note that there is essentially any molecule of commercial interest might be produced by a microbe under some conditions or with the appropriate genetic manipulation, and many novel molecules can be synthesized to meet present and future needs. The issue with all such applications has been one of economics and this is precisely the topic that metabolic engineering is addressing through its focus on the construction of better biocatalysts by molecular biological means. To date, much of the enormous potential of multiple gene modulation has been relatively unexplored in metabolic engineering. This research has the potential to produce a working example that would be easily emulated in many other areas of industrial and medical importance.

In another aspect, the variables that affect production levels of biopolymers are determined through microarray analysis. Biopolymers are important biodegradable products that are being considered for a large array of applications. In this invention the focus is on the synthesis of polyhydroxybutyrate (PHB), but as alluded to above, there is not a molecule of commercial interest that some microbe is not able to produce. Considering that the cost of raw materials contributes approximately 50% of the total manufacturing cost of fermentation processes, a CO₂-based process could have significant advantages over more conventional glucose-based fermentations producing the same products. In certain aspects, the improvement of the productivity of such processes is a goal of metabolic engineering. Furthermore, a potential process based on cyanobacteria for biopolymer production would enjoy the additional advantage of CO₂-fixation and concomitant credits that would contribute to an overall process of considerable commercial interest and environmental impact.

Further, it is noted that PHB is a member of the much broader family of polyhydroxyalkanoates (PHAs) that can also be synthesized in bacterial cells through the supply of various precursor molecules. The methods developed in this invention are equally applicable to the biosynthesis of any member of the PHA family of molecules, should one be determined to possess properties of interest. For instance other polyhydroxyalkanoates include polyhydroxyproprionate, polyhydroxybutyrate, polyhydroxyvalerate, polyhydroxycaproate, polyhydroxyheptanoate, polyhydroxyoctanoate, polyhydroxynonanoate, polyhydroxydecanoate, polyhydroxyundecanoate, polyhydroxydodecanoate and a mixed polymer of one or more of the forgoing polymers.

J. Pharmaceutical Compositions for Treating Neoplasms

In another aspect, the invention includes pharmaceutical or therapeutic compositions containing one or more therapeutic compounds described herein. Pharmaceutical formulations may include those suitable for oral, rectal, nasal, topical (including buccal and sub-lingual), vaginal or parenteral (including intramuscular, sub-cutaneous and intravenous) administration, or for administration by inhalation or insufflation. The formulations may, where appropriate, be conveniently presented in discrete dosage units and may be prepared by any of the methods well known in the art of pharmacy. All such pharmacy methods include the steps of bringing into association the active compound with liquid carriers or finely divided solid carriers or both as needed and then, if necessary, shaping the product into the desired formulation.

Pharmaceutical formulations suitable for oral administration may conveniently be presented as discrete units, such as capsules, cachets or tablets, each containing a predetermined amount of the active ingredient; as a powder or granules; or as a solution, a suspension or as an emulsion. The active ingredient may also be presented as a bolus electuary or paste, and be in a pure form, i.e., without a carrier. Tablets and capsules for oral administration may contain conventional excipients such as binding agents, fillers, lubricants, disintegrant or wetting agents. A tablet may be made by compression or molding, optionally with one or more formulational ingredients. Compressed tablets may be prepared by compressing in a suitable machine the active ingredients in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may be coated according to methods well known in the art. Oral fluid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives. The tablets may optionally be formulated so as to provide slow or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient; and aqueous and nonaqueous sterile suspensions which may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Formulations for rectal administration may be presented as a suppository with the usual carriers such as cocoa butter or polyethylene glycol. Formulations for topical administration in the mouth, for example buccally or sublingually, include lozenges, comprising the active ingredient in a flavored base such as sucrose and acacia or tragacanth, and pastilles comprising the active ingredient in a base such as gelatin and glycerin or sucrose and acacia. For intra-nasal administration the compounds of the invention may be used as a liquid spray or dispersible powder or in the form of drops. Drops may be formulated with an aqueous or non-aqueous base also comprising one or more dispersing agents, solubilizing agents or suspending agents. Liquid sprays are conveniently delivered from pressurized packs.

For administration by inhalation the compounds are conveniently delivered from an insufflator, nebulizer, pressurized packs or other convenient means of delivering an aerosol spray. Pressurized packs may comprise a suitable propellant such as dichlorodifluoromethane, trichlorofluoromethane, dichiorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insuffiator. When desired, the above-described formulations, adapted to give sustained release of the active ingredient, may be employed. The pharmaceutical compositions may also contain other active ingredients such as antimicrobial agents, immunosuppressants or preservatives.

It should be understood that in addition to the ingredients particularly mentioned above, the formulations of this invention may include other agents conventional in the art having regard to the type of formulation in question, for example, those suitable for oral administration may include flavoring agents.

Preferred unit dosage formulations are those containing an effective dose, as recited below, or an appropriate fraction thereof, of the active ingredient.

For each of the aforementioned conditions, the compositions may be administered orally or via injection at a dose of from about 0.1 to about 250 mg/kg per day. The dose range for adult humans is generally from about 5 mg to about 17.5 g/day, preferably about 5 mg to about 10 g/day, and most preferably about 100 mg to about 3g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

The pharmaceutical composition preferably is administered orally or by injection (intravenous or subcutaneous), and the precise amount administered to a subject will be the responsibility of the attendant physician. However, the dose employed will depend upon a number of factors, including the age and sex of the subject, the precise disorder being treated, and its severity. Also the route of administration may vary depending upon the condition and its severity. Determination of the proper dose and route of administration is within the ordinary skill of those familiar with this art.

The invention now being generally described, it will be more readily understood by reference to the following examples, which are included merely for purposes of illustration of certain aspects and embodiments of the present invention, and are not intended to limit the invention.

### EXAMPLES

### Example 1

### Synechocystis Light/Dark Gene Regulation

We applied FDA projections to four examples of gene expression phenotypes generated in our laboratory and also published in the literature. In the first example, cultures of the photosynthetic bacterium Synechocystis sp. PCC 6803 were cultivated through an initial period of 48 hours of growth under light followed by 24 hours of darkness. The cultures where then cycled between light and dark conditions for 100 minutes each (FIG. 4). The expression levels of 88 genes associated with harvesting of light energy and central carbon metabolism were measured at 23 time points (29 total samples, including duplicates) using DNA microarrays. Gill, R. T., E. Katsoulakis, W. Schmitt, G. Taroncher-Oldenburg and G. Stephanopoulos, “Dynamic transcriptional profiling of the light to dark transition in Synechocystis sp. PCC6803,” (submitted) (2000). Total signal to noise ratio of the microarray fluorescence was determined to be c.a. 4.0 indicating that background noise minimally interfered with the fluorescence of hybridized spots. Reproducibility of expression measurements, evaluated from microarray to microarray measurements, as well as from intra-microarray triplicate spots, was 45% suggesting that a 90% difference in fluorescence is reproducible within 95% confidence level Of the 88 total genes considered, 27 discriminatory genes were identified based on their Wilks' lambda measure with a stringent 99% confidence level. Dillon, W.R., and M. Goldstein. Multivariate Analysis, John Wiley & Sons (1984). FIG. 4 shows the projection of the expression phenotype of the 27 Synechocystis discriminatory genes to the FDA-defined 3-D space. Three dimensions were used in this projection to distinguish the four phenotypic classes of growth under the light and dark conditions shown in FIG. 4. The class separation can also be seen in 2-D diagrams of the above canonical variables (FIG. 4c). CVI distinguishes group 2 from the other groups while CV2 separates groups I and 3. Hence, the second CV loadings provide information on the identity of the genes supporting the differences in the cellular processes occurring under light and dark conditions.

### Example 2

### Oral Epithelial Cancer

To help elucidate the genetic and biochemical mechanisms underlying the onset of oral epithelium cancer, the expression phenotype (transcriptome) of oral epithelium was probed using expression microarrays, specifically the Affymetrix HuGeneFL® microarray containing ˜7000 human genes. The accuracy of the measured expression levels has been assessed to be approximately 82%, so that meaningful gene induction and repression differences can be thus monitored. I. Alevizos et al., submitted (2000); H. Ohyama et al., Biotechniques 29, 530-6 (2000). Although microarrays provide a vast amount of information about the state of transcription in cells and tissues, they are best taken advantage of when complemented by appropriate bioinformatic methods for the extraction of useful biological knowledge and the overall upgrade of their information content. We illustrate below the application of two such methods and have succeeded in identifying 45 genes that are strongly correlated with the appearance of malignancy in oral epithelium. The importance of these findings stems from the implication of associated genetic and biochemical mechanisms in oral carcinogenesis that may lead to the definition of new targets for the development of diagnostic tools and therapeutic procedures.

Samples were obtained from 5 patients with oral cancer and immediately snap frozen. Laser capture microdissection (LCM) was used to procure malignant and normal oral keratinocytes. LCM, RNA isolation, T7 linear amplification, probe biotinylation, GeneChip® array hybridization and subsequent scanning were applied as previously described. I. Alevizos et al.; and H. Ohyama et al. Array to array reproducibility was determined by comparing the signals from duplicate microarrays as well as n-tuplicate features on the same microarray. Differences in expression equivalent to less than one copy per cell were detected for 24 transcripts at >99% confidence (p<0.01, unpaired t-test). Copies per cell are calculated using known concentrations of control transcripts, assuming an average transcript length of 1 kb and a population of 300,000 transcripts per cell.

There are several research issues that can be addressed with microarray data, each requiring a particular set of bioinformatic tools. Commonly asked questions include: (a) Of the large number of genes probed, which ones are particularly relevant to a disease or, in general, a cellular state of interest, by virtue of their ability to characterize a particular cellular state as such; (b) Is there a specific pattern of gene expression that marks the occurrence of a particular physiological state; and (c) Can such patterns be used to diagnose the physiological state of cell and tissue samples. Although some answers to the above questions can be obtained by simple visual inspection of a sample's expression levels relative to those of the control, statistical significance is increased by using multiple samples from each class and applying rigorous analysis in identifying discriminatory genes and their characteristic patterns.

It is expected that only a subset of the total number of genes probed by microarrays will be of consequence in distinguishing a physiological state of interest. This is shown schematically in FIG. 1 depicting the expression distribution of two genes A and B in ten samples obtained from two different types (or classes) of tissues, such as normal and diseased. Clearly, while the expression of gene A is sufficiently distinct in the two types, the significant overlap in the expression of gene B for the two classes of samples reduces its value in differentiating one class of tissue from another. As shown in FIG. 1, the ratio of the “within group variance” to the “total variance” (also known as the Wilks' lambda score, R. A. Johnson, D. W. Wichern, Applied Multivariate Statistical Analysis. Prentice Hall, (1992)) can be used as a metric of each gene's class differentiating potential. Since Wilks' lambda score does not follow any known distribution, the transformation shown in FIG. 4a is applied to approximate Wilks' lambda ratio by a univariate F statistic that allows one to identify discriminatory genes with a specified statistical significance. By this approach, 171 genes are identified whose between-group-variance is significantly larger, under the level of significance (α=0.01), than the variance when the ten samples are considered as a single group.

A preferred classification criterion can be obtained by using the error classification rate. R. A. Johnson, D. W. Wichern, Applied Multivariate Statistical Analysis. Prentice Hall, (1992); P. A. Lachenbruch, M. R. Mickey, Technometrics, 10, 1-11, (1968); SAS/STAT User's Guide. SAS Institute Inc., (1989). In this method, a subset of the available samples (the training set) is used to identify the discriminating genes as well as to define a sample classification model. The classification model (see below) is subsequently tested against the samples that were not included in the training set (the test set) and the misclassification rate is calculated for all possible membership configurations of the training and test sets. This procedure is initiated with a classifier that is based on a single (most discriminating) gene and is repeated as more genes (in order of discriminating power based on their F value) are added to the classifier. The misclassification rate would be expected to decrease as more and more genes are added to the classifier, making it more robust. This is exactly what is observed with the expression data of oral epithelium cancer, as shown in FIG. 4b. Clearly, 40-45 genes accurately predict the class of the samples in the test set and, as such, they are deemed a particularly preferred set of discriminatory genes for the classification of the oral epithelium cancerous state.

The misclassification rate is a function of both the sample population size and the number of genes considered. Even with only three samples describing each of the two states (that is, reserving 2 of the 5 samples to test the classifier developed using the other 3), correct classification is achieved over 85% of the time if a sufficient number of genes are considered. Four samples from each group (leave one out case) were sufficient to achieve perfect classification for all permutations of the training and testing sets when at least 45 genes are considered. These results show that accurate classification can be achieved even with only a few samples if a sufficient number of genes are included in the classifier. Furthermore, FIG. 4b shows that consideration of one or two genes as “markers” for disease is an insufficient measure of physiological state. The procedure of cross-validation is discussed further in FIG. 4b.

Table 1 summarizes the discriminatory genes obtained by applying the above procedure to the oral epithelium gene expression data. As an additional validation step of the experimental and computational methods used in deriving these results, we selected three genes from Table 1 whose expressions are consistently altered in the 5 paired cases of oral cancer and applied real-time quantitative PCR (RT-QPCR) to independently measure their expression levels. The three genes were Neuromedin U (interacting protein with G-protein coupled receptors), Wiln's tumor related protein (tumor suppressor) and aldehyde dehydrogenase-10 (xenobiotic enzyme, fatty aldehyde dehydrogenase). Table 4 summarizes the RT-QPCR results of these three genes in the original 5 cases as well as 5 new independent cases of oral cancer. For the three genes identified, a positive comparison between the GeneChip® expression data and RT-QPCR data is observed for more than 80% of the cases examined. I. Alevizos et al., submitted (2000).

Table 4

Validation of 3 discriminatory genes (identified by GeneChip® profiling and bioinformatic analysis) by real-time quantitative PCR (RT-QPCR). Shown are the numbers of cases where statistically significant differences between the control and malignant samples were found in the expression levels of the indicated genes using the two methods. GC=GeneChip® data.

| | Neuromedin U | | WT-1 | | ALDH-10 | |
|---|---|---|---|---|---|---|
| | GC | RT-QPCR | GC | RT-QPCR | GC | RT-QPCR |
| Original 5 Cases | 5/5 | 5/5 | 5/5 | 4/5 | 5/5 | 4/5 |
| 5 New Independent | | 4/5 | | 4/5 | | 5/5 |
| Cases | | | | | | |

Besides expression differences in individual genes for the two types of tissues, discriminating genes can also be used collectively to define a composite index of cell physiology, using Fisher Discriminant Analysis (FDA). W. R. Dillon, M. Goldstein, Multivariate Analysis. Wiley, (1984). FDA defines a new projection space of lower dimensions where the “between class” variance of the various class samples is maximized. The projection space is defined by Canonical Variables (CV) that are linear combinations of the individual gene expressions. X. L. Wen et al., Proceedings Of the National Academy Of Sciences Of the United States Of America, 95, 334-339, (1998); N. S. Holter, et al., Proceedings Of the National Academy Of Sciences Of the United States Of America, 97, 8409-8414, (2000); 0. Alter, P. O. Brown, D. Botstein, Proceedings Of the National Academy Of Sciences Of the United States Of America, 97, 10101-10106, (2000). Both FDA and PCA use the same eigenvalue decomposition procedure to define the linear projection; however, their objective functions are different. R. A. Johnson, D. W. Wichern, Applied Multivariate Statistical Analysis. Prentice Hall, (1992). By maximizing the between group variance and minimizing the within group variance, FDA generates new projection variables (CV) along which the “between group” variance relative to the “within group” variance is maximized. This allows samples of different predefined classes to cluster in distinct areas of the projection space. In cases where the classes are known a priori, the resulting CV's have much more biologically relevant information than principal components calculated through undirected application of PCA.

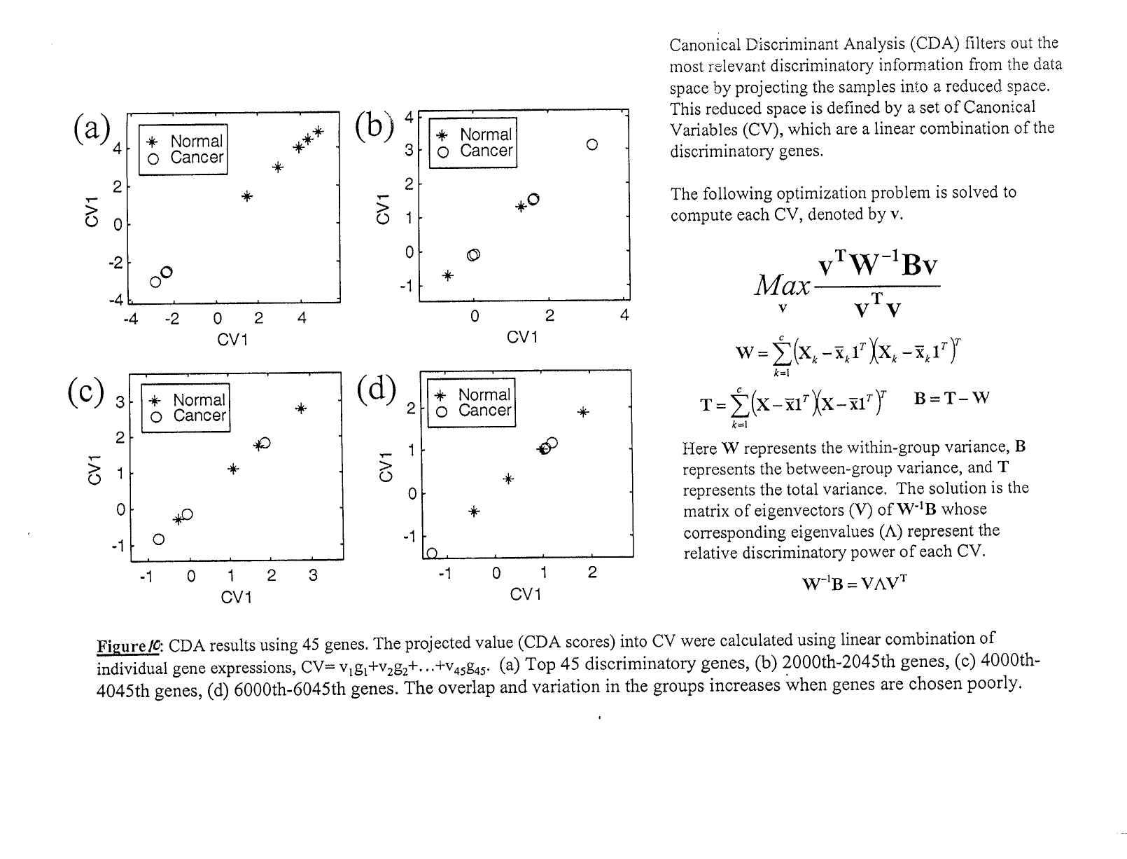

Applying the FDA projection to the expression data from the oral epithelium tissues yielded the two distinct classes shown in FIG. 10, each of them characteristic of the physiological states of normal and malignant oral epithelium. Consequently, the linear combinations of expression data reflected in the canonical variables represent composite metrics that define distinctly the expression phenotype of the corresponding physiological states. These phenotypes, in turn, can be used to classify unknown samples using the expression profiles of the differentiating genes. The classifier employed in the development of the algorithm of FIG. 4 assigned samples to a particular class based on their distance from the mean of the class in the FDA projection space. The reliability of the classification power provided by expression analysis has already been shown in FIG. 4.

A. Discussion of Discriminatory Gene Results

The 45 genes identified by the previous classification schemes exhibit close association with oral cancer development. Two thirds (30) of the genes are downregulated in cancer while 1/3 (15) of the genes are upregulated in cancer. Six of these genes (13%) have been associated with oral cancer either in previous literature (urokinase plasminogen activator (H. Kawamata et al., Int J Cancer 70, 120-7 (1997); S. Nozaki et al., Oral Oncol 34, 58-62 (1998)), cathepsin L (H. Kawamata et al., Int J Cancer 70, 120-7 (1997); P. Strojan et al., Clin Cancer Res 6, 1052-62 (2000)), cytochrome P450 (G. 1. Murray et al., Gut 35, 599-603 (1994)), ferritin light polypeptide (C. Leethanakul et al., Oral Oncol, 36(5), 474-83 (2000)), interleukin 8 receptor beta (B. L. Richards et al., Am J Surg., 174(5), 507-12 (1997)), or by association with chromosomal aberrations found in oral cancers (phospholipase A2). For 39 of the 45 discriminating genes identified by our experimental analysis there is no previously reported chromosomal aberration or differential gene expression. Thus our approach may have identified many candidate genes central to the genesis of oral cancers. Table 1 shows that a number of these genes are members of biological and functional pathways important to tumorigenesis: metastasis and invasion (urokinase plasminogen activator, oncofetal trophoblast glycoprotein, cathepsin L, Wilms tumor related protein, FAT); oncogenes (GRO2, AML1); tumor suppressors (Wilms tumor related protein, FAT); cell cycle and related proteins (heat shock protein 90); signal transducers (crystallin alpha-B) and members of xenobiotic metabolism pathways (aldehyde dehydrogenase-9, aldehyde dehydrogenase-10, carboxylesterase-2, cytochrome p450).

An important objective of this study is to identify genes not previously implicated in cancer and place them into functional pathways or to identify genes with diagnostic and predictive value. The outcome of our study provides data which can generate testable hypotheses. Of particular importance are the differentially expressed genes that are not yet functionally characterized or associated in head and neck/oral carcinogenesis. Neuromedin U (Nmu) is significantly downregulated in 5/5 oral tumors examined. Nmu is a poorly understood protein that manifests potent contractile activities on smooth muscle cells. P. G. Szekeres et al., J Biol Chem 275, 20247-50 (2000). Recently, two G-protein coupled receptors (Nmu1 and Nmu2) have been identified to interact with Nmu with nanomolar potency. R. Fujii et al., J Biol Chem 275, 21068-74 (2000); R. Raddatz et al., J Biol Chem (2000). Our data provide strong evidence that Nmu is relevant in the development of oral malignancy and suggest the need for further study of the role of Nmu (down regulated expression in tumor) in carcinogenesis.

A very interesting finding is the homology of the translocase of outer mitochondrial membrane 34 (TOM34) with the Drosophila melanogaster Hsp70/Hsp90 organizing protein homolog (AF056198). Both TOM34 and Heat Shock 90 Kd (Hsp90) are in the discriminatory gene list and both are upregulated in cancer. Also upregulated in cancer is Heat Shock protein 70 Kd (Hsp70) which is also ranked high in the discriminatory list although it did not make it to the top 45 genes (ranked at #88, well within the α=0.01 confidence limit used in considering the Wilks' lambda criteria). Several cellular signaling proteins require the coordinated activities of the two heat shock proteins Hsp70 and Hsp90 for their folding, oligomeric assembly and translocation. These substrates include several proto-oncogenic serine, threonine and tyrosine kinases such as Raf and Src. C. Scheufler et al., Cell 101, 199-210 (2000); D. F. Nathan, S. Lindquist, Mol Cell Biol 15, 3917-25 (1995). Hsp90 is essential for Raf function in vivo. A. van der Straten, C. Rommel, B. Dickson, E. Hafen, Embo J 16, 1961-9 (1997). Another member of this pathway found in the discriminatory gene list is Lymphocyte Cytosolic Protein 2 (rank #***, again within the α=0.01 confidence limit) (SLP76), (U20058). SLP76 associates with Grb2 adaptor protein and is a substrate for phosphorylation. The concurrent upregulation of TOM34, Hsp90 and Hsp70 and SLP 76 in cancer suggests upregulation of the signal transduction pathway. Interestingly, our analysis identified a tyrosine receptor kinase (HER3), as well as a secreted protein that activates a tyrosine receptor kinase (FGF8), downregulated in the cancer cells. Further studies are needed to deduce which ligand or ligands and which tyrosine kinase receptors are responsible for the hyperfunctional signal transduction pathways.

One of the hallmarks of oral cancer is the decreased host immune reaction to the tumor. We found downregulation of MHC class I polypeptide-related sequence A, (MICA). Receptors for MICA have been identified in many types of T cells, as well as natural killer (NK) cells. In our analysis MICA is downregulated in the tumor samples, suggesting a negative modulation of the immune response against the transformed cells. S. Bauer et al., Science 285, 727-9 (1999).

The discriminatory gene list also reveals a number of known genes, such as HER3 and FAT, that are expressed contrary to tumors at other anatomical sites. It is clear that further experimentation is needed to elucidate the role of such genes. In this regard, this work indicates how bioinformatic analysis of micro array expression data can generate specific hypotheses to be further tested by specifically designed experiments. Such hypotheses are clearly data driven and, as such, define a new approach to scientific research.

### Example 3

### Acute Lymphoblastic Leukemia (ALL) and Acute Myeloid Leukemia (AML)

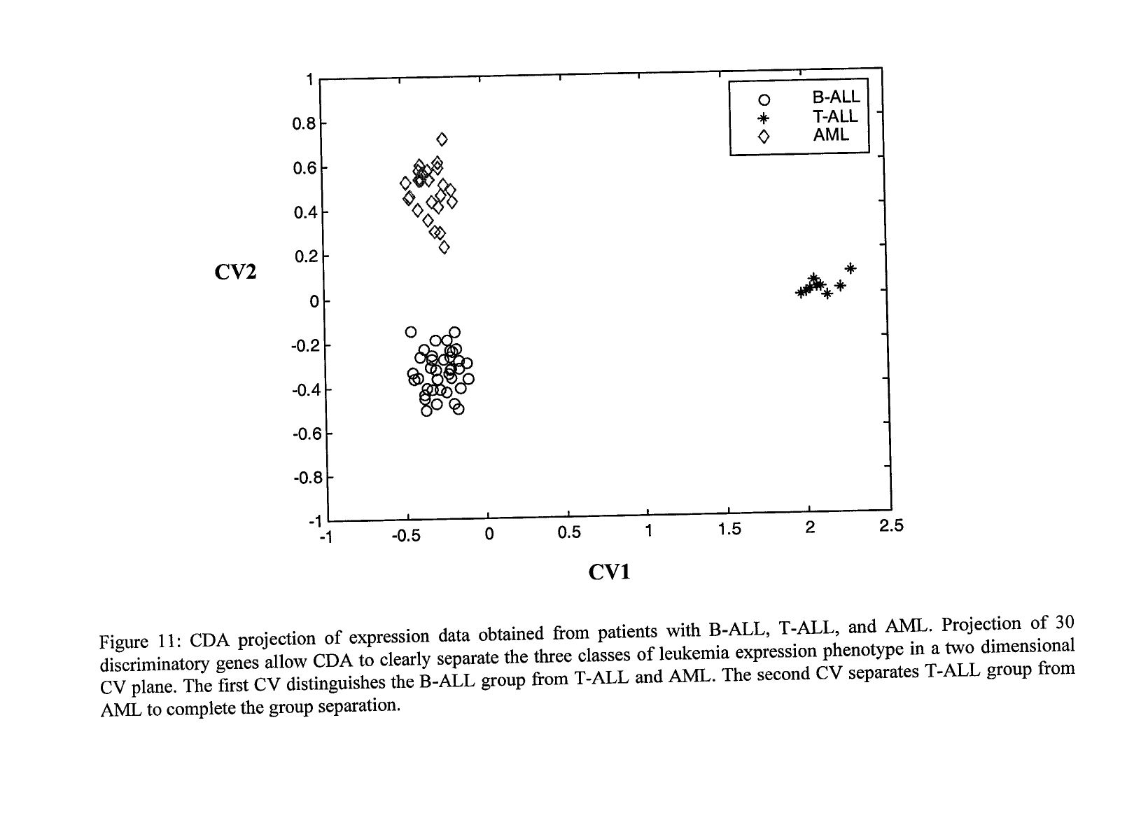

As another example, FDA projections were applied to the expression phenotypes measured in samples from patients with acute lymphoblastic leukemia (ALL) and acute myeloid leukemia (AML). Additionally, the ALL samples were further subdivided into B-lineage ALL and T-lineage ALL (B-ALL and T-ALL, respectively.) Golub, T. R., D. K. Slonim, P. Tamayo, C. Huard, M. Gaasenbeek, J. P. Meslrov, H. Coller, M. L. Loh, J. R. Downing, M. A. Caligiurl, C. D. Bloomfield and E. S. Lander, “Molecular classification of cancer: Class discovery and class prediction by gene expression monitoring.” Science, 286, 531-537, (1999). To reduce the number of genes considered, the Wilks' lambda measure was used again to uncover those genes that provide significant discrimination among the three classes at the 99% confidence level. 1226 genes met this criterion of which 50 genes were selected for use in the FDA projection based on the error rate obtained from leave-one-out cross validation. SASISTA T User's Guide I and 2, SAS Inc. Shown in FIG. 11 are the three groups corresponding to the three leukemia types; since there are three classes of physiology, only two dimensions are required to distinguish the physiological states clearly.

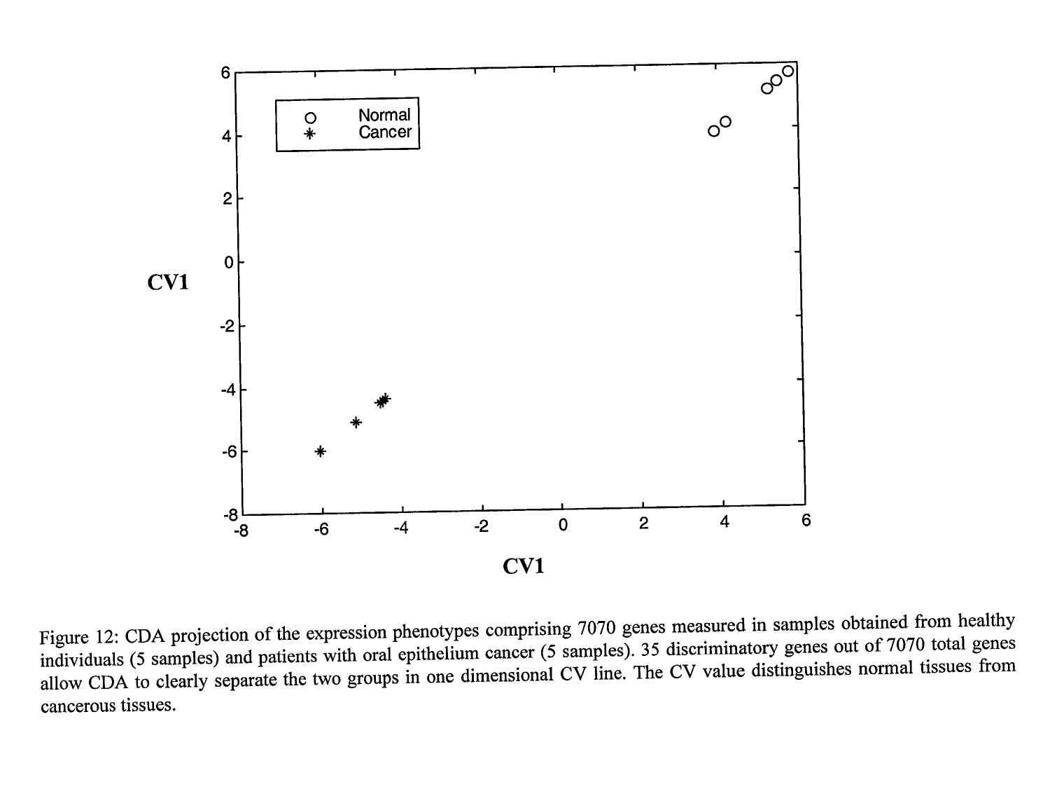

FIG. 12 shows the projection of 35 gene expressions (selected out of 171 most discriminating genes identified at a 99% confidence level from a total of 7070 genes), measured in 10 samples obtained from normal and malignant oral epithelium tissues. Only one FDA dimension is needed to separate the two classes of tissues. Separation is complete, which defines, in the reduced FDA space, the characteristics of oral epithelium malignancy.

The compendium of gene expression data recently published by Rosetta Informatics was also analyzed. Hughes, T. R., M. J. Marton, A. R. Jones, C. J. Roberts, R. Stoughton, C. D. Armour, H. A. Bennett, F- Coffey, 11. Y. Dai, D. D. He, M. J. Kidd, A. M. King, M. R. Meyer, D. Slade, P. Y. Lum, S. B. Stepaniants, D. D. Shoemaker, D. Gachotte, K. Chakraburtty, J. Simon, M. Bard and S. H. Friend, “Functional discovery via a compendium of expression profiles.” Cell, 102, 109-126, (2000). Groups of related single-gene deletion mutants of yeast were identified in this study on the basis of the presumed function of the deleted gene and also by applying a variety of clustering algorithms. By projecting the expression phenotype of four such groups onto the 3-D space defined by FDA, four distinct physiological states are identified describing genetic disruptions in mitochondrial activity, cell wall synthesis, ergosterol synthesis, and protein synthesis (FIG. 9). 200 most discriminatory genes, based on Wilks' lambda criterion were employed in the projection of FIG. 9. The expression phenotypes obtained from wild type cells after treatment with various compounds were also reported in the same study. Using a simple Euclidean distance as a metric of similarity between a drug-treated wild type sample and the mean of a projected group of deletion mutants, the compounds could be easily categorized as “most similar” to a set of related deletion mutants. The projections of FIG. 9 show how the action of a drug causes a nearly equivalent physiological state as a disruption through genetic deletion, providing further support for using the FDA space for a comprehensive definition of the physiological space.

### Example 4

### PHB Accumulation in Synechocystis sp. PCC6803

A. Materials and Methods

Strains Maintenance and Growth Conditions.

Batch cultures of Synechocystis PCC6803 (WT) (Pasteur Culture Collection) were maintained at 30° C. in BG11 medium (SIGMA, St. Louis, Mont.). Throughout each experiment, continuous irradiance of ca. 250 mol photons m₋₂s⁻¹ was provided by cool white fluorescent bulbs. All growth experiments were performed in shake flask cultures in a light-tight incubator (Percival, Perry, Iowa). The limited media used, 0.3% (0.3N) and 10% (10N) of the full BG11 nitrogen level or 10% of the full BG11 phosphate levels (10P), were constituted from their components (SIGMA, St. Louis, Mont.). In addition, identical full and diluted cultures were supplemented with 10 mM Acetate (0.3NA, 10NA, 10PA).

DNA Micro-array Design and Production.

Full-length PCR amplified gene products for nearly every gene in the Synechocystis genome were provided by Dupont Co. PCR products were resuspended in 50% DMSO, spotted using a BioRobotics quill pin micro-arrayer onto Coming GAPS slides (Acton, Mass.), cross-linked using a UV stratalinker, and stored in the dark until use.

RNA Purification.

RNA was purified using Qiagen Mini, Midi, and Maxi kits. Immediately after transfer from the growth culture into 50 ml polypropylene centrifuge tubes, cells were placed into liquid N₂ and chilled to <5° C. within 20 seconds. Chilled cells were immediately centrifuged at 4000× g for 3 minutes in a pre-cooled centrifuge (4° C.), supernatant was discarded, and cell pellets were immediately frozen in liquid N₂ prior to permanent storage at −20° C. Cell pellets were resuspended in buffer RLT (Qiagen) and an equal volume of 0.1 mm glass beads (B.Braun Biotech, Inc., Allentown, Pa.) and ground in a bead-mill (B.Braun) for four cycles of 1 minute grinding and 1 minute on ice. All grinding was performed in a walk-in 4° C. cold room. Lysed cells were then purified following the exact protocols of the Qiagen RNA purification kit. To remove carbohydrates and chromosomal DNA, a final precipitation using 4M LiCl was performed. This modified RNA purification procedure produced results comparable to those previously described for Synechocystis. Mohamed, A., and C. Jansson. 1989. Influence of light on accumulation of phtosynthesis-specific transcripts in the cyanobacterium Synechocystis 6803. Plant Molecular Biology 13:693-700.

cDNA Creation and Labeling.

RNA were reverse transcribed to fluorescently labeled cDNA using 15 U Superscript II/ug RNA, 1× superscript buffer, 1× DTT, 0.5 mM dCTP, dATP, dGTP, 0.2 mM dTTP, and 0.1 mM of Cy-dUTP (Amersham-Pharmacia Biotech, Sweden). Reverse transcription was performed for 2 hours at 42° C. 1.5 11 N NaOH was added and the RNA template was degraded at 65° C. for 10 minutes followed by neutralization with 1 N HCL. Cy3 and Cy5 labeled sample and control cDNA were mixed and ETOH precipitated. Precipitated cDNA was resuspended in 32 1 of pre-warmed (65° C.) hybridization buffer (Clontech) and denatured for 10 minutes at 95° C. prior to applying to the micro-arrays. All micro-arrays utilized the same control RNA in the Cy5 channel. Approximately 1 mg of total RNA was purified from cells in mid-exponential growth phase in full BG11 media in which 3% (v/v) CO₂ in air was bubbled. This control RNA was distributed into 25 μg aliquots and stored frozen until use.

Hybridization and Scanning.

Micro-arrays were denatured for 2 minutes in 95° C. H2O and flash-cooled in −20° C. ETOH. After heat denaturation, labeled cDNA was flash cooled in an ice-slurry and briefly spun at 7000× g to collect evaporated liquid. A small aliquot (1 ul) of cDNA was removed for spectrophotometric diagnostics and the remaining was carefully pipetted over the micro-array. A glass slide was placed over the hybridizaton solution (Clontech) and hybridization was performed in a water bath overnight at 50° C. (as recommended by manufacturer) in water-tight humidified hybridization chambers (Corning, Acton, Mass.). Arrays were washed in an excess of 1× SSC+0.1%SDS for 5 minutes, 0.2× SSC for 3 minutes, and 0.1× SSC for 5 minutes. Cleaned arrays were briefly washed with 1M Ammonium Acetate and immediately spun at 500× g for 4 minutes to remove all salt deposits prior to scanning. Clean slides were scanned using the Axon Instruments GenePix 4000B (Foster City, Calif.).

Micro-Array Data Acquisition, Filtering, and Analysis.

Micro-arrays were quantified using the GenePix Pro software from Axon Instruments. Erroneous spots were manually flagged and removed from the final data set. All micro-array results were filtered to remove any spots in which at least 60% of the signal pixels were not greater by at least one standard deviation than the local background value for both lasers (532 nm, 632 nm). The median pixel ratio of the filtered data for each spot was used for all subsequent analysis. This ratio was adjusted by the median Cy3/Cy5 ratio automatically generated by the GenePix Pro software. Micro-Array Quality Control. Full-genome micro-arrays S/N ratios (Signal/Noise (S/N)=Average [Signal₁−Background_{I}]/σ_{background}) were routinely greater than ten with several arrays greater than thirty. For each spot on the array the S/N ratio was calculated using the spot signal, local background value, and the background variation across the entire slide. The S/N ratio, therefore, represents the number of standard deviations of the background that the spot signal exceeds the local background value.

PHB Quantification.

Between 50-250 ml of cultures in stationary phase were collected by centrifugation (10 minutes at 3200× g, 4° C.). The resulting pellet was washed once in dH₂O and dried overnight at 85° C. The dry pellets were boiled in 1 ml. of concentrated H₂SO₄ for 1 hr., diluted with 4 ml of 0.014M H₂SO₄ and filtered through a PVDF filter (Acrodisc LC13 PVDF, Pall Gelman Laboratory, Ann Arbor, Mich.). Samples were then diluted 10 times with 0.014 M H₂SO₄ and analyzed by HPLC. Karr, D., J. Waters, and D. Emerich. 1983. Analysis of poly-beta-hydroxybutyrate in Rhizobium japonicum bacteroids by ion-exclusion high pressure liquid chromatography and UV detection. Applied Environmental Microbiology 46:1339-1344. Commercially available PHB, processed in parallel with the samples, and crotonic acid (Sigma-Aldrich, St. Louis, Mont.) were used as standards.

Bioinformatic Analysis.

Fisher Discriminant Analysis (FDA) was performed in MATLAB. These two terms are used interchangeably in this text to describe the following mathematical operations. FDA defines a projection from the original to a reduced gene expression space that maximizes the ratio of the variance-between-groups to the variance-within-groups. This is mathematically equivalent to maximizing the mean separation among the various groups or classes in the reduced dimensional space. If there are c classes in the data, the within-group-variance W and the between-group-variance B are defined as:

$$W = {\sum }_{k = 1}^{c} {( {X}_{k} - 1 {\overline{x}}_{k} )}^{T} ( {X}_{k} - 1 {\overline{x}}_{k} ) and B = T - W = {( X - 1 \overline{x} )}^{T} ( X - 1 \overline{x} ) - W$$

where T is the total variation in the gene expression data set. Xₖ and X are data matrices for samples in class k and the entire expression set, respectively. These matrices are organized such that X(i,j) is the expression of gene j in sample i. \overline{x}ₖ is the group mean (1×g) for class k, while \overline{x} is the mean for all the data. It can be proved that the separation between pre-defined groups in a reduced dimensional space is maximized when the space is defined by the eigen vectors of the matrix W⁻¹B. Mathematically, the eigenvalue decomposition of the matrix is given by:

W⁻¹BL=LΛ

The eigenvector matrix (L) defines the dimensions of the reduced space. Each column of L defines an axis or Discriminant Function (DF) of the FDA space. The diagonal entries of the eigenvalue matrix (Λ) are a measure of the discriminant powers of each corresponding DF. The entries in L contain the discriminant weight for each gene. The discriminant weight determines the contribution of each gene in defining the DF. Finally, the projections of the individual samples onto each DF, or the discriminant score, is calculated by:

$${y}_{j} = {xL}_{j} = {\sum }_{i = 1}^{g} {x}_{i} {L}_{ij}$$

where yⱼ is the discriminant score of the actual sample x on the jth DF. In our analysis, we chose Wilks' lambda, defined as the ratio of the determinant of the between-group variance matrix W to the determinant of the total variance matrix T for each gene, to obtain an initial set of discriminatory genes. Wilks' lambda can be transformed into an F-distribution, which allows the selection of discriminatory genes with an appropriate confidence level. These selected genes were ranked by their F value, and the 30 most discriminating genes were chosen for each case. For a more detailed description see Dillon and Goldstein. Dillon, W. R., and Goldstein, M. 1984. Multivariate Analysis: Methods and Applications. John Wiley and Sons. We defined our groups to be those cells grown in identical media conditions and therefore a total of seven groups corresponding to full BG11, 10% Nitrogen, 10% Phosphate, 0.3% Nitrogen+Acetate, 10% Nitrogen+acetate, 10% Phosphate+Acetate, and BG11+Acetate were considered. A total of 26 arrays were run including parallel flasks and replicates of the same RNA.

B. Results

Manipulating Biopolymer Accumulation.

Nutrient limitation (Nitrogen, Phosphate) accompanied by the addition of an external carbon source (acetate) has been shown to alter PHB accumulation levels over a 50-fold range. In this study we used similar growth conditions to those previous described to manipulate PHB accumulation over a 10-fold range (see Table 5). Cells grown in full BG11 media doubled every 40 hrs, grew to a final cell density of 1.5×10⁸ cells/ml, and accumulated PHB to 0.4% of dry cell weight (DCW) at early stationary phase. When full BG11 was supplemented with an additional carbon source, 10 mM Acetate, the growth rate and final cell density were not altered significantly, however, PHB accumulation levels increased approximately to 1% of DCW. Limitation in nitrogen (10%), and (primarily) phosphate yielded further increases in PHB accumulation that was enhanced in the presence of acetate. Interestingly, more severe limitations in Nitrogen (0.3%) resulted in a dramatic reduction in growth rate and final density without a substantial increase in PHB levels. In this study, PHB levels as high as 11% of DCW were obtained in 10PA media even though the average accumulation level in 10PA cultures characterized by micro-arrays was 4.1% DCW.

Micro-Array Validation.

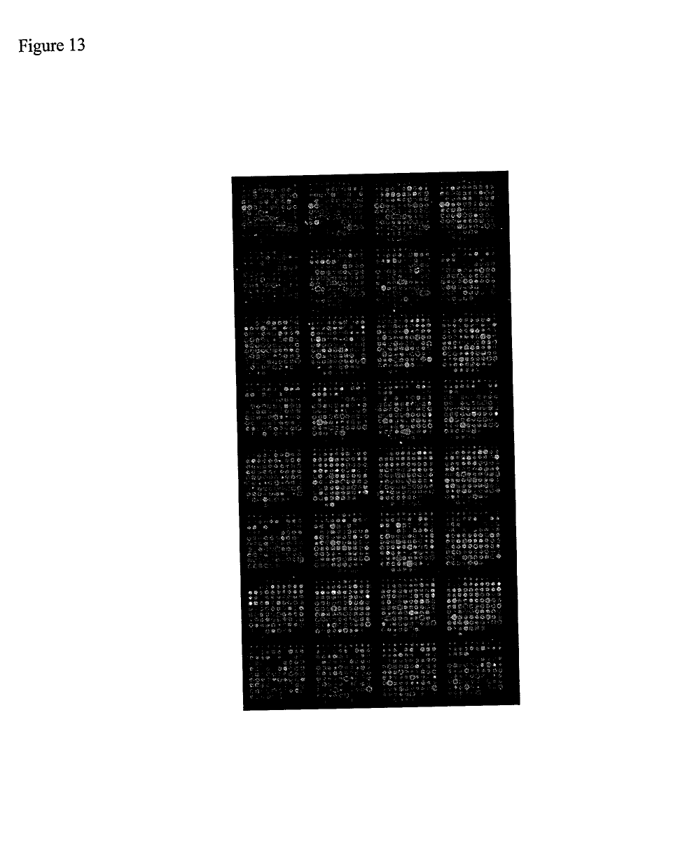

The micro-array protocols used in this study were rigorously validated to ensure reproducibility and obtain a measure of experimental variation. To assess reproducibility, repeat experiments (RNA samples from 3-5 parallel cultures) were performed and analyzed by micro-arrays for which expression ratio means and standard deviations were calculated for each of the genes located on the array. For each gene across the considered samples (3-5 repeats for each of the seven conditions), the measured ratio between the Cy3 and Cy5 labeled samples varied by an average of 36% with a range between 18% (BGA) and 49% (10NA). The expression ratio variance distribution for the sum total of 3169 genes considered is shown in FIG. 13. This value was used to calculate the minimum ratio required for statistically significant expression differences. Using the 95% confidence interval (CI), a transcript ratio difference of 71% (1.96*36%) was determined as the threshold above which the transcript level was deemed to be significantly different in the Cy3 labeled sample relative to the Cy5 labeled sample. Using this value (ratio of 1.71), in addition to growth specific variances, we could accurately determine the significance of a change in a particular gene transcript accumulation level rather than relying upon the standard 2-fold change commonly associated with micro-array data. It is of note however that the variance for these arrays was higher than for other micro-array studies we have performed in which a higher quality RNA was obtained due to higher cellular growth rates (Avg. variance=24%).

Discrimination of Physiological States by Transcriptional Profiling.

A fundamental goal of transcriptional profiling is to associate macroscopic physiological measurements (i.e. growth rate or biopolymer accumulation levels) with gene expression changes. A related concern is to identify those genes which are most discriminatory in defining a physiological state. Often the methodology has been to focus on (i) those genes which exhibit the largest change in accumulation level, or (ii) those gene classes which were previously known to play a role in the process under investigation. Hihara, Y., A. Kamei, M. Kanehisa, A. Kaplan, and M. Ikeuchi. 2001. DNA Micro-array Analysis of Cyanobacterial Gene Expression during Acclimation to High Light. The Plant Cell 13:793-8061; Richmond, C., Glasner, J., Mau, R., Jin, H., and Blattner, F. 1999. Genome-wide expression profiling in Escherichia coli K-12. Nucleic Acids Research 19:3821-3835. What is desired however is a data-driven approach that selects genes that differ most consistently between the states under study while at the same time providing a means for assessing the extent to which physiological changes are reflected at the transcriptional level. In this study we have utilized Fisher discriminant analysis (FDA) to accomplish this objective (see methods).

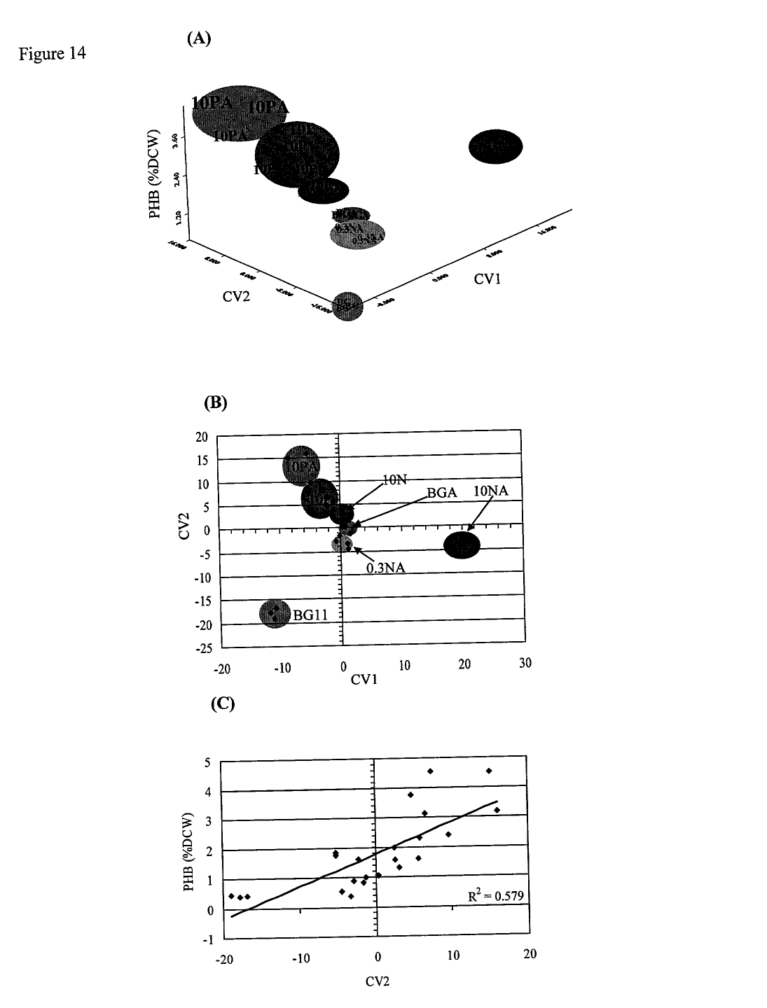

FIGS. 14A and 14B show 2-dimensional FDA projections of the expression phenotypes of cultures grown in different media and exhibiting different levels of biopolymer accumulation. PHB accumulation is plotted as a function of discriminant scores (i.e. weighted linear combinations of discriminatory genes) CV1 and CV2 in FIG. 14A. FIG. 14B is a similar projection showing the clustering of samples obtained from similar cultures. On average, cells grown in full BG11 or 10NA media could most easily be distinguished based on their CV1 and CV2 values. Interestingly, these two samples also exhibited the greatest experimental variability among identical cultures grown in parallel, 44% for BG11 and 49% for 10NA. FIG. 14C shows an important result of this study: PHB accumulation is linearly correlated with CV2, suggesting that PHB accumulation is important to transcriptional differences among the cell states examined.

Identification of Genes Discriminatory of PHB Accumulation Levels.

The Fisher discriminant analysis not only provided a convenient basis for visualizing cells that had accumulated differing levels of biopolymer but also provided a rank order list of genes most discriminatory among the various conditions studied. By examining the weights (Lᵢⱼ) assigned to each gene, we were able to obtain a list of thirty genes which best discriminated among the states measured. These genes are listed in Table 3 along with their unique Cyanobase accession number, proposed function or gene category, and media condition in which they were most significantly altered.

The media condition category was assigned by examining the average and standard deviation for each gene across the conditions studied. A gene was considered discriminatory for a condition when its level in that condition was the furthest away from the mean value. For example, s111632 (10NA) had an average accumulation value (level_{condition}/level_{BG11}) across all conditions of 1.3+/−1.1 with a range of 3.4 (10NA) to 0.6 (10N). The 10NA value was almost 2 SD away from the mean whereas all other values were within 1 SD of the mean.

The majority of the top discriminatory genes were most highly altered in either full BG11 (11 genes) or 10NA (9 genes) growth conditions. The remaining ten genes were spread among the growth conditions of 10N (5 genes), 10PA (2 genes), BGA (2 genes), and 10P (1 gene). Of the top 30 discriminatory genes, 17 had an assigned function-gene category with the remaining of unknown product function. These function-gene categories included genes for chemotaxis, amino acid transport and biosynthesis, cell stress, and cell regulation among others. There was not a clear set of cellular pathways that provided discrimination among the differing growth conditions.

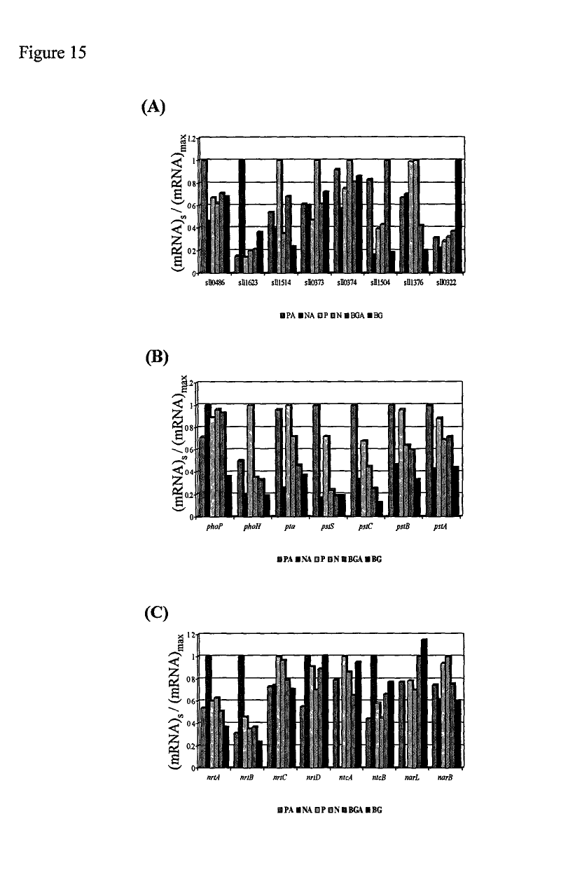

The values for s111376 and s110322 provide a good example of the power of applying FDA to obtaining genes which discriminate among differing cell states (FIG. 15A). In all conditions except full BG11, transcripts for s111376 accumulated. In contrast, s110322 accumulated only in full BG11 conditions. Therefore, the combined level of these two genes is an indicator of growth in full BG11 conditions as opposed to the other conditions studied. Likewise, high values for s110486 indicate phosphate limitation with acetate in the medium while s111623 indicates nitrogen limitation with acetate in the medium.

Nutrient limitation is often the source of considerable stress within the cell. Huckauf, J., C. Nomura, K. Forchhammer, and M. Hagemann. 2000. Stress responses of Synechocystis sp. strain PCC 6803 impaired genes encoding putative alternative sigma factors. Microbiology 146:2877-2889. This was confirmed in our results, where several stress genes were identified among the most discriminatory genes. For example, hsp17 codes for a heat shock chaperone protein and uvrB codes for a protein involved in DNA damage, modification and repair. The hsp17 transcript accumulated four-fold in phosphate limited conditions when compared to full BG11 suggesting a stressful growth environment. In contrast, uvrB transcripts accumulated most substantially when grown in full BG11 media. To further investigate differential stress gene regulation throughout our growth conditions, we examined accumulation levels for each of twelve putative molecular chaperones (dnaKJ, grpE, groELS), three DNA damage related uvr genes (uvrA, uvrC, and uvrD), as well as the SOS-DNA damage regulatory lexA gene (data not shown but available at http:/bioinformatics.mit.edu). In all cases except for groELS (s1r2075-76), there was not any clear pattern of increased stress gene levels among the samples evaluated. However, for the molecular chaperone groELS, phosphate limited cultures produced a 2-3 fold increase compared to stationary phase BG11 cells. The accumulation of these two genes was tightly co-regulated across all samples.

Changes in Phosphate and Nitrogen Related Genes.

Several phosphate related genes accumulated in phosphate limited conditions and differential transcript accumulation within multi-gene families was observed. Specifically, Synechocystis has a phosphate transport system comprised of genes pstABCS from two different multi-gene families. The first family starts with s110680 (pstS) and includes s110681 (pstC), s110682 (pstA), s110683 (pstB), and s110684 (pstB). The second family includes s1r1247-s1r1250 (only one pstB copy). In this study, only the second family (s1r1247-s1r1250) appeared to accumulate preferentially in phosphate limited conditions while the first family (s110680-s110684) did not show any accumulation specific for phosphate limitation (see FIG. 15B for s1r1247-1250; s110680-0684 at http:/fbioinformatics.mit.edu). These results indicate that the s110680-0684 phosphate transport system is not active under moderate phosphate limitation in early to mid stationary phase.

The pho regulon of genes did not clearly differentiate under phosphate limitation. Only transcripts for the phosphate starvation inducible protein phoH significantly accumulated in phosphate limited condition when compared to the other conditions studied. Bhaya, D., D. Vaulot, P. Amin, A. Takahashi, and A. Grossman. 2000. Isolation of regulated genes of the cyanobacterium Synechocystis sp. strain PCC 6803 by differential display. Journal of Bacteriology 182:5692-5699. The phoR and phoB genes encode for a two-component sensory transduction system involved in the response to phosphate limitation. Aiba, H., M. Nagaya, and T. Mizuno. 1993. Sensor and regulator proteins from the cyanobacterium Synechococcus species PCC7942 that belong the the bacterial signal-transduction protein families: implication in the adaptive response to phosphate limitation. Molecular Microbiology 8:81-91; Hirani, T., I. Suzuku, N. Murata, H. Hayashi, and J. Eaton-Rye. 2001. Characterization of a two-component signal transduction system involved in the induction of alkaline phosphatase under phosphate-limiting conditions in Synechocystis sp. PCC 6803. Plant Molecular Biology 45:133-144. These two genes did not substantially accumulate in phosphate limited conditions, however, their maximum levels were observed in 10PA or 10P, respectively. These two genes have previously been observed to affect the alkaline phosphatase gene encoded by s110654. Hirani, T., I. Suzuku, N. Murata, H. Hayashi, and J. Eaton-Rye. 2001. Characterization of a two-component signal transduction system involved in the induction of alkaline phosphatase under phosphate-limiting conditions in Synechocystis sp. PCC 6803. Plant Molecular Biology 45:133-144. This gene did accumulate 3-fold in 10P but only to a lesser extent in 10PA. Transcripts for phoU, phoP, and phoA did not demonstrate any clear trend across the conditions studied.

An interesting result was observed for the phosphotransacetylase gene pta. This gene did show clear transcript accumulation under phosphate limited conditions. The phosphotransacetylase gene product has been reported to be involved in the activation of the PHA synthase enzyme so that control of PHB accumulation primarily resides at the post-transcriptional level. Our results suggest that transcriptional control may also play a role in the accumulation of this biopolymer in Synechocystis.

The results under nitrogen limitation were much less revealing. In fact, of the thirteen nitrogen related genes examined, only three had any clear discriminatory power for nitrogen limited conditions (nrtAB(s111450-51), ntcB). Moreover, those genes were significantly altered only in 10NA conditions but not in 10N conditions. The nrtAB genes are involved in Nitrogen transport and the ntcB gene is a transcriptional activator for nitrogen regulation. Aichi, M., N. Takatani, and T. Omata. 2001. Role of NtcB in activation of nitrate assimilation genes in the cyanobacterium Synechocystis sp. Strain PCC 6803. Journal of Bacteriology 183:5840-5847. Similar to the phosphate transport systems, there are two nitrogen transport systems nrtABCD, located at either s111450-1453 or s1r0040-0044. In contrast to the phosphate system, there was no clear individual family upregulation for these families under nitrogen limited growth conditions. The s111450-51 ntcAB genes did show some preferential accumulation in 10NA conditions, but this outcome was not reflected in s111452-53 or in cells grown in 10N conditions. Interestingly, the icd gene, isocitrate dehydrogenase, was about 2-fold accumulated in nitrogen or phosphate limited cells when compared to full BG11. This gene is known to be positively regulated during Nitrogen starvation and contains a NtcA like promoter which binds the NtcA global regulatory protein. Muro-Pastor, M., J. Reyes, and F. FLorencio. 1996. The NADP+isocitrate dehydrogenase gene (icd) is nitrogen regulated in cyanobacteria. Journal of Bacteriology 178:4070-4076. In contrast, the NtcA regulated rpoD2-V (s111689) sigma factor gene did not show any preferential transcript accumulation in any of the nutrient limited conditions. Muro-Pastor, M., A. Herrero, and E. Flores. 2001. Nitrogen-regulated group 2 sigma factor from Synechocystis sp. strain PCC 6803 involved in survival under nitrogen stress. Journal of Bacteriology 183:1090-1095. We also examined the narL, nitrate/nitrite response regulator, and narB, nitrate reductase, both of which did not show clear accumulation specific for any of the conditions studied.

Finally, we determined those genes that showed the most significant change in nutrient limited compared to full BG11 media (data not shown). Overall, 10PA conditions produced the greatest change (15-fold max. increase) while BGA (maximum of 5-fold) produced the least change in any individual genes transcript accumulation level when compared to full BG11 media. Specifically, transcripts from s111542 (hypothetical protein) accumulated close to 15-fold in 10PA media. Other genes substantially accumulated in 10PA media included s110617 (im30, 12-fold)), s111804 (rps3, 11-fold), s1r2036 (ISY203_a, 10-fold), and s1r2034 (hypothetical, 9.5-fold). Only two genes were consistently accumulated across more than one growth conditions, i) s110617 accumulated 10-12 fold in each of the limited conditions and encodes a 30 kDA chloroplast membrane-associated protein and ii) the extragenic suppressor gene, s111383 (suhB or ssyA), was substantially upregulated in both 10N (7-fold) and 10P (8-fold) and more moderately so in 10PA (5-fold), 10NA (4-fold), and BGA (4-fold). An additional result of interest was a substantial increase (7-fold) in 10NA for s1r1909 which encodes a member of the narL subfamily.

Changes in PHB-Related Genes.

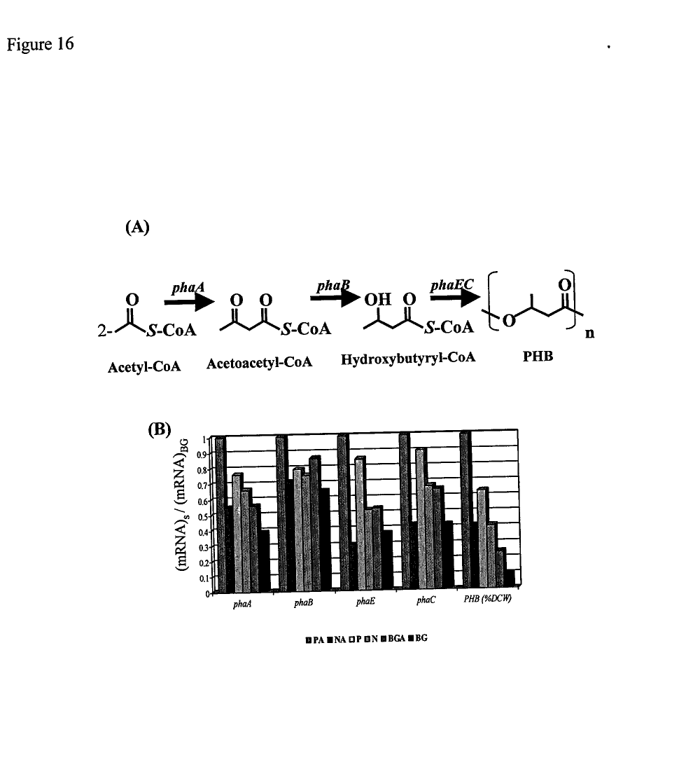

PHB is synthesized in Synechocystis by the combined activities of four gene products from two bi-cistronic mRNA transcripts (FIG. 16). The phaAB (s1r1993 and s1r1994) genes code for the PHA specific b-ketothiolase and acetoacetyl-CoA reductase involved in the first steps of the PHA biosynthetic pathway. Taroncher-Oldenburg, G., K. Nishihara, and G. Stephanopoulos. 2001. Identification of a PHA specific b-ketothiolase and acetoacetylCoA reductase, phaEC, in Synechocystis sp. PCC6803. Applied Environmental Microbiology. The phaEC gene products comprise the PHA synthase which catalyzes the polymerization of hydroxybutyryl-CoA to form PHB. Hein, S., H. Tran, and A. Steinbuchel. 1998. Synechocystis sp. PCC6803 possesses a two-component polyhydroxyalkanoic acid synthase similar to that of anoxygenic purple sulfur bacteria. Archives Microbiology 170:162-170. Transcripts from these genes accumulated preferentially in the conditions of maximum PHB accumulation, 10PA and 10P. This suggests at least some level of control at the transcriptional level for accumulation of the PHB biopolymer. Interestingly, trends within each bicistron (phaAB or phaEC) were remarkably consistent.

C. Discussion

Full Genome Micro-Array Phenotyping.

We have presented transcriptional studies of cyanobacterium Synechocystis using full-genome micro-arrays. Our data have been validated by replicated arrays of the same samples, replicate genes on the same array, and statistical analysis of array to array variability. These analyses allow us to accept expression ratios as low as, for example, 1.7 as being representative of gene differential expression. As pointed out by Tseng et al. (Tseng et al., 2001) such variability needs to be considered separately for each individual gene. By considering many duplicates for each gene at each condition, we maximize our confidence in the results and build a solid foundation for subsequent analysis.

We have shown that dimensional reduction allows the visualization of classes while providing means for identifying and ranking important discriminatory genes. Of particular import is the finding of a linear correlation between PHB level (the physiological parameter of interest in this study) and the value of the second discriminant function (a measure of the collective transcriptional state of the cell). Such relationships form the basis for investigating specific genes in terms of their ability to affect the observed phenotype. In this context, the identified genes may be important targets for directed strain improvements, metabolic engineering, and other manipulations of cellular activity.

A closer examination of the genes in Table 3 demonstrates three categories of genes that are apparent: those such as cheY, encoding a chemotaxis protein expressed as an adaptation to nutrient limitation, but clearly not promising as a target gene for enhancing PHB synthesis in future studies; genes such as the transcriptional regulator hypF, likely to be involved in global cellular responses to nutrient starvation and a potential good candidate for improving PHB synthesis; and unannotated genes such as s110008 for which a function has not been ascribed. The last two categories of genes are potential targets for future metabolic engineering strategies for improving PHB production, which would not have been considered from a mechanistic approach concentrating solely on the enzymatic steps of the PHB synthesis pathway. This demonstrates the value of transcriptional profiling and FDA in target gene identification for metabolic engineering purposes.

DNA Micro-Arrays for High Resolution Phenotyping.

What has become evident as more and more transcriptional profiling studies are reported is that whole genome profiles are remarkably robust classifiers of cell states even though determining gene-function from such studies remains a substantial challenge. In this study, we have used FDA to evaluate our transcriptional profile data and to visualize differences among cells grown in differing media conditions. This analysis revealed that i) specific combinations of gene transcript levels were remarkably strong indicators of cell growth conditions and ii) that a specific combination of transcript levels was a reasonable predictor of biopolymer product accumulation.

One of the most useful outcomes of the FDA was the ability to visualize whole-genome transcriptional profiles in a reduced number of dimensions (FIGS. 14A and 14B). By either increasing the value of CV1 or decreasing the value of CV2, FIGS. 14A and 14B, one is able progress across the gene expression landscape to regions that contain results for cells grown in each of the conditions evaluated. Specifically, each cell state had a different transcript population distribution and could therefore be separated by the levels of those transcripts which differed most significantly. Importantly, each of the samples displayed in FIG. 14 was obtained from separately grown cultures (23 total flasks were evaluated). The fact that samples obtained from cultures grown in identical media were grouped closely together and not grouped closely to cells grown in different media reveals reproducible discrimination at the level of transcriptional regulation. That is, the FDA will only provide discrimination for expression matrices that contain discriminatory structure (genes) as opposed to expression matrices of random structure.

An additionally interesting result was observed for cells grown in either BGA or 10N. Samples from cells grown in these two conditions contained mRNA transcript distributions most central to all of the conditions evaluated. Specifically, CV values for both BGA and 10N samples clustered around the origin. This indicates that in these two samples many of the best discriminatory genes were expressed at low levels thereby providing little magnitude to the cumulative CV1 and CV2 values. An additional explanation could be that the few genes that were discriminatory for these two conditions (5 of top 30 for 10N, 2 for BGA) were poor discriminators with low weights. In this case, even high expression values would not contribute substantially to the CV1 or CV2 values. The end result from either or both of these scenarios would be an overall CV value close to zero (as observed for these samples). This result is also of interest in light of these same samples accumulating intermediate levels of PHB.

The Fisher variable (CV) can be interpreted as the specific combination of gene expression values that maximize the value of the between group variance over within group variance. CV2 in particular can be interpreted to be the specific combination of genes that accounts of the second highest value of the between group variance over within group variance. What is difficult in analyses of these types is assigning a physical interpretation of each of the resulting variables. In the case of CV2, we have shown that PHB accumulation levels are significantly correlated with CV2 values across the 23 samples evaluated. Therefore, one specific statistical interpretation of this result is that changes in PHB levels accurately reflected the second largest feature driving transcript differences among these cell states.

Transcriptional Profiling for Gene-Target Discovery in Metabolic Engineering.

The discovery of genes that collectively correlate with cell states of interest and, therefore, define targets for genetic control is an important focus of metabolic engineering. In this study we have reported the results of a number of different analytical approaches to determining such genes based on whole-genome transcriptional profiles. Specifically, we described results for discriminatory genes as determined by Fisher discriminatory analysis, phosphate related genes, nitrogen related genes, PHB biosynthesis genes, and for those genes which altered most dramatically in each of the conditions studied.

The conditions examined in this study were designed to allow for transcriptional profiling of cell states moderately limited in the different nutrients (starting at N or P sufficient conditions (10% (v/v)) and grown to early stationary phase) that are associated with PHB accumulation. Miyake, M., K. Kataoka, M. Shirai, and Y. Asada. 1997. Control of poly-beta-hydroxybutyrate synthase by acetyl phosphate in cyanobacteria. Journal of Bacteriology 179:5009-5013. At more dramatic starvation conditions, cell growth rate is reduced and final cell density is decreased to a level in which mRNA quality was not suitable for profiling by micro-arrays. Collier, J. Grossman, A. A small polypeptide triggers complete degradation of light-harvesting phycobiliproteins in nutrient-deprived cyanobacteria. EMBO J 13:1039-1047. Therefore, while our studies did force PHB accumulation by nutrient limitation, starvation conditions and the starvation response were not specifically examined. Moreover, in all cultures except 0.3%N we did not observe any chlorosis at the time of mRNA purification. Importantly, the lack of severe starvation conditions combined with the presence of differential PHB accumulation allowed for a clearer analysis of genes involved with biopolymer accumulation rather than cell starvation.

Overall, the use of FDA was shown to provide a concise list of genes which clearly discriminated for particular growth conditions. Similar results were not obtained when evaluated phosphate or nitrogen related genes. In fact, nitrogen related genes did not appear to reflect growth conditions in the majority of cases. The examination of genes which accumulated most dramatically in each of the conditions studied did not provide any useful information with regard to discrimination between PHB accumulation states. In contrast, PHB related genes did vary closely with PHB accumulation suggesting some level of transcriptional control.

The values for s110373 and s110374 suggest a future potential for metabolic engineering of Synechocystis to improve biopolymer accumulation (Table 5).

TABLE 5
Summary of growth conditions and PHB accumulation. BG11 corresponds
to full BG11 growth medium. 10N or 10P corresponds to full BG11 media
limitied in Nitrogen or Phosphate to 10% of the full media level,
respectively. The + acetate refers to media conditions in which 10 mM
acetate was added. PHB (% DCW) refers to the amount of PHB in the
cells as a percentage of dry cell weight at the time of harvest. Averages
and standard deviations from a minimum of three samples were included.

| Condition | % DCW |
|---|---|
| BG11 | 0.4 +/− .04% |
| BGA | 1.0 +/− .03% |
| 10% Nitrogen | 1.7 +/− .2% |
| 10N + Ace | 1.7 +/− .1% |
| 10% Phosphate | 2.6 +/− .7% |
| 10P + Ace | 4.1 +/− .8% |
| 0.3% N + Ace | 0.8 +/− .4% |

Each of these genes was determined, by FDA, to discriminate for nitrogen limited growth conditions. These genes are separated by a 78 bp region on the Synechocystis chromosome and code for a gamma-glutamyl phosphate reductase, the proA gene product, involved in amino acid biosynthesis and a branched chain amino acid transporter like protein. The combined upregulation of these two genes specifically in nitrogen limited conditions suggests a link between amino acid transport and metabolism under nitrogen limited conditions. Interestingly, similar links have been previously made in Synechocystis. Stephan, D., H. Ruppel, and E. Pistorius. 2000. Interrelation between cyanophycin synthesis, L-arginine catabolism and photosynthesis in the cyanobacterium Synechocystis sp. strain PCC 6803. Z Naturforsch 55:927-942. Future studies aimed at supplementing nitrogen limited growth media with amino-acids to offset any amino-acid limitation as well as overexpressing or deleting specific amino acid biosynthesis genes could be envisioned as strategies for altering biopolymer accumulation.

Similar metabolic engineering strategies can be envisioned based on the results for phosphate related genes. In particular, we know that the phosphate limitation is reflected at the transcriptional level in only one of the phosphate transport systems while the second transport system was not substantially altered in the conditions studied. This second transport system, therefore, is potentially available for genetic manipulations aimed at improving growth in phosphate limited cultures by increasing phosphate transport. An additional target for genetic manipulation was the pta gene. This gene is known to be involved in activation of the PHB synthase enzyme and its transcripts were observed to accumulate in correlation with PHB accumulation conditions. Therefore, altering the level of this gene through overexpression or other means is proposed to have an affect of PHB accumulation.

The analysis of nitrogen related genes did not produce any clear targets for future genetic manipulation. These results suggested that the majority of control of the nitrogen response does not occur at the transcriptional level or, more likely, that the conditions studied were not sufficiently limiting to initiate regulatory programs at the gene level. Given the lack of response dynamics of well characterized nutrient starvation genes (i.e. nblA), the absence of chlorosis at the time of harvest, and only minor differences in growth rate, it is likely that cells were not substantially nutrient starved. Baier, K., S. Nicklisch, C. Grunder, J. Reinecke, and W. Lockau. 2001. Expression of two nblA-homologous genes is required for phycobilisome degradation in nitrogen starved Synechocystis sp. PCC6803. FEMS Microbiology Letters 195:35-39; Richad, C., G. Zabulon, A. Joder, and J. Thomas. 2001. Nitrogen or sulfur starvation differentially affects phycobilisome degradation and expression of the nblA gene in Synechocystis strain PCC 6803. Journal of Bacteriology 183:2989-2994.

The results for phaAB and phaEC in nitrogen limited conditions also presented an opportunity for further study and possible metabolic engineering. Specifically, in nitrogen limited cultures PHB biopolymer accumulated to close to 2% (DCW) even though transcript levels were not significantly altered from that of full BG11 media in which PHB accumulated to only 0.4% (DCW). Therefore, genetic manipulations aimed at increasing the levels of the PHB biosynthetic genes in Nitrogen limited growth conditions (among others) present a worthwhile opportunity for improving biopolymer accumulation.

### Conclusion

The FDA projections can be used in a number of different ways. First, as mentioned before, they provide a systematic method of integrating the information content of the large volumes of data in the expression phenotype. Furthermore, this projection also allows the differentiation of samples from distinct physiological states. As a result, the physiological states can be defined in the FDA space through a series of equality and inequality constraints for the projection variables CV (see FIG. 8). Second, by virtue of their ability to group samples from similar physiological states, the FDA projections are an integral part of classifiers that diagnose the state of a cell or tissue from the measurement of the expression phenotype, as suggested by the Rosetta Informatics example (FIG. 9). This is extremely important in situations of medical diagnosis and biotechnological applications as well. In either case, candidate drugs can be screened or bioreactor controls can be pursued such as to bring about a desired change in the physiological state that, in essence, reverses the expression phenotype to that of a normal tissue or establishes a desirable pattern of gene expression that corresponds to high productivity. While these concepts have been suggested before as possible applications of microarray measurements, the described FDA projections facilitate their implementation by providing specific means by which the effect of the sum total of genes can be assessed. Third, the magnitude of discriminant loadings and standardized FDA loadings of the expression level of the various genes allow the ranking of the relative importance of each gene in defining the expression phenotype and physiological state. Discriminant loadings can be calculated by multiplying diagonal element matrix of the total sample covariance matrix (S) and the correlation matrix (R) together into FDA loadings. It should be noted that the FDA method requires that a priori classification of samples be provided. Although this was rather straightforward for the cases presented, we note that, in general, this is not a trivial matter. For example, samples may be classified as malignant without any note as to the type of specific cancer involved, or, in production systems, a state of low productivity may reflect more than one-expression phenotypes. Although such heterogeneous samples. will generally produce less well-defined states in their FDA projections, one can take further steps to identify possible subdivisions within a particular physiological class. Although FDA tries its best to separate groups from one another, if there are subgroups in a particular physiological group, FDA will produce the separated subgroups for that physiological group. In such a case, we have to examine if they belong to the same class or not. For that purpose, the statistical tests to check differences between subgroups include Hotelling's T² or Wilks' Lambda.

Clearly, not all genes present in the expression phenotype are equally important in defining the corresponding physiological state. Although the projection works well with all genes, the inclusion of unrelated genes is bound to increase the noise and make the boundaries of the physiological states more diffuse. Selection and use of the most discriminatory genes yields sharper boundaries among classes.

Although the expression phenotype is an ample measure of the cellular state, it is by no means a complete one. Events catalyzed by the environment may interfere with the translation process ultimately yielding variations in the proteomic and metabolic state of a cell. It is unclear at this point to what extent such variations affect the cellular physiological state. They can nevertheless be handled by the same projection approach described herein and will be investigated as soon as a comprehensive set of such data becomes available. In this way, the use of projections to describe physiological state is as flexible as the data available and will become even more applicable as the amounts of data available increase.

One of the primary limitations in any metabolic engineering study is the selection of target genes for manipulation. One consistent outcome of such studies is the complexity of cell regulatory responses to our attempts to engineer metabolism. Transcriptional profiling has gained considerable attention as a means for target discovery in metabolic engineering among others (i.e. drug discovery). What has been unclear is the extent to which changes in transcript accumulation represent effects of physiological alterations as opposed to causing physiological alterations. In the absence of massive numbers of samples, this information can not be reasonably obtained in transcriptional profiling studies. What can be obtained, however, is a set of target genes which appear to 1) be important to the condition under study and 2) show substantial regulation at the transcriptional level. An additional criteria of importance is determining which genes are be coordinately overxpressed as a regulon to ensure proper ratios of their products in engineered cells. Finally, protein expression studies are required to fully characterize the extent to which the gene-products are also differentially regulated. The genes described in this study provided a reduced set of targets when compared to the whole genome but a more detailed set when compared to strictly looking at the PHB biosynthetic pathway alone. This was demonstrated by comparing the results from the FDA to the results for the phosphate-related, nitrogen-related, and PHB synthesis genes. The FDA genes were all clearly discriminatory for specific nutrient conditions. Also, most of these genes were of no clear relation to the biosynthesis of PHB. As a result, a new target gene set was obtained which satisfied the criteria listed above and which could not have reasonably been obtained otherwise. The phosphate-related and PHB-related genes also showed some promise in terms of target selection even though any nitrogen-related gene targets were not obvious. We can reasonably conclude that studies such as these should rely upon data-driven approaches, such as FDA, for target discovery but can also benefit from an analysis of genes known to be involved in the pathways of interest.

Incorporation by Reference

All of the patents and publications cited herein are hereby incorporated by reference.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method for use in the analysis of gene or protein expression information comprising,
(a) accessing gene or protein expression data comprising expression levels of G genes or proteins in S samples, where the S samples may be classified into C classes representing cellular states;
(b) determining a measure of the variability of expression levels of each gene or protein in the data as a whole; and
(c) determining a measure of the variability of expression levels of each gene or protein within each class of sample.

2. The method of claim 1, further comprising:
(d) determining a between group measure of variability by determining the difference between the measure of variability determined in (c) from the measure of variability determined in (b).

3. The method of claim 1, further comprising generating a comparison of the measure of variability determined in (b) to the measures of variability determined in (c).

4. The method of claim 2, further comprising generating a comparison of the measure of variability determined in (c) to the measure of variability determined in (d).

5. The method of claim 1, wherein the comparison comprises determining the ratio of the measure of variability of (c) to the measure of variability of (b).

6. The method of claim 2, wherein the comparison comprises determining the ratio of the measure of variability of (c) to the measure of variability of (d).

7. The method of claim 3, wherein the comparison comprises calculating a Wilks' lambda score.

8. The method of claim 3, wherein the comparison comprises scaling the measure of variability of (b) by the measure of variability of (c).

9. The method of claim 1, wherein the measure of variability is selected from the group consisting of: variance and kurtosis.

10. The method of claim 1, wherein G is one.

11. The method of claim 1, wherein G is two or greater.

12. The method of claim 1, wherein C is two or greater, and wherein S is equal to or greater than C.

13. The method of claim 1, wherein the data is organized into a data matrix Xₖ for each class k, and wherein each data matrix is organized such that X(i,j) is the expression of gene j in sample i.

14. The method of claim 2, wherein the measure of variability determined in (c) is represented by a matrix B, and wherein the measure of variability determined in (d) is represented by a matrix W.

15. The method of claim 14, wherein W is generated according to the formula
$$W = {\sum }_{k = 1}^{c} {( {X}_{k} - 1 {\overline{x}}_{k} )}^{T} ( {X}_{k} - 1 {\overline{x}}_{k} )$$
wherein \overline{x}ₖ is the group mean (1×g) for class k.

16. The method of claim 14, wherein B is generated according to the formula
B=T−W=(X−1\overline{x})^{T}(X−1\overline{x})−W
wherein \overline{x}ₖ is the group mean (1×g) for class k, \overline{x} is the mean for all the data, and T is the total variance of all the data.

17. The method of claim 14, comprising generating a comparison of the matrix B and the matrix W.

18. The method of claim 17, wherein the comparison is a matrix W⁻¹B.

19. The method of claim 17, further comprising maximizing the separation between the classes in a reduced dimensional space.

20. The method of claim 19, wherein maximizing the separation between the classes in a reduced dimensional space comprises generating an eigenvector matrix L of the matrix W⁻¹B and an eigenvalue matrix Λ of the matrix W⁻¹B.

21. The method of claim 20, wherein a column of L defines a discriminant function of the reduced dimensional space, and wherein each entry in the column indicates the contribution of each gene to the discriminant function.

22. The method of claim 19, wherein the variance-covariance structure is similar in each class.

23. The method of claim 19, wherein maximizing the separation between the classes in a reduced dimensional space comprises generating a singular value decomposition of the matrix W⁻¹B.

24. The method of claim 23, wherein generating a singular value decomposition of the matrix W⁻¹B is performed according to the formula:
W⁻¹B=UΛL^{T}
wherein U is a left singular vector, L is a matrix of discriminant functions, and Λis a matrix of singular values representing the discriminant loadings in the corresponding functions.

25. The method of claim 21, further comprising calculating a discriminator vector for each sample i, wherein the discriminator vector represents a position of the sample in the reduced dimensional space.

26. The method of claim 25, wherein calculating a discriminator vector comprises operating the formula:
$${y}_{j} = {iL}_{j} = {\sum }_{z = 1}^{g} {i}_{z} {L}_{ij}$$
wherein yⱼ is the discriminator score of the sample i (a sample of g genes) for each column j of matrix L, and wherein the discriminator vector is a combination of each yⱼ into a vector having a dimensionality that is equal to the number of dimensions in the reduced dimensional space.

27. The method of claim 3, further comprising generating discriminant loadings based on the comparison.

28. The method of claim 4, further comprising generating discriminant loadings based on the comparison.

29. The method of claim 27, further comprising generating a discriminator vector for each sample, wherein the discriminator vector describes a point in a space having one or more dimensions.

30. The method of claim 28, further comprising generating a discriminator vector for each sample based on the comparison, wherein the discriminator vector describes a point in a space having one or more dimensions.

31. The method of claim 27, further comprising determining the contribution of the expression levels of a gene or protein to the discriminant loadings, wherein a gene or protein that contributes significantly to a dimension is a gene or protein that is related to a cellular state of one or more sample.

32. The method of claim 28, further comprising determining the contribution of the expression levels of a gene or protein to the discriminant loadings, wherein a gene or protein that contributes significantly to a dimension is a gene or protein that is related to a cellular state or a change in cellular state of one or more sample.

33. The method of claim 31, wherein C is two or greater and the space has C-1 dimensions.

34. The method of claim 32, further comprising generating a rank order list of genes or proteins based on contribution to the dimensions of the space.

35. The method of claim 34, wherein the rank order list is generated by comparing the F score for each gene or protein.

36. A method for identifying a gene or protein, the expression of which is related to a cellular state or a change in cellular state comprising,
(a) accessing gene or protein expression data comprising expression levels of G genes or proteins in S samples, where the S samples may be classified into C classes representing cellular states;
(b) determining a measure of the variability of expression levels of each gene or protein in the data as a whole;
(c) determining a measure of the variability of expression levels of each gene or protein within each class of sample; and
(d) identifying a gene or protein which is related to a cellular state or a change in cellular state by identifying a gene or protein for which the measure of variability determined in (c) is less than the measure of variability determined in (b) with a 90% degree of confidence.

37. The method of claim 36, wherein C is two or greater.

38. The method of claim 36, wherein the measure of variability is selected from the group consisting of: variance and kurtosis.

39. The method of claim 36, wherein (d) comprises performing a Fisher Discriminant Analysis.

40. The method of claim 36, further comprising identifying a plurality of genes or proteins according to (d), and wherein the plurality of genes or proteins is a gene or protein group related to a cellular state or a change in a cellular state.

41. The method of claim 36, wherein the gene or protein group is analyzed by determining the contribution of each gene or protein of the group to the power of the group to discriminate between two or more classes of sample.

42. The method of claim 41, wherein determining the contribution of a gene or protein of interest comprises,
(i) generating a subgroup by omitting the gene or protein of interest from the group; and
(ii) testing the power of the subgroup to discriminate between two or more classes of sample.

43. The method of claim 41, wherein determining the contribution of a gene or protein comprises, performing a leave one out cross-validation.

44. A method for identifying a gene or protein expression pattern that is useful for discriminating between samples of two or more cellular states comprising,
(a) accessing gene or protein expression data comprising expression levels of G genes or proteins in S samples, where the S samples may be classified into C classes representing cellular states;
(b) determining a measure of the variability of expression levels of each gene or protein in the data as a whole;
(c) determining a measure of the variability of expression levels of each gene or protein within each class of sample;
(d) generating for each gene or protein a comparison of the measure of variability determined in (b) to the measures of variability determined in (c); and
(e) selecting from among the genes or proteins of (d) a set of genes or proteins and corresponding expression levels that discriminate between two or more classes of sample with a misclassification rate less than 40%, wherein the set of genes or proteins and corresponding expression levels is a pattern that is useful for discriminating between samples of two or more cellular states.

45. The method of claim 44, wherein the pattern is useful for discriminating between cellular states selected from the group consisting of:
(a) hyperprliferative and non-hyperproliferative epithelial cells
(b) AML, B-ALL and T-ALL; and
(c) a bacterium producing higher levels of a metabolite and a bacterium producing lower levels of a metabolite.

46. A computer product for use in analyzing gene or protein expression data, the product disposed on a computer readable medium, and comprising instructions for causing a processor to:
(a) determine a measure of the variability of expression levels of a gene or protein in gene or protein expression data comprising expression levels of G genes or proteins in S samples, where the S samples may be classified into C classes representing cellular states;
(b) determining a measure of the variability of expression levels of the gene or protein within each class of sample in the data.

47. The computer product of claim 46, further comprising instructions for causing a processor to generate for the gene or protein a comparison of the measure of variability determined in (b) to the measures of variability determined in (c).

48. A system comprising a processor and instructions for causing a processor to:
(a) determine a measure of the variability of expression levels of each gene or protein in gene or protein expression data comprising expression levels of G genes or proteins in S samples, where the S samples may be classified into C classes representing cellular states;
(b) determining a measure of the variability of expression levels of each gene or protein within each class of sample in the data.

49. A method for use in modifying the production of a metabolite in a cell comprising:
(a) accessing data comprising a representation of the expression levels of G genes or proteins in S samples, wherein the S samples may be classified into C classes representing biological states, and wherein at least two of the biological states differ in the level of the metabolite that is produced; and
(b) identifying a discriminating gene or protein, the expression levels of which are discriminatory in defining a biological state of higher metabolite production from a biological state of lower metabolite production.

50. The method of claim 49, further comprising identifying a discriminating pattern of gene or protein expression levels.

51. The method of claim 50, further comprising selecting a cell having a desired level of metabolite production by identifying a cell having a pattern of expression levels that is mathematically similar to the discriminating pattern.

52. The method of claim 49, further comprising modulating the expression of the candidate gene or protein in a cell.

53. The method of claim 49, wherein identifying a gene or protein comprises:
(i) determining a measure of the variability of expression levels of each gene or protein in the data as a whole; and
(ii) determining a measure of the variability of expression levels of each gene within each class of sample.

54. A method for use in modifying the production of a polyhydroxyalkanoate in a cell comprising altering the genetic makeup of the cell so as to cause the cell to have a modified expression of a gene represented by an index number selected from the group consisting of: s110008, s110010, s110039, s110322, s110361, s110373, s110374, s110379, s110385, s110396, s110459, s110469, s110477, s110486, s110550, s110558, s110703, s110873, s111317, s111376, s111473, s111504, s111514, s111611, s111623, s111630, s111632, s111702, s111820 and s1r1822, or an orthologue of any of the preceding.

55. A method of claim 54, wherein altering the genetic makeup of the cell comprises introducing a recombinant nucleic acid into the cell.

56. A method of claim 54, wherein the cell is a cyanobacterial cell.

57. A method of claim 54, wherein the cell is a bacterium selected from the group consisting of:
Synechocystis sp., Synechococcus sp., Ralstonia eutropha, Alcaligenes latus, Azotobacter vinelandii, Anacystis nidulans and recombinant Escherichia coli.

58. A method of claim 54, wherein the polyhydoxyalkanoate is selected from the group consisting of: polyhydroxyproprionate, polyhydroxybutyrate, polyhydroxyvalerate, polyhydroxycaproate, polyhydroxyheptanoate, polyhydroxyoctanoate, polyhydroxynonanoate, polyhydroxydecanoate, polyhydroxyundecanoate, polyhydroxydodecanoate and a mixed polymer of one or more of the forgoing polymers.

59. A bacterium comprising a recombinant nucleic acid construct comprising a coding sequence of a gene represented by an index number selected from the group consisting of: s110008, s110010, s110039, s110322, s110361, s110373, s110374, s110379, s110385, s110396, s110459, s110469, s110477, s110486, s110550, s110558, s110703, s110873, s111317, s111376, s111473, s111504, s111514, s111611, s111623, s111630, s111632, s111702, s111820 and s1r1822, or an orthologue of any of the preceding.

60. The bacterium of claim 59, wherein the bacterium is selected from the group consisting of:
Synechocystis sp., Synechococcus sp., Ralstonia eutropha, Alcaligenes latus, Azotobacter vinelandii, Anacystis nidulans and Escherichia coli.

61. A method of producing a polyhydroxyalkanoate comprising:
(a) growing a culture of cells of claim 59 under conditions suitable for the production of a polyhydroxyalkanoate; and
(b) obtaining a polyhydroxyalkanoate from the culture.

62. A method of claim 61, further comprising refining the polyhydroxyalkanoate to obtain a purer form of polyhydroxyalkanoate.

63. A method for determining whether a sample contains a hyperproliferative cell comprising:
a) determining a level of gene expression of at least one gene in a sample, wherein the at least one gene is selected from the group consisting of Neuromedin U; Aldehyde dehydrogenase 9 (Human gamma-aminobutyraldehyde dehydrogenase E3 isozyme); Fibroblast growth factor 8; Human epidermal growth factor receptor (HER3); Translocase of outer mitochondrial membrane 34; KIAA0089; Monoamine oxidase B; Zinc finger protein 273; clone 1D2; Aldehyde dehydrogenase 10 (fatty aldehyde dehydrogenase); Carboxylesterase 2 (intestine, liver); Gro2 oncogene; Diazepam binding inhibitor; Cadherin 17; TAL1 (SCL) interrupting locus; Crystallin alpha B; 5T4 oncofetal trophoblast glycoprotein; Deoxyribonuclease I-like 3; Heat-shock protein 90-kDa; Smg GDS-associated protein; Cytochrome c oxidase subunit Vb (coxVb); Wilm Tumor-Related Protein; TYRO3 protein tyrosine kinase; FAT tumor suppressor; Creatine kinase, mitochondrial 1; Transcription factor 20; MHC class I polypeptide related sequence A; KIAA0018 gene product 1; Lectin galactoside-binding, soluble, 7 (galectin 7); Tenascin-R (restrictin, janusin); CD1A antigen, a polypeptide; Beta-Hexosaminidase, Alpha Polypeptide, Abnormal Splice Mutation; clone 1A7; KIAA0172 gene; Myxovirus (influenza) resistance 2, homolog of murine; Lysophospholipase like; Interleukin-8 receptor type B, splice variant IL8RB9; keratin 4; and Runt-related transcription factor, and wherein the level of gene expression of the at least one gene allows classification of an oral keratinocyte as hyperproliferative or non-hyperproliferative with a misclassification rate of 40% or lower;
b) comparing the level of gene expression of said at least one gene to a first control level of gene expression of said at least one gene as measured in a hyperproliferative cell; and
c) comparing the level of gene expression of the at least one gene to a second control level of gene expression of said at least one gene as measured in a non-hyperproliferative cell;
wherein a sample contains a hyperproliferative cell if the level of gene expression of the at least one gene is more mathematically similar to the first control level of gene expression than to the second control level of gene expression.

64. The method of claim 63, wherein the expression levels of at least 2 genes are determined and compared in steps (a), (b), and (c).

65. The method of claim 63, wherein the misclassification rate is 15% or lower.

66. The method of claim 63, wherein the hyperproliferative cell is a cancer cell.

67. The method of claim 63, wherein the hyperproliferative cell is an oral cancer cell.

68. A method for determining whether a sample contains a hyperproliferative, cell comprising:
a) determining a level of gene expression of at least two genes in a sample, wherein said at least two genes are selected from the group consisting of Neuromedin U; Aldehyde dehydrogenase 9 (Human gamma-aminobutyraldehyde dehydrogenase E3 isozyme); Fibroblast growth factor 8; Human epidermal growth factor receptor (HER3); Translocase of outer mitochondrial membrane 34; KIAA0089; Monoamine oxidase B; Urokinase plasminogen activator; Zinc finger protein 273; clone 1D2; Aldehyde dehydrogenase 10 (fatty aldehyde dehydrogenase); Carboxylesterase 2 (intestine, liver); Gro2 oncogene; Diazepam binding inhibitor; Cadherin 17; TAL1 (SCL) interrupting locus; Crystallin alpha B; 5T4 oncofetal trophoblast glycoprotein; Deoxyribonuclease I-like 3; Heat-shock protein 90-kDa; Smg GDS-associated protein; Cytochrome c oxidase subunit Vb (coxVb); Wilm Tumor-Related Protein; TYRO3 protein tyrosine kinase; FAT tumor suppressor; Creatine kinase, mitochondrial 1; Ferritin, light polypeptide; Transcription factor 20; MHC class I polypeptide related sequence A; KIAA0018 gene product 1; Lectin galactoside-binding, soluble, 7 (galectin 7); Tenascin-R (restrictin, janusin); CD1A antigen, a polypeptide; Cytochrome P4502C9 subfamily IIC (mephytoin4-hydroxylase), polypeptide 9; Phospholipase A2, group VII; Beta-Hexosaminidase, Alpha Polypeptide, Abnormal Splice Mutation; clone 1A7; KIAA0172 gene; Interleukin 8 receptor, beta; Myxovirus (influenza) resistance 2, homolog of murine; Lysophospholipase like; Interleukin-8 receptor type B, splice variant IL8RB9; keratin 4; Runt-related transcription factor; and Cathepsin L; and wherein the level of gene expression of said at least two genes allows classification of an oral keratinocyte as hyperproliferative or non-hyperproliferative with a misclassification rate of 40% (30%, 20%, 15%, 10%) or lower;
b) comparing the level of gene expression of said at least two genes to a first control level of gene expression of said at least two genes as measured in a hyperproliferative cell; and
c) comparing the level of gene expression of the at least two genes to a second control level of gene expression of said at least two genes as measured in a non-hyperproliferative cell;
wherein a sample contains a hyperproliferative cell if the level of gene expression of the at least one gene is more mathematically similar to the first control level of gene expression than to the second control level of gene expression.

69. A method for classifying a leukemia sample comprising:
a) determining a level of gene expression of at least one gene in a sample, wherein said at least one gene is selected from the group consisting of U05259, M89957, M84371, D88270, X58529, M28170, M31523, M11722, J03473, X03934, U23852, X00437, M23323, X59871, X76223, D00749, L05148, U14603, M37271, M26692, M12886, J05243, X69398, U67171, X04145, L10373, U16954, J04132, M28826, HG4128, X87241, U50743, M13792, L47738, X95735, X17042, M23197, M84526, L09209, U46499, M27891, M16038, M63138, M55150, M22960, M62762, X61587, and U50136, and wherein the level of gene expression of said at least one gene allows classification of a leukemia as AML, B-ALL or T-ALL with a misclassification rate of 40% or lower;
b) comparing the level of gene expression of said at least one gene to a first control level of gene expression of said at least one gene as measured in an AML cell;
c) comparing the level of gene expression of said at least one gene to a second control level of gene expression of said at least one gene as measured in a B-ALL cell; and
d) comparing the level of gene expression of said at least one gene to a third level of gene expression of said at least one gene as measured in a T-ALL cell;
wherein the leukemia is classified as AML, B-ALL or T-ALL depending on whether the level of gene expression of the at least one gene is more mathematically similar to the first control level of gene expression; the second control level of gene expression; or the third control level of gene expression.

70. The method of claim 69, wherein the expression levels of at least 2 genes are determined and compared in steps (a), (b), (c) and (d).

71. The method of claim 69, wherein the misclassification rate is 15% or lower.

72. A method for classifying a leukemia sample comprising:
a) determining a level of gene expression of at least one gene in a sample, wherein said at least one gene is selected from the group consisting of M89957, M84371, D88270, X58529, M28170, M11722, J03473, X03934, U23852, X00437, M23323, X59871, X76223, D00749, LOS 148, U14603, M37271, M26692, M12886, J05243, X69398, U67171, X04145, L10373, U16954, J04132, M28826, HG4128, X87241, U50743, L09209, U46499, M22960, and X61587, and wherein the level of gene expression of said at least one gene allows classification of a leukemia as AML, or ALL with a misclassification rate of 40% or lower;
b) comparing the level of gene expression of said at least one gene to a first control level of gene expression of said at least one gene as measured in an AML cell;
c) comparing the level of gene expression of said at least one gene to a second control level of gene expression of said at least one gene as measured in an ALL cell; and
wherein the leukemia is classified as AML or ALL depending on whether the level of gene expression of the at least one gene is more mathematically similar to the first control level of gene expression or the second control level of gene expression.

73. A method for identifying a candidate therapeutic agent for the treatment of a hyperproliferative disorder comprising:
(a) contacting a hyperproliferative cell with a test therapeutic agent;
(b) determining a level of gene expression of a gene in the cell, wherein said gene is selected from the group consisting of Neuromedin U; Aldehyde dehydrogenase 9 (Human gamma-aminobutyraldehyde dehydrogenase E3 isozyme); Fibroblast growth factor 8; Human epidermal growth factor receptor (HER3); Translocase of outer mitochondrial membrane 34; KIAA0089; Monoamine oxidase B; Urokinase plasminogen activator; Zinc finger protein 273; clone 1D2; Aldehyde dehydrogenase 10 (fatty aldehyde dehydrogenase); Carboxylesterase 2 (intestine, liver); Gro2 oncogene; Diazepam binding inhibitor; Cadherin 17; TAL1 (SCL) interrupting locus; Crystallin alpha B; 5T4 oncofetal trophoblast glycoprotein; Deoxyribonuclease I-like 3; Heat-shock protein 90-kDa; Smg GDS-associated protein; Cytochrome c oxidase subunit Vb (coxVb); Wilm Tumor-Related Protein; TYRO3 protein tyrosine kinase; FAT tumor suppressor; Creatine kinase, mitochondrial 1; Ferritin, light polypeptide; Transcription factor 20; MHC class I polypeptide related sequence A; KIAA0018 gene product 1; Lectin galactoside-binding, soluble, 7 (galectin 7); Tenascin-R (restrictin, janusin); CD 1A antigen, a polypeptide; Cytochrome P4502C9 subfamily IIC (mephytoin4-hydroxylase), polypeptide 9; Phospholipase A2, group VII; Beta-Hexosaminidase, Alpha Polypeptide, Abnormal Splice Mutation; clone 1A7; KIAA0172 gene; Interleukin 8 receptor, beta; Myxovirus (influenza) resistance 2, homolog of murine; Lysophospholipase like; Interleukin-8 receptor type B, splice variant IL8RB9; keratin 4; Runt-related transcription factor; and Cathepsin L; and wherein the level of gene expression of said gene allows classification of an oral keratinocyte as hyperproliferative or non-hyperproliferative with a misclassification rate of 40% or lower; and
(c) determining whether the expression level of said gene is more mathematically similar to that of a proliferative cell or a non-hyperproliferative cell, wherein a test therapeutic agent that causes the expression level of the gene in the hyperproliferative cell to more closely resemble the expression level of the gene in a non-hyperproliferative cell is a candidate therapeutic agent.
